(19) United States
(12) Patent Application Publication
(10) Pub. No.: US 2013/0281876 A1
(43) Pub. Date: Oct. 24, 2013

(54) THERAPIES FOR IMPROVING PULMONARY FUNCTION

(71) Applicants: Raffaella Faggioni, Hayward, CA (US); Richard May, Cambridge (GB); Chris Kell, Cambridge (GB); Nester Molfino, Potomac, MD (US); Lorin Roskos, Gaithersburg, MD (US)

(72) Inventors: Raffaella Faggioni, Hayward, CA (US); Richard May, Cambridge (GB); Chris Kell, Cambridge (GB); Nester Molfino, Potomac, MD (US); Lorin Roskos, Gaithersburg, MD (US)

(73) Assignee: MEDIMMUNE LIMITED, Cambridge (GB)

(21) Appl. No.: 13/879,261

(22) PCT Filed: Oct. 14, 2011
(86) PCT No.: PCT/EP11/67947

§ 371 Date: Jul. 8, 2013

Related U.S. Application Data
(60) Provisional application No. 61/393,677, filed on Oct. 15, 2010.
(60) Provisional application No. 61/508,723, filed on Jul. 18, 2011.

(51) Int. Cl. A61B 5/087; A61K 45/06; A61K 39/395

(52) U.S. Cl. CPC A61B 5/087; A61K 39/3955; A61K 45/06
USPC 600/538; 424/142.1; 424/136.1; 424/133.1; 424/85.2; 514/44 A; 424/158.1; 424/172.1

(57) ABSTRACT

The disclosure relates to IL-13 antagonists, in particular anti-human-IL-13 antibodies and human-IL-13-binding fragments thereof, and their use for improvement, maintenance or reduction in the rate of decline of pulmonary function in a subject with impaired pulmonary function, which may be associated with uncontrolled moderate-to-asthma, COPD or IPF.

### FIELD OF THE INVENTION

The invention relates to the use of an IL-13 antagonist, in particular an anti-IL-13 antibody, for improving, maintaining or reducing the rate of decline of pulmonary function, as assessed by a spirometric method. The invention further relates to the use of an IL-13 antagonist for the treatment of asthma in a subject with detectable IL-13 in sputum. The invention further relates to a method for identification of a subject with asthma likely to benefit from treatment with an IL-13 antagonist, said subject having detectable IL-13 in sputum.

### BACKGROUND TO THE INVENTION

Respiratory disorders, such as asthma, chronic obstructive pulmonary disease (COPD) and idiopathic pulmonary fibrosis are associated with a decrease in pulmonary function; this impairment of pulmonary function can be measured by spirometry. Measurement of lung function provides an assessment of the severity of airflow limitation, its reversibility and its variability in diseases such as asthma, COPD, IPF and bronchiectasis important measures of pulmonary function these diseases are FEV₁ (forced expiratory volume in the first second), this is the volume of air that can be forced out in one second after taking a deep breath, and FVC (forced vital capacity).

Asthma is a chronic airway disorder that is characterized by variable and reversible airway obstruction, airway inflammation, excessive mucus production and airway hyper-responsiveness (AHR) that leads to episodes of wheezing, breathlessness, chest tightness and coughing. Progressive pathological airway remodelling and scarring may occur in persistent asthma resulting in partially reversible or irreversible airway obstruction.

Airways hyper-responsiveness (AHR) is the exaggerated constriction of the airways to non-specific stimuli such as cold air. Both AHR and mucus overproduction are thought to be responsible for the variable airway obstruction that leads to the shortness of breath characteristic of asthma attacks (exacerbations) and which is responsible for the mortality associated with this disease (around 2000 deaths/year in the United Kingdom).

Current British Thoracic Society (BTS) and Global Initiative for Asthma (GINA) guidelines suggest a stepwise approach to the treatment of asthma. Most patients with asthma have mild-to-moderate disease that is controlled at present by “standard of care” therapies: inhaled corticosteroids combined with β₂-adrenoceptor agonists or leukotriene inhibitors. However, patients may not comply with the treatment regime which reduces the effectiveness of treatment. In more severe disease, long-term treatment with oral corticosteroids may be required, but is associated with side effects such as osteoporosis, diabetes and oral candidiasis.

Although conventional therapies alleviate mild-to-moderate disease in many patients, in a subset of asthmatic patients (approximately 25% of the moderate-to-severe population), asthma continues to be poorly controlled in terms of ongoing symptoms, frequent exacerbations, persistent and variable airway obstruction and frequent requirement for β₂-agonists, despite adequate treatment. This results in considerable impact on quality of life, disproportionate use of healthcare resources, and adverse effects from regular systemic steroid use. Hospitalization of these patients, who represent around 6% of the UK asthma population, as a result of severe exacerbations accounts for the majority of the significant economic burden of asthma on healthcare authorities. Thus, there is a clear need for new treatments for asthma, particularly in subjects with more severe asthmatic disease, who often gain very limited benefit from either higher doses of inhaled or oral corticosteroids recommended by asthma guidelines. Additionally, there is a need for new treatments for children with asthma, as treatment with high dose corticosteroids is associated with reduced growth rates.

It is believed that the pathology of asthma is caused by ongoing Th2 lymphocyte mediated inflammation that results from inappropriate responses of the immune system to harmless antigens. Interleukin-13 (IL-13) has emerged as a key pleiotropic Th2 cytokine that contributes to the development and maintenance of all aspects of the asthma phenotype. Human Interleukin (IL)-13 is a 114 amino acid protein with an unmodified molecular mass of approximately 12 kDa. IL-13 is most closely related to IL-4 with which it shares 30% sequence similarity at the amino acid level. IL-13 shares receptor components and many biological properties with interleukin 4 (IL-4). IL-13 receptors (heterodimers of IL-13Rα1 and IL-4Rα) are expressed on the leukocyte subsets (eosinophils, neutrophils, B cells, NKT cells) and structural cells (epithelial cells, fibroblasts, smooth muscle) considered relevant in asthma. The action of IL-13 on these cells is to stimulate processes that promote airway hyperresponsiveness, inflammation, and underlying airway structural changes including fibrosis and mucus hypersecretion that are cardinal features of asthma (Hershey, 2003).

IL-13 mRNA or protein levels are elevated in bronchial biopsies, sputum, and BAL fluid from asthmatics compared to control subjects (Humbert et al., 1997; Kotsimbos et al., 1996; Komai-Koma et al., 2001; Naseer et al., 1997) and further increased in BAL samples from allergen challenged asthmatics (Huang et al., 1995; Kroegel et al., 1996). IL-13 protein expression in the lung is also correlated with both asthma severity and control. Mild steroid-naïve asthmatics have upregulated IL-13 in the sputum compared to “normals”, as do severe asthmatics who have poor control despite standard of care up to, and including, oral corticosteroids; moderate asthmatics (well controlled by inhaled corticosteroids) are not reported to have upregulated IL-13 expression (Saha et al., 2008).

Genetic polymorphisms in the IL-13 pathway are also associated with both atopy and asthma; IL-13 Q130R has been associated with asthma, atopy, and raised serum IgE (Heinzmann et al., 2000; Liu et al., 2000; Kauppi et al., 2001) activating the signaling IL-13 receptor (Rα1) and downstream functions more efficiently, whilst binding to the decoy receptor (Rα2) less efficiently, than wild-type IL-13 (Vladich et al., 2005). Similar polymorphisms associated with risk of atopy and asthma have been observed in the IL-13 promoter (Howard et al., 2001 and van der Pouw Kraan et al., 1999), IL4Rα (Howard et al., 2002), and in the key signalling molecule downstream of the IL-13 receptor—STAT6 (Tamura et al., 2003). The IL-13 variant (Q130R) and antibodies to this variant are discussed in WO 01/62933.

Animal model data from models mimicking aspects of human asthma support the hypothesis that IL 13 is a key mediator in the development and maintenance of the asthma phenotype. Administration of recombinant IL 13 to the airway of allergen-naïve mice results in AHR, airway inflammation, and mucus production (Wills-Karp et al., 1998; Grunig et al., 1998; Venkayya et al., 2002). Similar pathologies are seen in transgenic mice in which IL-13 is selectively overexpressed in the lung although longer-term exposure to IL-13 also results in fibrosis (Zhu et al., 1999). The same ‘asthmatic’ phenotype occurs following allergen challenge in sensitised mice and is also associated with IL-13. Soluble IL-13Rα2, a potent IL-13 neutraliser, has been shown to inhibit AHR, mucus hypersecretion, and pulmonary inflammation (Wills-Karp et al., 1998; Grunig et al., 1998). NHP segmental antigen challenge studies have also shown an anti-inflammatory effect for an IL-13 neutralising antibody (Bree et al., 2007).

Given the range of cells involved in asthma on which IL-13 is known to act, and the pathogenic functions ascribed to this interleukin, a significant role for IL-13 in asthma can be expected. Coupled with the evidence of a relationship between IL-13 expression and disease severity in patients (Saha et al., 2008), neutralisation of IL-13 is a credible approach to the treatment of asthma.

Chronic Obstructive Pulmonary Disease (COPD) includes patient populations with varying degrees of chronic bronchitis, small airway disease and emphysema and is characterized by progressive irreversible lung function decline that responds poorly to current asthma based therapies. The incidence of COPD has risen dramatically in recent years to become the fourth leading cause of death worldwide (World Health Organization). COPD therefore represents a large unmet medical need. The underlying causes of COPD remain poorly understood. It has been proposed that there is a common susceptibility to COPD and asthma and therefore, that similar mechanisms may contribute to the pathogenesis of both disorders.

Over-expression of IL-13 in the mouse lung has been demonstrated to cause emphysema, elevated mucus production and inflammation, reflecting aspects of human COPD. Furthermore, AHR, an IL-13 dependent response in murine models of allergic inflammation, has been shown to be predictive of lung function decline in smokers. A link has also been established between an IL-13 promoter polymorphism and susceptibility to develop COPD.

The signs are, therefore, that IL-13 plays an important role in the pathogenesis of COPD, particularly in patients with asthma-like features, including AHR and eosinophilia. The mRNA levels of IL-13 have been shown to be higher in autopsy tissue samples from subjects with a history of COPD when compared to lung samples from subjects with no reported lung disease. In another study, raised levels of IL-13 were demonstrated by immunohistochemistry in peripheral lung sections from COPD patients.

IL-13 has been associated with other fibrotic conditions. Increased levels of IL-13 have been measured in the serum of patients with systemic sclerosis and in BAL samples from patients affected with other forms of pulmonary fibrosis. Correspondingly, over-expression of IL-13 in the mouse lung has been reported to result in pronounced fibrosis. The contribution of IL-13 to fibrosis in tissues other than the lung has been extensively studied in a mouse model of parasite-induced liver fibrosis.

Tralokinumab (an IgG4 monoclonal antibody) is a highly potent and specific IL-13-neutralising antibody, with a 165 μM affinity for human IL-13, that neutralises human and cynomolgus IL-13 with near equivalent potency, but does not neutralise mouse IL-13, nor the most homologous cytokine to IL-13, IL-4. CAT-354 also neutralises the variant IL-13, Q130R, which has been associated with atopy and asthma as well as endogenous (mammalian glycosylated) human IL-13. Tralokinumab (BAK502G9) is described in International Patent Application Publication No. WO 2005/007699, incorporated herein by reference.

It is an object of the invention to provide IL-13 antagonists, in particular anti-IL-13 antibodies such as tralokinumab, or IL-13 neutralizing fragments thereof, for improvement, maintenance or reduction in decline of pulmonary function, in particular as assessed by FEV₁, or FVC, in subjects with impaired pulmonary function, which may be due to asthma, COPD or pulmonary fibrosis. It is a further object of the invention to provide an IL-13 antagonist for the treatment of asthma in a subject, wherein said subject has upregulated airway IL-13, for example, based on detectable IL-13 in sputum. It is a further object of the invention to provide a method for identification of a subject with asthma likely to benefit from treatment with an IL-13 antagonist, wherein said subject has upregulated airway IL-13, for example, based on detectable IL-13 in sputum.

### STATEMENT OF INVENTION

The invention provides an IL-13 antagonist for improvement, maintenance or reduction in the rate of decline of pulmonary function in a subject with impaired pulmonary function.

The invention provides a method of treating a subject with impaired pulmonary function, said method comprising administering to said subject an IL-13 antagonist in an amount sufficient to improve, maintain or reduce the rate of decline of pulmonary function.

The invention provides for the use of an IL-13 antagonist in the manufacture of a medicament for the treatment of impaired pulmonary function to improve, maintain or reduce the rate of decline of pulmonary function in a subject in need thereof.

The invention provides a method of improving, maintaining or reducing the rate of decline in pulmonary function in a subject with impaired pulmonary function, said method comprising administering to said subject an IL-13 antagonist.

Additionally the invention provides a composition for the treatment of impaired pulmonary function to improve, maintain or reduce the rate of decline of pulmonary function in a subject with impaired pulmonary function, said composition comprising an IL-13 antagonist and a pharmaceutically acceptable excipient.

The invention provides an IL-13 antagonist for the treatment of asthma in a subject, wherein said subject has upregulated airway IL-13, for example, based on detectable IL-13 in sputum.

The invention provides a method for treating asthma in a subject, wherein said subject has detectable IL-13 in sputum, said method comprising administering to said subject an IL-13 antagonist in an amount sufficient to ameliorate asthma symptoms.

The invention provides for the use of an IL-13 antagonist in the manufacture of a medicament for the treatment of asthma in a subject, wherein said subject has detectable IL-13 in sputum.

The invention provides a method for the identification of a subject with asthma likely to benefit from treatment with an IL-13 antagonist, said method comprising testing for the presence of IL-13 in a sample of sputum from said subject, wherein a subject with detectable IL-13 in sputum is likely to benefit from treatment with an IL-13 antagonist.

A beneficial response to treatment of asthma may be assessed by consideration of asthma symptoms, e.g. by an ACQ method, such as ACQ-6, and/or by spirometric assessments as described herein. Subjects that benefit from treatment with an IL-13 antagonist experience an amelioration of their asthma symptoms. As described herein, subjects with asthma who had detectable IL-13 in their sputum experienced an amelioration of asthma symptoms following treatment with an IL-13 antagonist. Methods of treatment and uses described herein may comprise a step, performed prior to treatment of a subject with an IL-13 antagonist, whereby a sample of sputum from the subject is tested for the presence of IL-13. The inventors have found that presence of IL-13 in sputum defines a sub-set of asthmatics in which IL-13 antagonist therapy is particularly beneficial, as described herein.

The invention provides a composition for the treatment of asthma in a subject, wherein said subject has detectable IL-13 in sputum, said composition comprising an IL-13 antagonist and a pharmaceutically acceptable excipient.

Thus pharmaceutical compositions according to the present invention, and for use in accordance with the present invention, may comprise, in addition to active ingredient, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. intravenous. For injection, the active ingredient will be in the form of a parenterally acceptable aqueous solution which is pyrogen-free and has suitable pH, isotonicity and stability. Preservatives, stabilisers, buffers, antioxidants and/or other additives may be included, as required.

Compositions of the present invention may be formulated in liquid or solid forms. Solid forms may be provided for reconstitution prior to administration by intravenous or subcutaneous injection, or for inhalation. The composition employed will depend on the physicochemical properties of the molecule and the route of delivery. Formulations may include excipients, or combinations of excipients, for example: sugars, amino acids and surfactants. Liquid formulations may include a wide range of antibody concentrations and pH. Solid formulations may be produced by lyophilisation, spray drying, or drying by supercritical fluid technology, for example. Formulations will depend upon the intended route of delivery: for example, formulations for pulmonary delivery may consist of particles with physical properties that ensure penetration into the deep lung upon inhalation.

In a particularly preferred embodiment, tralokinumab is formulated at 150 mg/mL in 50 mM sodium acetate, 85 mM sodium chloride, 0.01% (w/v) plant-derived polysorbate 80 at pH 5.5.

A composition in accordance with the invention may be administered alone or in combination with other treatments, either simultaneously or sequentially dependent upon the condition to be treated.

Treatment may be given by injection, for example, subcutaneously, or intravenously, or by inhalation. It is envisaged that treatment will not be restricted to use in the clinic. Therefore, subcutaneous injection using an administration device is envisaged.

Accordingly, the invention also provides an administration device for the treatment of impaired pulmonary function to improve, maintain or reduce the rate of decline of pulmonary function in a subject in need thereof, said device comprising an IL-13 antagonist. An administration device may comprise a composition for the treatment of impaired pulmonary function to improve, maintain or reduce the rate of decline of pulmonary function in a subject in need thereof, said composition comprising an IL-13 antagonist and a pharmaceutically acceptable excipient.

The invention also provides an administration device for the treatment of asthma in a subject, wherein said subject has detectable IL-13 in sputum, said device comprising an IL-13 antagonist. An administration device may comprise a composition for the treatment of asthma in a subject, wherein said subject has detectable IL-13 in sputum, said composition comprising an IL-13 antagonist and a pharmaceutically acceptable excipient.

In some embodiments of the invention, a detectable level of IL-13 in sputum may be the presence of ≧1 pg/mL IL-13. The presence of IL-13 in sputum can be detected using an ELISA method. The limit of quantification using ELISA methods is about 1 pg/mL, hence subjects with a concentration of IL-13 in sputum that is <1 pg/may be deemed to not have detectable IL-13 in sputum. Methods suitable for detection of IL-13 in sputum include: western blot, proteomic techniques such as mass spectrometry, MALDI-TOF and 2D-DIGE; antibody- or receptor-based protein identification techniques, which include ELISAs in the format of Luminex® assays (Luminex Corp., Austin, Tex., USA) based on xMAP technology (multi-analyte profiling beads) enabling the detection and quantitation of multiple RNA or protein targets simultaneously; the xMAP system combines a flow cytometer, fluorescent-dyed microspheres (beads), lasers and digital signal processing to efficiently allow multiplexing of up to 100 unique assays within a single sample and in the format of MSD® cytokine assays (Meso Scale Discovery, Gaithersburg, Md., USA) that employ electrochemiluminescent detection; sputum-driven cell-based assays to identify IL-13 in sputum, such as TF1 proliferation blocked with an IL-13 neutralising reagent or STAT-6 phosphorylation and measurement of downstream mediators, e.g. eotaxin, TARC, MCP4. The amount corresponding to a detectable level of IL-13 in sputum may vary due to the known limit of quantitation of a method known in the art such as any of those described above, or may be further refined based on improvement in the limits of quantitation of methods known in the art such as those described above, or other methods suitable to detect levels of a molecule such as IL-13.

An administration device in accordance with the invention may be selected from a pre-filled syringe and an auto-injector device for sub-cutaneous administration, which may be designed to permit “home” administration.

In respiratory diseases such as asthma, COPD, and IPF, pulmonary function tests (PFT) are used to assess lung function and quantify the amount of damage to the lungs; PFT can also be used to assess the progression of lung disease and the effectiveness of treatment. Spirometry testing is the most commonly used of all pulmonary function tests. It is a convenient non-invasive procedure, generally performed with a hand-held spirometer. It is normally the clinician's first choice when attempting to diagnose a respiratory problem. Spirometry requires the patient, after all air has been expelled, to inhale deeply; this is then followed by a rapid exhalation, so that all the air is expelled from the lungs. Results of spirometry tests vary, but are based on predicted values of a standardized, healthy population. Common Terminology in spirometry testing is as follows:

VC-Vital Capacity, this is the amount of air that can be forcibly exhaled from the lungs after a full inhalation.
FVC-Forced Vital Capacity, this is the amount of air that can be forcibly exhaled from the lungs after taking the deepest breath possible.
FEV₁-Forced Expiratory Volume in One Second, this is the amount of air which can be forcibly exhaled from the lungs in the first second of a forced exhalation.
FEV₁/FVC, the ratio of FEV₁ to FVC expressed as a fraction (previously this was expressed as a percentage), this indicates what fraction of the total amount of air is exhaled from the lungs during the first second of forced exhalation.
PEF Peak Expiratory Flow, this is used as a measure to determine if treatment is effective in improving airway diseases, in particular in asthma and COPD.
FEF Forced Expiratory Flow, this is a measure of how much air can be exhaled from the lungs; it is an indicator of large airway obstruction.
MVV Maximal Voluntary Ventilation, a value for this is determined by having the patient inhale and exhale as rapidly and fully as possible in 12 seconds. The results reflect the status of the muscles used for breathing, how stiff the lungs are and if there is any resistance in the airways when breathing. This test tells indicates how strong a patient's lungs are prior to surgery. If patients demonstrate poor performance on this test, it suggests that respiratory complications may occur after surgery.

In asthma, measurement of lung function using spirometry, in particular measurement of FEV₁ and/or Peak expiratory flow is used in both diagnosis and monitoring to confirm airflow limitation, and to demonstrate reversibility of lung function abnormalities following administration of a bronchodilator. The degree of reversibility in forced expiratory volume in one second (FEV₁) indicative of a diagnosis of asthma is usually accepted as ≧12% and ≧200 mL from the pre-bronchodilator value. As many lung diseases may result in reduced FEV₁, a useful assessment of airflow limitation in asthma is the ratio of FEV₁ to forced vital capacity (FVC). The FEV₁/FVC ratio is normally >0.75-0.80, and possibly >0.90 in children. Lower values suggest airflow limitation. PEF measurements are made using a peak flow meter are also a useful aid to diagnosis and monitoring of asthma. The terms “reversibility” and “variability” refer to changes in symptoms accompanied by changes in airflow limitation that occur spontaneously or in response to treatment. The term reversibility is generally applied to rapid improvements in FEV₁ (or PEF) measured within minutes after inhalation of a rapid-acting bronchodilator, e.g. after 200-400 μg salbutamol (albuterol), or more sustained improvement over days or weeks after the introduction of effective controller treatment, such as inhaled glucocorticosteroids. Variability refers to improvement or deterioration in symptoms and lung function occurring over time. Variability may be experienced over the course of 1 day (when it is called diurnal variability), from day to day, from month to month, or seasonally.

In accordance with the invention, it is particularly preferred that pulmonary function is assessed by spirometry, e.g. by measuring FEV₁ (asthma) and/or FVC (COPD/IPF), using methods standard in the art.

FEV₁ can be measured using the Masterscope® CT Spirometer using a protocol following the ATS/ERS recommendations 2005 (Eur Respir J; 26: 319-338). FEV₁ is the maximal volume of air exhaled in the first second of a forced expiration from a position of full inspiration, expressed in litres at BTPS (Body temperature (i.e. 37° C.), ambient pressure, saturated with water vapour). FVC is the maximal volume of air exhaled with maximally forced effort from a maximal inspiration, i.e. vital capacity performed with a maximally forced expiratory effort, expressed in litres at body temperature and ambient pressure saturated with water vapour.

In asthma, an IL-13 antagonist in accordance with the invention may be employed to treat impaired pulmonary function to improve pulmonary function. When assessed by FEV₁, the improvement can be expressed as mean percent change in FEV₁ from baseline, preferably in the range of from 5% to 18%; for example, 5, 6, 7, 8, 9, 10, 11, 12, 13, 14, 15, 16, 17, 18%; such as at least 5, 6, 7, 8, 9, 10, 11, 12, 13, 14, 15, 16, 17, 18%.

In COPD or IPF, an IL-13 antagonist in accordance with the invention may be employed to treat impaired pulmonary function to maintain or slow the rate of decline in pulmonary function.

In certain embodiments of the invention amelioration of asthma in a subject is assessed by a composite measure of one or more asthma symptoms selected from the group consisting of: night-time waking, symptoms on waking, activity limitation, shortness of breath, wheezing, and short-acting β₂-agonist use. Amelioration of asthma in a subject can be assessed by asthma control questionnaire ACQ-6 (see Juniper E F, O'Byrne P M, Guyatt G H, Ferrie P J, King D R. Development and validation of a questionnaire to measure asthma control. Eur Respir J 1999; 14: 902-7, the entire contents of which are incorporated herein by reference). Amelioration of asthma in a subject can be assessed by reduction in ACQ-6 score compared to baseline ACQ-6 score before administration of the IL-13 antagonist. A reduction in mean ACQ-6 score of 0.5 relative to baseline score is considered to be indicative of a clinically meaningful of amelioration of asthma, whereas a score of 1.5 indicates uncontrolled asthma.

In preferred embodiments of the invention, the IL-13 antagonist is an anti-human-IL-13 antibody or a human-IL-13-binding fragment thereof. Preferably, the anti-IL-13 antagonist is an anti-human-IL-13 antibody or a human-IL-13-binding fragment thereof, selected from:

(a) tralokinumab (BAK502G9, CAT-354) antibody;
(b) a human-IL-13 binding fragment of tralokinumab comprising an antibody antigen-binding site which is composed of a human antibody VH domain (SEQ ID NO: 15) and a human antibody VL domain (SEQ ID NO:16) and which comprises a set of CDR's HCDR1, HCDR2, HCDR3, LCDR1, LCDR2 and LCDR3, wherein the VH domain comprises HCDR1, HCDR2 and HCDR3 and the VL domain comprises LCDR1, LCDR2 and LCDR3, wherein the HCDR1 has the amino acid sequence of SEQ ID NO: 7, the HCDR2 has the amino acid sequence of SEQ ID NO: 8, the HCDR3 has the amino acid sequence of SEQ ID NO: 9, the LCDR1 has the amino acid sequence of SEQ ID NO: 10, the LCDR2 has the amino acid sequence of SEQ ID NO: 11, and the LCDR3 has the amino acid sequence of SEQ ID NO: 12;
(c) a human-IL-13 binding fragment of tralokinumab comprising an antibody antigen-binding site which is composed of a human antibody VH domain and a human antibody VL domain which comprises a set of CDR's HCDR1, HCDR2, HCDR3, LCDR1, LCDR2 and LCDR3, wherein the VH domain comprises HCDR 1, HCDR2 and HCDR3 and the VL domain comprises LCDR1, LCDR2 and LCDR3, wherein the HCDR1 has the amino acid sequence of SEQ ID NO: 7, the HCDR2 has the amino acid sequence of SEQ ID NO: 8, the HCDR3 has the amino acid sequence of SEQ ID NO: 9, the LCDR1 has the amino acid sequence of SEQ ID NO: 10, the LCDR2 has the amino acid sequence of SEQ ID NO: 11, and the LCDR3 has the amino acid sequence of SEQ ID NO: 12;
(d) an anti-IL-13 antibody, or a human-IL-13 binding fragment thereof comprising an antibody antigen-binding site which is composed of a human antibody VH domain (SEQ ID NO: 15) and/or a human antibody VL domain (SEQ ID NO:16);
(e) an antibody or an IL-13 binding fragment thereof that competes with tralokinumab for binding to human IL-13;
(f) an antibody or an IL-13 binding fragment thereof capable of binding an epitope within the human IL-13 amino acid sequence from aspartic acid at position 99 to C-terminal asparagine at position 132 (FSSLHVRDTKIEVAQFVKDLLLHLKKLFREGRFN) of human IL-13 protein, or capable of binding an epitope within the human IL-13 sequence from aspartic acid at position 106 to C-terminal asparagine at position 132 (DTKIEVAQFVKDLLLHLKKLFREGRFN) of human IL-13 protein; and
(g) an antibody or an IL-13 binding fragment thereof that has one, two, three, four, five, or six CDRs (SEQ ID NOs: 7, 8, 9, 10, 11, 12) in common with tralokinumab.

In a preferred aspect of the anti-human-IL-13 antibody or a human-IL-13 binding fragment thereof described above, the whole antibody is an IgG4.

In accordance with the invention, an anti-human-IL-13 antibody or a human-IL-13 binding fragment thereof may comprise one or more CDRs, or a set of all 6 CDRs (HCDR1, HCDR2, HCDR3, LCDR1, LCDR2, LCDR3), selected from the CDRs of:

### BAK278D6 (SEQ ID NOS: 1, 2, 3, 4, 5, 6),

### BAK1183H4 (SEQ ID NOS: 97, 98, 99, 100, 101, 102),

### BAK1167F02 (SEQ ID NOS: 64, 65, 66, 67, 68, 69),

### BAK1111D10 (SEQ ID NOS: 91, 92, 93, 94, 95, 96),

### BAK1166G02 (SEQ ID NOS: 67, 68, 69, 70, 71, 72),

### BAK1167F04 (SEQ ID NOS: 85, 86, 87, 88, 89, 90),

### BAK1184C8 (SEQ ID NOS: 73, 74, 75, 76, 77, 78),

### BAK1185E1 (SEQ ID NOS: 79, 80, 81, 82, 83, 84), and

### BAK1185F8 (SEQ ID NOS: 103, 104, 105, 106, 107, 108).

A method, use, composition or device of the invention may comprise an anti-human-IL-13 antibody or a human-IL-13 binding fragment thereof comprising a VH domain comprising a set of CDRs and/or a VL domain comprising a set of CDRs selected from the set of HCDRs and/or set of LCDRs of BAK278D6, BAK1183H4, BAK1167F02, BAK1111D10, BAK1166G02, BAK1167F04, BAK1184C8, BAK1185E1, and BAK1185F8.

An anti-human-IL-13 antibody or a human-IL-13 binding fragment thereof, may comprise a VH and VL of a clone selected from:

### BAK278D6 (VH SEQ ID NO: 13, VL SEQ ID NO: 14),

### BAK1183H4 (VH SEQ ID NO: 37, VL SEQ ID NO: 38),

### BAK1167F02 (VH SEQ ID NO: 35, VL SEQ ID NO: 36),

### BAK1111D10 (VH SEQ ID NO: 41, VL SEQ ID NO: 42),

### BAK1166G02 (VH SEQ ID NO: 53, VL SEQ ID NO: 54),

### BAK1167F04 (VH SEQ ID NO: 43, VL SEQ ID NO: 44),

### BAK1184C8 (VH SEQ ID NO: 45, VL SEQ ID NO: 46),

### BAK1185E1 (VH SEQ ID NO: 47, VL SEQ ID NO: 48), and

### BAK1185F8 (VH SEQ ID NO: 49, VL SEQ ID NO: 50).

An anti-human-IL-13 antibody or a human-IL-13 binding fragment thereof may have HCDR1, HCDR2 and HCDR3 of the VH domain within a germ-line framework and/or LCDR1, LCDR2 and LCDR3 of the VL domain within a germ-line framework. The VH germ-line framework can be VH1 DP14. The VL germ-line framework can be VL γ3 3H.

An anti-human-IL-13 antibody or a human-IL-13 binding fragment thereof may bind a human IL-13 variant in which arginine at position 130 is replaced by glutamine. An anti-human-IL-13 antibody or a human-IL-13 binding fragment thereof may bind a non-human primate IL-13, such as rhesus or cynomolgus IL-13.

Other IL-13 antagonists that may be employed in accordance with the invention include:

(a) Anti-human-IL-13 antibodies such as Lebrikizumab (MILR1444A/RG3637, Roche/Genentech), ABT-308 (Abbott), GSK679586 (GlaxoSmithkline); QAX576 (Novartis),
(b) Anti-human-IL-13Rα1 antibodies such as Merck MK6105;
(c) IL-13—toxin conjugates, such as IL-13 PE38QQR;
(d) IL-4 muteins, such as Aerovant™ (Aerovance) (modified IL-4);
(e) anti-IL-4Rα antibodies, such as Regeneron-668 (Regeneron);
(f) nucleic acids such as AIR645 (a double-stranded oligonucleotide directed against IL-4Rα); and,
(g) IL-4/IL-13 bispecific antibodies, such as Domantis' 09/10 and DOM-1000P.

In some aspects of the invention, it is preferred that the anti-IL-13 antagonist is not also an antagonist of the interaction between IL-4 and IL4Rα, preferably the IL-13 antagonist is not an anti-IL-4Rα antibody, such as AMG317 (Amgen).

Preferably the anti-IL-13 antagonist is an anti-human-IL-13 antibody or a human-IL-13-binding fragment thereof.

Preferably, an antibody or a binding fragment thereof employed in the invention is selected from the group consisting of; an immunoglobulin molecule, a monoclonal antibody, a chimeric antibody, a CDR-grafted antibody, a humanized antibody, a Fab, a Fab', a F(ab′)2, a Fv, a disulfide linked Fv, a scFv, a single domain antibody, a diabody, a multispecific antibody, a dual-specific antibody, and a bispecific antibody.

The subject to be treated in accordance with the invention may have impaired pulmonary function associated with asthma, COPD, or idiopathic pulmonary fibrosis (IPF). Asthma may be classified as atopic asthma non-atopic asthma, moderate to severe asthma, moderate to severe asthma uncontrolled by inhaled or oral corticosteroids and asthma at GINA score 5, 4, 3, 2, or 1. Subjects with asthma and may be undergoing concomitant therapy with one or more further therapeutic selected from an inhaled corticosteroid, long-acting beta2 antagonist (LABA), theophylline, a leukotriene antagonist, and an oral corticosteroid.

Impaired pulmonary function may be associated with COPD, such as moderate to severe COPD defined by GOLD classification II, II and IV. The spirometric criterion required for a diagnosis of COPD is an FEV₁/FVC ratio below 0.7 after Bronchodilator). The GOLD spirometric criteria for COPD severity is as follows

| | | | |
|---|---|---|---|
| I: Mild COPD | FEV1/FVC < 0.7 | FEV1 ≧ 80% | At this stage, the |
| | | predicted | patient may not be |
| | | | aware that their lung |
| | | | function is abnormal. |
| II: Moderate | FEV1/FVC < 0.7 | 50% ≦ FEV1 < | Symptoms usually |
| COPD | | 80% predicted | progress at this stage, |
| | | | with shortness of |
| | | | breath typically |
| | | | developing on |
| | | | exertion. |
| III: Severe | FEV1/FVC < 0.7 | 30% ≦ FEV1 < | Shortness of breath |
| COPD | | 50% predicted | typically worsens at |
| | | | this stage and often |
| | | | limits patients' daily |
| | | | activities. |
| | | | Exacerbations are |
| | | | especially seen |
| | | | beginning at this |
| | | | stage. |
| IV: Very | FEV1/FVC < 0.7 | FEV1 < 30% | At this stage, quality |
| Severe COPD | | predicted or | of life is very |
| | | FEV1 < 50% | appreciably |
| | | predicted plus | impaired and |
| | | chronic | exacerbations |
| | | respiratory | may be life- |
| | | failure | threatening. |

Subjects with COPD may be undergoing concomitant therapy with one or more further therapeutic selected from an inhaled corticosteroid, LABA, theophylline, an oral corticosteroid, tiotropium and roflumilast.

Impaired pulmonary function may be associated with idiopathic pulmonary fibrosis; subjects with IPF may be undergoing concomitant therapy with one or more further therapeutic selected from an inhaled corticosteroid, LABA, theophylline, a leukotriene antagonist, an oral corticosteroid and pirfenidone.

Accordingly, an IL-13 antagonist or composition comprising an IL-13 antagonist as described herein may be administered alone or in combination with other treatments, either simultaneously or sequentially dependent upon the condition to be treated.

In some aspect of the invention the subject with impaired pulmonary function is steroid naïve.

In preferred embodiments, the IL-13 antagonist, which is an anti-human-IL-13 antibody or a human-IL-13-binding fragment thereof, is administered subcutaneously at a dose in the range of from 100 to 800 mg, preferably at a dose in the range of from 100 to 600 mg; or is administered intravenously at a dose in the range of from 100 to 1800 mg, preferably at a dose in the range of from 200 to 1600 mg, most preferably at about 1500 mg. Preferably the anti-human IL-13 antibody is tralokinumab or a variant thereof or a human-IL-13 binding fragment thereof, as described herein. Suitable unit doses for sub-cutaneous administration may be selected from 150, 300, and 600 mg, preferably administered at 2 or 4 week intervals. Suitable unit doses for intravenous administration may be selected from 1000, 1200, 1400, 1500, 1600 1700 and 1800 mg, preferably administered at 2 or 4 week intervals, most preferably administered at 4 week intervals. Preferably, the IL-13 antagonist is an anti-human-IL-13 antibody or human-IL-13-binding fragment thereof, more preferably the anti-human IL-13 antibody is tralokinumab or a variant thereof or a human-IL-13 binding fragment thereof, as described herein, administered at a mg/kg dose in the range of from 1 mg/Kg to 30 mg/Kg.

The subject may be an adult, or a young adult (12-18 years' old) however the invention may also be employed to improve, maintain or reduce the rate of decline of pulmonary function in paediatric subjects, aged less than 12 years' old.

An IL-13 antagonist, preferably an anti-human-IL-13 antibody or human-IL-13-binding fragment thereof, more preferably the anti-human IL-13 antibody is tralokinumab or a variant thereof or a human-IL-13 binding fragment thereof, as described herein, may be administered in accordance with the invention at a frequency selected from every 1, 2, 3, 4, 6, 8, 10 and 12 weeks, preferred dosing intervals are every two or every four weeks.

Generally, an improvement in pulmonary function in asthma subjects can be detected by 15 days following initiation of treatment in accordance with the invention.

Surprisingly, the inventors found an improvement in pulmonary function as assessed by spirometry by measuring FEV₁ in moderate to severe asthma patients (GINA 4 or 5) to whom tralokinumab had been administered by subcutaneous administration, at doses of 150 mg, 300 mg or 600 mg, at dosing intervals of 2 weeks. It is believed that hitherto anti-human IL-13 antibodies have not been reported to improve pulmonary function in asthmatic patients. An early onset of improvement in pulmonary function, as assessed by FEV₁ was seen in the asthmatic subjects treated with tralokinumab, with an improvement in FEV₁ score being detectable by 15 days after the first administration of tralokinumab. Furthermore, it was found that the improvement in FEV₁ score correlated with the dose of the tralokinumab that was administered. The average improvement in FEV₁ score over baseline measurement taken before initiation of treatment was 8% in the 150 mg treatment group, 13.3% in the 300 mg treatment group and 15.2% in the 600 mg treatment group. The mean improvement in FEV₁ for all tralokinumab treatment groups combined was 12.2%.

Accordingly, the inventors have found that an improvement in pulmonary function, as assessed by spirometry, e.g., by measuring FEV₁, can be used as an indicator of a subjects' response to treatment of asthma using an anti-IL-13 antagonist, preferably using an anti-human-IL-13 antibody or human-IL-13-binding fragment thereof, e.g. tralokinumab or a variant thereof or a human-IL-13 binding fragment thereof as described herein.

Thus, the invention further provides a method for detecting a subject's response to an IL-13 antagonist treatment in asthma, comprising:

(a) assessing pulmonary function by a spirometric method prior to treatment,
(b) assessing pulmonary function by a spirometric method after treatment,
(c) wherein an improvement in pulmonary function is indicative of a beneficial response to treatment.

It is preferred that pulmonary function is assessed by measuring FEV₁ and/or PEF. Preferably, assessment of pulmonary function after treatment is performed at least 15 days after initiation of treatment.

In preferred aspects of the invention the IL-13 antagonist is an anti-human-IL-13 antibody, more preferably the anti-human IL-13 antibody is tralukinumab or a variant thereof or a human-IL-13 binding fragment thereof, as described herein, preferably administered by subcutaneous administration, preferred doses being 150 mg, 300 mg or 600 mg, with the preferred dosing interval being 2 or 4 weeks. Preferably, the improvement in pulmonary function following treatment compared to baseline is in the range of from 8% to 16%, measured at least 15 days after initiation of treatment and/or up to 12 weeks after cessation of treatment.

Preferably, the improvement in pulmonary function from baseline, as assessed by FEV₁ measurement, following subcutaneous administration at two week intervals of 150 mg tralokinumab or a variant thereof or a human-IL-13 binding fragment thereof, as described herein, is in the range of from about 6 to about 10%, e.g., about 8%. Preferably, assessment of pulmonary function after treatment is performed at least 15 days after initiation of treatment and/or up to 12 weeks after cessation of treatment.

Preferably, the improvement in pulmonary function from baseline, as assessed by FEV₁ measurement, following subcutaneous administration at two week intervals of 300 mg tralokinumab or a variant thereof or a human-IL-13 binding fragment thereof, as described herein, is in the range of from about 12 to about 14%, e.g., about 13.3%. Preferably, assessment of pulmonary function after treatment is performed at least 15 days after initiation of treatment and/or up to 12 weeks after cessation of treatment.

Preferably, the improvement in pulmonary function from baseline, as assessed by FEV₁ measurement, following subcutaneous administration at two week intervals of 600 mg tralokinumab or a variant thereof or a human-IL-13 binding fragment thereof, as described herein, is in the range of from about 14 to about 16%, e.g., about 15.2%. Preferably, assessment of pulmonary function after treatment is performed at least 15 days after initiation of treatment and/or up to 12 weeks after cessation of treatment.

All subjects were followed for 12 weeks after the last dose of tralokinumab and underwent spirometry testing at days 127 and day 169. Surprisingly, the increases in FEV₁ that were observed in tralokinumab-treated subjects at day 92 were maintained at both these post-dosing time points. The mean change from baseline in FEV₁ at day 169 was 0.101 L in the placebo group and 0.218 L in the combined subcutaneous tralokinumab treatment group (p=0.096). The numerical increases in FVC and PEF observed at day 92 were also maintained to day 169. This finding suggests that the beneficial effects of tralokinumab on lung function may be sustained for a prolonged period of at least 12 weeks after cessation of treatment.

This sustained effect on FEV₁ permits a dosing regimen of the invention that provides an initial “loading” first dosing period, followed by a subsequent “maintenance” second dosing period. In the loading first dosing period the dose may be administered at a higher dose than in the second maintenance period. In the loading first dosing period the dose may be administered more frequently than in the maintenance second dosing period. Accordingly, the invention provides a dosing regimen wherein the IL-13 antagonist (which is preferably an anti-human-IL-13 antibody, more preferably the anti-human IL-13 antibody is tralukinumab or a variant thereof or a human-IL-13 binding fragment thereof, as described herein) is administered:

(a) at a first dose at a first dosing interval for a first period of time, then at the first dose for a second dosing interval (which is longer than the first dosing interval) for a second period of time, for example by subcutaneous administration at 300 mg every two weeks (Q2W) for 8, 9, 10, 11, 12, 13, 14, 15, or 16 weeks followed by subcutaneous administration at 300 mg every 3, 4, 5, or 6 weeks (Q4W); preferably at 300 mg every two weeks (Q2W) for 12 weeks followed by subcutaneous administration at 300 mg every 4 weeks (Q4W); or,
(b) at a first dose at a first dosing interval for a first period of time, then at a second dose (which is lower than the first dose) at the first dosing interval, for example by subcutaneous administration at 300 mg every two weeks (Q2W) for 8, 9, 10, 11, 12, 13, 14, 15, or 16 weeks followed by subcutaneous administration at 150 mg every 2 weeks (Q2W); preferably at 300 mg every two weeks (Q2W) for 12 weeks followed by subcutaneous administration at 150 mg every 2 weeks (Q2W);
(c) at a first dose at a first dosing interval, then at a second dose (lower than the first dose) for a second dosing interval (which is longer than the first dosing interval), for example by subcutaneous administration at 300 mg every two weeks (Q2W) for 8, 9, 10, 11, 12, 13, 14, 15, or 16 weeks followed by subcutaneous administration at 150 mg every 3, 4, 5, or 6 weeks; preferably at 300 mg every two weeks (Q2W) for 12 weeks followed by subcutaneous administration at 150 mg every 4 weeks (Q4W).

When the IL-13 antagonist is Tralokinumab or a variant thereof or a human-IL-13 binding fragment thereof, the dosing regimen may be such that it is administered at 300 mg (e.g., as 2×150 mg subcutaneous injections every 2 weeks (Q2W) for 12 weeks followed by every 4 weeks (Q4W). Formulations of Tralokinumab suitable for use in such dosing regimens are described herein.

### FIGURE LEGENDS

FIG. 1: Study Flow Diagram: A Phase 2a, randomized, double-blinded placebo-controlled, parallel-arm study to evaluate the efficacy and safety of three subcutaneous treatment regimes of tralokinumab (CAT-354) in adult subjects with uncontrolled, moderate-to-severe, persistent asthma

FIG. 2a: Mean Change from Baseline in FEV₁ (Evaluable Population). Change from baseline in pre-bronchodilator FEV₁ to study day 92 for combined tralokinumab group and for placebo.

FIG. 2b: Change from baseline in FEV₁. Change from baseline in pre-bronchodilator FEV₁ to study day 92 for individual tralokinumab treatment groups (150 mg, 300 mg, or 600 mg administered subcutaneously every 2 weeks) and placebo.

FIG. 3: Change from baseline in pre-bronchodilator FEV₁ (L) at study day 92

FIG. 4: Change from baseline in pre-bronchodilator FEV₁ (L) at study day day 92 in subjects classified as atopic at baseline.

FIG. 5: Change from baseline in pre-bronchodilator FEV₁ at study day 92 in subjects classified as non-atopic at baseline.

FIG. 6: Change from baseline in % predicted pre-bronchodilator FEV₁ at study day 92

FIG. 7: Percentage of subjects showing a greater than, or equal to 10% change from baseline in pre-bronchodilator FEV₁

FIG. 8: Change from baseline in FVC at study day 92

FIG. 9: Decrease from baseline in SABA use at study day 92. Change in the mean number of “puffs” of short acting beta agonist (rescue treatment) taken daily comparing

FIG. 10a: Mean change from baseline in FEV₁ (Evaluable Population), change from baseline in pre-bronchodilator FEV₁ to study day 169 for combined tralokinumab group (150 mg, 300 mg, or 600 mg administered subcutaneously every 2 weeks) and for placebo.

FIG. 10b: Change from baseline in FEV₁, change from baseline in pre-bronchodilator FEV₁ to study day 169 for individual tralokinumab treatment groups (150 mg, 300 mg, or 600 mg administered subcutaneously every 2 weeks) and placebo.

FIG. 11: Mean (SD) change from baseline in ACQ-6 score over time (evaluable population) ACQ-6=Asthma Control Questionnaire, mean of 6 individual item scores.

FIG. 12: Mean (SD) change from baseline in pre-bronchodilator FEV₁ over time (evaluable population) FEV₁=forced expiratory volume in 1 s.

FIG. 13: Mean (SD) change from baseline in ACQ-6 score over time (evaluable population) in the subpopulation (n=56) who supplied a sputum sample at baseline. Placebo (sputum: all) is the subjects treated with placebo (n=17). Tralokinumab (sputum: all) is the subjects treated with tralokinumab (n=39). The tralokinumab-treated group (n=39) is subdivided into those without measurable IL-13 [tralokinumab (sputum IL-13<1 pg/mL)] and those with measurable IL-13 [tralokinumab (sputum IL-13≦1 pg/mL)]. ACQ-6=Asthma Control Questionnaire, mean of 6 individual item scores.

FIG. 14: Mean (SD) change from baseline in pre-bronchodilator FEV₁ over time (evaluable population) in the subpopulation (n=56) who supplied a sputum sample at baseline. Placebo (sputum: all) is the subjects treated with placebo (n=17). Tralokinumab (sputum: all) is the subjects treated with tralokinumab (n=39). The tralokinumab-treated group (n=39) is subdivided into those without measurable IL-13 [tralokinumab (sputum IL-13<1 pg/mL)] and those with measurable IL-13 [tralokinumab (sputum IL-13≦1 pg/mL)]. FEV₁=forced expiratory volume in 1 s.

### EXAMPLES

### Example 1

### Study Design

It was proposed that IL-13 blockade may reduce exacerbations and improve disease control (as defined by improvements in asthma symptoms coupled with reduced need for rescue medications) in patients whose asthma is uncontrolled, moderate-to-severe, and persistent despite maximal therapy at GINA step 4 or 5.

A Phase 2a, randomized, double-blind, placebo-controlled, parallel-arm study was performed to evaluate the efficacy and safety of three subcutaneous (SC) treatment regimens of tralokinumab, a recombinant human monoclonal antibody directed against interleukin 13 (IL-13), in adult subjects with uncontrolled, moderate-to-severe, persistent asthma. The study comprised a 2-week run-in period, a 12-week treatment period, and a 12-week follow-up period. Selection of dose regimen and treatment duration was based on pharmacokinetic modelling and simulations using data from previous clinical studies. This study is registered with ClinicalTrials.gov, number NCT00873860.

A total of 192 subjects were planned to be randomized. Prior to being randomized, subjects were stratified according to atopic asthma status using the Phadiatop test (Laboratoire Pasteur Cerba, France, a fluorescence enzyme immunoassay (FEIA) for allergen-specific immunoglobulin E (IgE) to the most frequent aero-allergens (mites, moulds, animals, grasses, weeds and trees) which determines presence of specific IgE antibodies to multiple inhaled allergens). Within each stratum, subjects were randomised according to a computer-generated randomisation list (1:1:1) to one of three arms (150 mg tralokinumab or placebo, 300 mg tralokinumab or placebo, 600 mg tralokinumab or placebo). Within each arm, subjects were then randomised according to a computer-generated randomisation list to active treatment or placebo (3:1). Study treatment was administered every 2 weeks by SC injection in the anterior thigh. Subjects received one, two or four SC injections (150 mg/l mL per injection) of tralokinumab at 7 visits over 12 weeks. All subjects were followed for 12 weeks after the last dose of investigational product (Study Day 169). Study medication was dispensed and administered by unblinded personnel, not otherwise involved in the study. Subjects continued their pre-study asthma controller therapy, as prescribed by their physician, during the study. Subjects and physicians were blinded to treatment allocation throughout.

An objective of this study was to evaluate the effect of the three (SC) treatment regimens of tralokinumab on asthma control at Study Day 92 versus placebo in adults with uncontrolled, moderate-to-severe, persistent asthma. The primary endpoint of the study was the Asthma Control Questionnaire (ACQ-6) (Juniper et al. (1999). Measures of pulmonary function, rescue medication use, asthma exacerbation rate, patient-reported outcomes (PROs), and safety were secondary endpoints. The secondary objectives included evaluating the effect of SC tralokinumab on the variability of airflow obstruction using the parameters of forced expiratory volume within 1 second (FEV₁) and peak expiratory flow (PEF).

### Major Inclusion Criteria

Male or female subjects.

Age 18 to 65 years at the time of screening.

Subjects must have a body mass index (BMI) between 18 and 40 kg/m².

Written informed consent obtained from the subject prior to performing any protocol-related procedures, including screening evaluations.

Physician-diagnosed moderate-to-severe, persistent asthma requiring treatment with appropriate asthma controller medication.

Shows FEV₁ reversibility post-bronchodilator of ≧12% and ≧200 mL or have shown such values in a previous test within the last year, or have a positive airway hyper-responsiveness (AHR) test result in the last year.

Pre-bronchodilator FEV₁ value ≧40% of individual predicted value at Visits 1 and 3.

Uncontrolled asthma consistent with Expert Panel Report (EPR)-3. In the 2 to 4 weeks preceding the screening visit, subjects should have a history of one or more of the following:

Daytime asthma symptoms ≧2 days/week
Night-time awakening ≧1 night/week
Salbutamol use ≧2 days/week.

An ACQ score 1.5 at Visits 1 and 3.

At least one occurrence of asthma exacerbation in the past year that required an unscheduled medical encounter.

Women of childbearing potential, unless surgically sterile (including tubal ligation) and/or at least 2 years postmenopausal must have a negative pregnancy test prior to the first dose of investigational product, and must agree to use 2 effective methods of avoiding pregnancy (including oral, injectable, transdermal, or implanted contraceptives, intrauterine device, female condom with spermicide, diaphragm with spermicide, cervical cap, use of condom by male sexual partner); or abstinence or sterile sexual partner from screening through Study Day 169. Women of childbearing potential must continue to practice birth control during the study and for at least 2 months after completing the study.

Men, unless surgically sterile, must likewise practice 2 effective methods of birth control (condom with spermicide) and must use such precautions from Study Day 1 through Study Day 169.

Otherwise healthy by medical history and physical examination for that age group.

A chest x-ray within the previous 12 months with no findings suggestive of acute or chronic respiratory pathology other than asthma.

Ability and willingness to complete the follow-up period until Study Day 169 as required by the protocol.

### Major Exclusion Criteria

Known history of allergy or reaction to any component of the investigational product formulation.

Acute illness other than asthma at the start of the study.

History of an active infection within 4 weeks prior to screening, or evidence of clinically significant active infection, including ongoing chronic infection. History of ingestion of untreated water in a location known to be infected with parasites, resulting in acute or chronic diarrhea; or a diagnosis of parasitic infection within 6 months prior to screening.

Use of immunosuppressive medication (except oral prednisone up to 10 mg/day and inhaled and topical corticosteroids) within 30 days before randomization into the study.

Receipt of immunoglobulin or blood products within 30 days before randomization into the study.

Receipt of any investigational drug therapy or use of any biologicals including omalizumab within 6 months before the first dose of investigational product in this study or within 5 half-lives of an investigational agent or biologic, whichever is longer.

History of any known immunodeficiency disorder.

A positive hepatitis B surface antigen, or hepatitis C virus antibody, as determined by medical history and/or subject's verbal report.

A positive human immunodeficiency virus test or is taking antiretroviral medications, as determined by medical history and/or subject's verbal report.

A live or attenuated vaccination received within 4 weeks prior to screening

Previous medical history, or evidence, of an intercurrent illness that may compromise the safety of the subject in the study.

History of clinically significant abnormality on electrocardiogram (ECG) in the opinion of the investigator.

### Lactation (Women)

History of treatment for alcohol or drug abuse within the past year.

History suggestive of chronic obstructive pulmonary disease (COPD) and of cigarette smoking≧10 pack-years.

Evidence of any systemic disease on physical examination.

History of cancer, apart from basal cell carcinoma or in situ carcinoma of the cervix treated with apparent success with curative therapy≦1 year prior to Study Day 1 or other malignancies treated with apparent success with curative therapy≦5 years prior to entry.

Known exposure to inhaled occupational agents or fumes.

Any condition (eg, cystic fibrosis [CF] or COPD) that, in the opinion of the investigator, would interfere with evaluation of the investigational product or interpretation of study results.

### Individuals Who are Legally Institutionalized

Employees of the clinical study site or any other individuals involved with the conduct of the study, or family members of such individuals.

### Target Patient Population

The subjects in this study were adult male and female subjects, 18 to 65 years of age, with uncontrolled, moderate-to-severe, persistent asthma.

### Primary Endpoint

The ACQ-6, a composite measure of asthma symptoms (night-time waking, symptoms on waking, activity limitation, shortness of breath, wheezing, and short-acting β₂-agonist use), was completed weekly using an electronic diary (eDiary). The primary endpoint was the change from baseline to week 13 in mean ACQ-6 score. Reductions in mean ACQ-6 score of 0.5 are considered to be clinically meaningful whilst a score of 1.5 indicates uncontrolled asthma (Juniper et al. (2006)).

### Secondary Endpoints

Spirometry was performed at every study visit in the morning; pre-bronchodilator FEV₁, forced vital capacity (FVC) and peak expiratory flow (PEF) were measured. Subjects also performed peak flow testing every morning at home.

For the purposes of the study, an asthma exacerbation was defined as a deterioration that required the administration of a systemic corticosteroid burst; specifically in a more detailed definition for the purpose of this study an asthma exacerbation (relapse or de novo) was defined as either a progressive increase of asthma symptoms (cough, wheeze, chest tightness, and/or shortness of breath) or a reduction of 20% in peak expiratory flow (PEF) or forced expiratory volume in 1 s (FEV₁) from baseline that did not resolve after the initiation of rescue medications and resulted in an administration of systemic corticosteroids by the investigator or healthcare provider. For those receiving maintenance oral corticosteroids (OCS) for the control of asthma, an asthma exacerbation was defined as occurring if OCS burst therapy or intravenous corticosteroids were initiated. Asthma exacerbations, if any, were reported at each study visit.

PROs recorded included a 4-item Daily Asthma Symptom Score and the Asthma Quality of Life Questionnaire with standardised activities (AQLQ(S)).

Adverse events (AEs), if any, were recorded at every study visit; routine laboratory evaluations were done at screening, after the first, third, and fifth doses, and at weeks 13 and 24.

Sputum was induced and collected at selected study centres at baseline, and weeks 8 and 14. Sputum IL-13 content was assessed using an ELISA system (Luminex® Corp).

### Pharmacokinetic Assessments

Blood samples were collected prior to each dose and during the follow-up period for pharmacokinetic assessment. Trough serum concentrations (Cₘᵢₙ) of tralokinumab were determined using an immunoassay performed on the Gyrolab assay platform that detects free tralokinumab. This method has a lower limit of quantification in serum of 0.300 pg/mL, and exhibits accuracy of <25% absolute relative error and precision of <20% CV (coefficient of variation).

Based on trough serum concentrations observed during the 12-week treatment period, exposure to tralokinumab increased approximately proportionally to dose over the range 150-600 mg. Mean (SD) serum concentrations at week 13 were 42.0 (16.3), 86.8 (33.1), and 156 (46.6) mg/mL for 150, 300, and 600 mg tralokinumab, respectively. Within each dose group, serum concentrations increased approximately twofold between weeks 2 and 10, consistent with moderate accumulation. Comparable serum concentrations were observed at weeks 10 and 12, indicating that steady-state had been attained by week 10.

### Immunogenicity

Blood samples were collected before the first dose and at weeks 13 and 24 for measurement of serum anti-drug antibodies using validated homogeneous bridging immunoassays with detection by electrochemiluminescence. No serum anti-drug antibodies were detected during the course of the study.

### Investigational Product, Dosage

A total of 192 subjects were planned to be randomized into this study, with 144 subjects to receive SC administration of Tralokinumab and 48 subjects to receive placebo. Tralokinumab was formulated at 150 mg/mL in 50 mM sodium acetate, 85 mM sodium chloride, 0.01% (w/v) plant-derived polysorbate 80 at pH 5.5)

Treatment Arm 1: 150 mg SC Tralokinumab (n=48) or 1 mL placebo (n=16) once every 2 weeks on Study Days 1, 15, 29, 43, 57, 71, and 85
Treatment Arm 2: 300 mg Tralokinumab (n=48) or 2 mL placebo (n=16) once every 2 weeks on Study Days 1, 15, 29, 43, 57, 71, and 85
Treatment Arm 3: 600 mg Tralokinumab (n=48) or 4 mL placebo (n=16) once every 2 weeks on Study Days 1, 15, 29, 43, 57, 71, and 85.

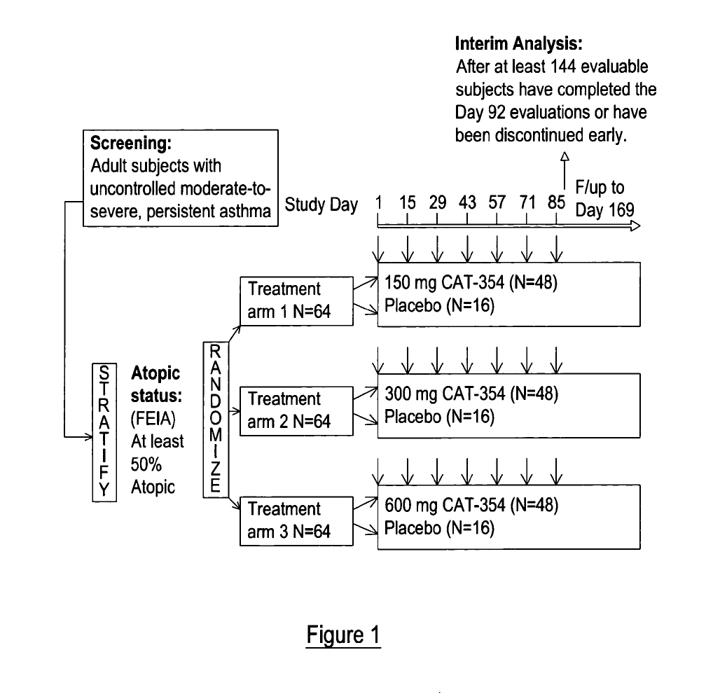

The study flow diagram is shown in FIG. 1. Five subject populations were analyzed in this study as follows.

1. Intent-to-treat (ITT) Population included all subjects who were randomized into the study.
2. Evaluable Population included all subjects who received at least 4 doses of investigational product as well as subjects who received at least one dose of investigational product but discontinued treatment prior to receiving 4 doses due to safety reasons. The primary efficacy analysis was based on the Evaluable Population.
3. According-to-Protocol (ATP) Population included all subjects who received all doses of investigational product and had an ACQ score at Study Day 92.
4. The Safety Population included all subjects who received at least one dose of investigational product.
5. The PK Population included all subjects in the Safety Population who received SC tralokinumab and had a sufficient number of serum concentration measurements.

TABLE 1
Summary of Subject Populations

| Subject | | SC TRALOKINUMAB | | | | |
|---|---|---|---|---|---|---|
| Populations | Placebo | 150 mg | 300 mg | 600 mg | Combined | Total |
| All Subjects | 48 | 47 | 51 | 48 | 146 | 194 |
| Randomized | | | | | | |
| ITT Population | 48 | 47 | 51 | 48 | 146 | 194 |
| Evaluable | 46 | 46 | 51 | 47 | 144 | 190 |
| Population | | | | | | |
| ATP | 44 | 45 | 47 | 44 | 136 | 180 |
| Population | | | | | | |
| Safety | 47 | 47 | 51 | 48 | 146 | 193 |
| Population | | | | | | |
| PK Population | 47 | 47 | 51 | 48 | 146 | 193 |

ATP = According-to-Protocol;
ITT = Intent-to-Treat;
PK = Pharmacokinetic.

FEV₁ was measured using the Masterscope® CT Spirometer using a protocol following the ATS/ERS recommendations 2005 (Eur Respir J; 26: 319-338).

### Statistical Methods

Sample size calculations based on the primary endpoint were conducted using statistical software nQuery Advisor® 6.01. Change from baseline in mean ACQ-6 score at week 13 was assumed to be −0.5 and −1.0 for the placebo and combined tralokinumab (150, 300, and 600 mg) groups, respectively, with a common SD of 0.9. A sample size of 144 was required to detect a statistically significant difference between combined tralokinumab groups and placebo at a 0.05 significance level with 80% power. To allow for dropouts it was planned to randomise 192 subjects.

The evaluable population included all subjects who received at least four doses of study medication and those who received at least one dose but discontinued prior to receiving four doses for safety reasons; the efficacy analyses reported are based on this population. The intent-to-treat (ITT) population was defined as all randomised subjects; confirmatory efficacy analysis was undertaken on this population. The safety population comprised all subjects who received at least one dose of study medication.

The primary endpoint was the comparison of the change from baseline to week 13 in mean ACQ-6 score between the combined tralokinumab group (i.e., 150, 300, and 600 mg) and placebo using analysis of variance. The primary efficacy analysis was undertaken at an interim analysis after all subjects had completed the week 13 visit. Two-sample t tests were used to compare between treatment group changes from baseline to week 13 in spirometry parameters measured during study visits. Efficacy endpoints were collected through to week 24 and were also analysed as described above (both study team and sites remained blinded to week 24). Exploratory efficacy analyses to compare each tralokinumab dose with placebo were undertaken and no multiple comparison adjustment was performed. Post-hoc analyses were conducted on the change from baseline to weeks 13 and 24 in rescue β₂-agonist use in the combined tralokinumab group and for each dose compared with placebo (two-sample t test). A post-hoc subgroup analysis defined by atopic asthma status was conducted on the change from baseline to week 13 in FEV₁.

A descriptive post-hoc analysis of the change from baseline in ACQ-6 and FEV₁ was undertaken in a subgroup of subjects with sputum samples obtained at baseline; subjects were analysed according to the presence or absence of detectable sputum IL-13.

Safety analysis was conducted after all subjects had completed the study using the safety population. All safety parameters were summarised descriptively.

### Results

The study was conducted between June 2009 and August 2010. 194 subjects were randomised at 27 study centres in Europe. Most subjects received all seven doses of study medication (placebo 95.7%; tralokinumab 93.8%).

### Disposition of Subjects

A total of 194 subjects was randomized in this Study at 27 sites in the European Union; 10 sites in Romania (n=77), 6 sites in Bulgaria (n=52), 6 sites in Poland (n=37), 4 sites in Germany (n=21), and one site in the United Kingdom (n=7).

Of the 194 subjects randomized in the study, 48 subjects were randomized to the placebo group and 146 subjects were randomized to one of three SC Tralokinumab treatment groups (47 subjects in the 150 mg SC

Tralokinumab treatment group, 51 subjects in the 300 mg SC Tralokinumab treatment group, and 48 subjects in the 600 mg SC Tralokinumab treatment group).

Demographic and Other Baseline Characteristics

Baseline demographic characteristics are summarized in Table 2. The mean age of the subject population was 47.3 years (18-65 years), with similar mean ages (43.4-49.8 years) seen across the placebo and SC tralokinumab treatment groups. The majority of subjects was female (59.8%), with the exception of the 150 mg SC tralokinumab treatment group (40.4%). Most subjects were White (91.8%). Mean BMI was 27.082 kg/m² (18.52-35.29 kg/m²), with similar mean BMI (26.548-27.536 kg/m²) seen across the placebo and SC tralokinumab treatment groups.

TABLE 2
Baseline Demographic Characteristics (ITT Population

| | | SC TRALOKINUMAB | | | | |
|---|---|---|---|---|---|---|
| | Placebo | 150 mg | 300 mg | 600 mg | Combined | Total |
| Parameters | (N = 48) | (N = 47) | (N = 51) | (N = 48) | (N = 146) | (N = 194) |
| Age (years) | | | | | | |
| Mean | 47.2 | 43.4 | 48.7 | 49.8 | 47.4 | 47.3 |
| SD | 9.8 | 11.1 | 11 | 10.4 | 11.1 | 10.8 |
| Median | 48 | 46 | 51 | 52 | 50 | 50 |
| Min-Max | (23-65) | (18-64) | (19-65) | (25-65) | (18-65) | (18-65) |
| Gender | | | | | | |
| Male | 15 | 28 | 15 (29.4%) | 20 | 63 | 78 |
| | (31.3%) | (59.6%) | | (41.7%) | (43.2%) | (40.2%) |
| Female | 33 | 19 | 36 (70.6%) | 28 | 83 | 116 |
| | (68.8%) | (40.4%) | | (58.3%) | (56.8%) | (59.8%) |
| Ethnicity | | | | | | |
| Hispanic or | 1 | 4 | 0 (0.0%) | 1 | 5 | 6 |
| Latino | (2.1%) | (8.5%) | | (2.1%) | (3.4%) | (3.1%) |
| Not | 47 | 43 | 51 (100%) | 47 | 141 | 188 |
| Hispanic or | (97.9%) | (91.5%) | | (97.9%) | (96.6%) | (96.9%) |
| Latino | | | | | | |
| Race | | | | | | |
| Asian | 1 | 0 | 1 (2.0%) | 0 | 1 | 2 |
| | (2.1%) | (0.0%) | | (0.0%) | (0.7%) | (1.0%) |
| White | 42 | 46 | 46 (90.2%) | 44 | 136 | 178 |
| | (87.5%) | (97.9%) | | (91.7%) | (93.2%) | (91.8%) |
| Other | 5 | 1 | 4 (7.8%) | 4 | 9 | 14 |
| | (10.4%) | (2.1%) | | (8.3%) | (6.2%) | (7.2%) |
| BMI (kg/m²) | | | | | | |
| Mean | 26.983 | 26.548 | 27.536 | 27.22 | 27.114 | 27.082 |
| SD | 4.001 | 3.777 | 4.181 | 3.876 | 3.949 | 3.952 |
| Median | 27.29 | 26.43 | 27.44 | 27.43 | 27.175 | 27.2 |
| Min-Max | (18.87-34.81) | (18.87-35.29) | (18.52-34.89) | (19.20-34.63) | (18.52-35.29) | (18.52-35.29) |

BMI = body mass index;
ITT = intent-to-treat;
Max = maximum;
Min = minimum;
SD = standard deviation.

Subjects with physician-diagnosed moderate-to-severe, persistent asthma requiring treatment with appropriate asthma controller medication were randomized in this study. Baseline disease characteristics and asthma medication history are summarized in Table 3 and Table 4, respectively.

At baseline, the study population had moderate-to-severe asthma that was not fully controlled (overall mean FEV₁ 2.00 L [61.2% predicted]; FVC 3.26 L; ACQ-6 score 2.66). Overall, mean FEV₁ was 61.2% (41%-103%) predicted, which was consistent across the placebo and SC tralokinumab treatment groups. Mean Asthma Symptom Score was 4.42 (0.0-8.1), with the majority (59.8%) of subjects (59.8%) having an average of >2 night-time awakenings per week. The majority (61.9%) of subjects also had at least one asthma exacerbation requiring oral corticosteroids (OCS) within the past 12 months. Stratification indicated that 47.9% of subjects had non-atopic asthma, whereas 52.1% of subjects were classified as atopic (defined by the presence of allergen-specific immunoglobulin E at the screening visit). Mean ACQ score was 2.66, which is consistent with an uncontrolled asthma population.

Subjects were taking a range of asthma controller medications at baseline and the mean inhaled corticosteroid (ICS) dose, expressed as beclomethasone dipropionate (BDP) equivalent, ranged from 1058.9-1254.3 mcg per day across the placebo and SC Tralokinumab treatment groups, the median daily ICS dose, expressed as beclomethasone diproprionate equivalent, was 1000 μg/day, 32% of subjects received high dose ICS (per GINA guideline 2009) and/or a regular daily dose of oral corticosteroid. Most subjects required rescue 132 agonists (mean 2.5 puffs/day) and short-acting beta agonists (90.7% using SABAs at least 3 days per week).

TABLE 3
Baseline Disease Characteristics (ITT Population)

| | | SC TRALOKINUMAB | | | | |
|---|---|---|---|---|---|---|
| | Placebo | 150 mg | 300 mg | 600 mg | Combined | Total |
| Parameters | (N = 48) | (N = 47) | (N = 51) | (N = 48) | (N = 146) | (N = 194) |
| Office | | | | | | |
| Spirometry | | | | | | |
| (FEV₁ % | | | | | | |
| Predicted) | | | | | | |
| N | 47 | 47 | 51 | 48 | 146 | 193 |
| Mean | 61.4 | 61.9 | 61.1 | 60.5 | 61.2 | 61.2 |
| SD | 12.3 | 12.7 | 12.6 | 11.8 | 12.3 | 12.3 |
| Median | 63.0 | 61.0 | 62.0 | 59.5 | 61.0 | 61.0 |
| Min-Max | (41-90) | (42-94) | (43-98) | (43-103) | (42-103) | (41-103) |
| FEV₁, L, mean | 1.94 | 2.20 | 1.90 | 1.96 | 2.01 | |
| (SD) | (0.48)* | (0.67) | (0.59) | (0.66) | (0.65) | |
| FVC, L, mean | 3.25 | 3.56 | 3.08 | 3.15 | 3.26 | |
| (SD) | (0.80)* | (0.95) | (1.01) | (1.04) | (1.02) | |
| Asthma Status | | | | | | |
| Non-atopic | 25 | 22 | 24 | 22 | 68 | 93 |
| | (52.1%) | (46.8%) | (47.1%) | (45.8%) | (46.6%) | (47.9%) |
| Atopic | 23 | 25 | 27 | 26 | 78 | 101 |
| | (47.9%) | (53.2%) | (52.9%) | (54.2%) | (53.4%) | (52.1%) |
| Asthma | | | | | | |
| Exacerbations | | | | | | |
| Requiring OCS | | | | | | |
| in Past 12 | | | | | | |
| Months | | | | | | |
| 0 | 18 | 18 | 23 | 15 | 56 | 74 |
| | (37.5%) | (38.3%) | (45.1%) | (31.3%) | (38.4%) | (38.1%) |
| 1 | 23 | 24 | 23 | 23 | 70 | 93 |
| | (47.9%) | (51.1%) | (45.1%) | (47.9%) | (47.9%) | (47.9%) |
| 2 | 6 | 4 | 3 | 6 | 13 | 19 |
| | (12.5%) | (8.5%) | (5.9%) | (12.5%) | (8.9%) | (9.8%) |
| 3 | 1 | 1 | 1 | 2 | 4 | 5 |
| | (2.1%) | (2.1%) | (2.0%) | (4.2%) | (2.7%) | (2.6%) |
| 5 | 0 | 0 | 0 | 1 | 1 | 1 |
| | (0.0%) | (0.0%) | (0.0%) | (2.1%) | (0.7%) | (0.5%) |
| PEF, L, mean | 284.9 | 341.2 | 290.5 | 309.9 | 313.2 | |
| (SD) | (79.6)* | (118.6) | (97.0) | (117.5) | (112.3) | |
| Rescue β₂- | 2.60 | 2.80 | 2.04 | 2.60 | 2.47 | |
| agonist use, | (1.68)* | (2.03) | (1.51) | (1.95) | (1.85) | |
| puffs/day, | | | | | | |
| mean (SD) | | | | | | |
| ACQ-6 score, | 2.60 | 2.68 | 2.63 | 2.72 | 2.68 | |
| mean (SD) | (0.54)* | (0.62) | (0.50) | (0.75) | (0.63) | |
| Overall | 4.15 | 3.90 | 4.15 | 4.16 | 4.07 | |
| AQLQ(S) score, | (0.83)† | (0.88)‡ | (0.80)‡ | (0.94)† | (0.87)§ | |
| mean (SD) | | | | | | |

ACQ = Asthma Control Questionnaire;
FEV₁ = Forced expiratory volume within 1 second;
Max = maximum;
Min = minimum;
N = number;
NA = not applicable;
SD = standard deviation.

TABLE 4
Summary of Asthma Medication History (ITT Population)

| | | SC TRALOKINUMAB | | | | |
|---|---|---|---|---|---|---|
| | Placebo | 150 mg | 300 mg | 600 mg | Combined | Total |
| Parameters | (N = 48) | (N = 47) | (N = 51) | (N = 48) | (N = 146) | (N = 194) |
| ICS Dose | | | | | | |
| (μg/day) | | | | | | |
| N | 45 | 46 | 50 | 48 | 144 | 189 |
| Mean | 1058.9 | 1254.3 | 1112.0 | 1254.2 | 1204.9 | 1170.1 |
| SD | 729.7 | 785.6 | 799.7 | 866.9 | 815.4 | 796.4 |
| Median | 1000.0 | 1000.0 | 1000.0 | 1000.0 | 1000.0 | 1000.0 |
| Min-Max | (200-4000) | (200-4000) | (100-4000) | (200-4000) | (100-4000) | (100-4000) |
| Subjects Who | 11 | 18 | 15 | 18 | 51 | 62 |
| Received | (22.9%) | (38.3%) | (29.4%) | (37.5%) | (34.9%) | (32.0%) |
| High-dose | | | | | | |
| ICS/OCS | | | | | | |
| Rescue β2 | | | | | | |
| Agonist Use | | | | | | |
| (Average | | | | | | |
| Number of | | | | | | |
| Puffs/Day) | | | | | | |
| N | 47 | 47 | 51 | 48 | 146 | 193 |
| Mean | 2.60 | 2.80 | 2.04 | 2.60 | 2.47 | 2.50 |
| SD | 1.68 | 2.03 | 1.51 | 1.95 | 1.85 | 1.81 |
| Median | 2.14 | 2.00 | 1.86 | 2.00 | 2.00 | 2.00 |
| Min-Max | (0.0-7.4) | (0.0-7.7) | (0.0-6.6) | (0.0-9.1) | (0.0-9.1) | (0.0-9.1) |
| SABA Use in | | | | | | |
| Last 3 | | | | | | |
| Months | | | | | | |
| 0-2 | 5 | 3 | 5 | 4 | 12 | 17 |
| Days/Week | (10.4%) | (6.4%) | (9.8%) | (8.3%) | (8.2%) | (8.8%) |
| 3-6 | 28 | 26 | 28 | 29 | 83 | 111 |
| Days/Week | (58.3%) | (55.3%) | (54.9%) | (60.4%) | (56.8%) | (57.2%) |
| 7 Days/Week | 15 | 18 | 17 | 15 | 50 | 65 |
| | (31.3%) | (38.3%) | (33.3%) | (31.3%) | (34.2%) | (33.5%) |
| NA | 0 | 0 | 1 | 0 | 1 | 1 |
| | (0.0%) | (0.0%) | (2.0%) | (0.0%) | (0.7%) | (0.5%) |

ICS = inhaled corticosteroids;
Max = maximum;
Min = minimum;
N = number;
NA = not applicable;
OCS = oral corticosteroids;
SABA = short-acting beta agonist;
SD = standard deviation.

### Efficacy Evaluation

This study was a Phase 2a study designed to establish whether blockade of IL-13 by SC Tralokinumab resulted in improved asthma control in subjects with uncontrolled, moderate-to-severe, persistent asthma. Efficacy results are reported for the evaluable population; results based on the ITT population were consistent with those for the evaluable population.

A 2-stage approach was used to analyze the data. Stage 1 (Study Day 92) of the analysis corresponded to the interim analysis specified by the protocol, i.e., the analysis of the primary endpoint was conducted after all subjects completed the Study Day 92 visit, with selected secondary endpoints included. Stage 2 (Study Day 169) corresponded to the final analysis specified in the protocol, i.e., the analysis of the overall study safety endpoints together with the remaining efficacy endpoints.

### Spirometry

Spirometry was performed at the study site during each visit. Multiple forced expiratory efforts (at least 3, but no more than 8) were performed for each spirometry session. The highest FEV₁ and FVC of all accepted efforts were used for the analysis. In addition, the PEF associated with the best effort of FEV₁ was used for analysis. The changes in airflow obstruction (FEV₁ and FVC at site and PEF at home) during the study were assessed.

Table 5 summarizes the change from baseline in spirometry parameters to Study Day 92. The mean change from baseline in FEV₁ at day 92 was 0.061

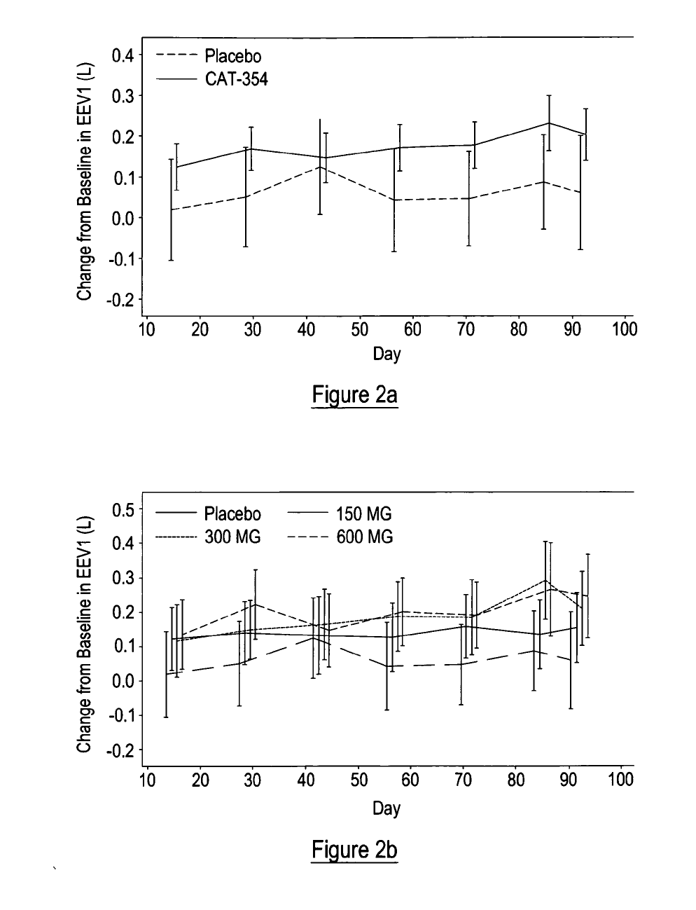

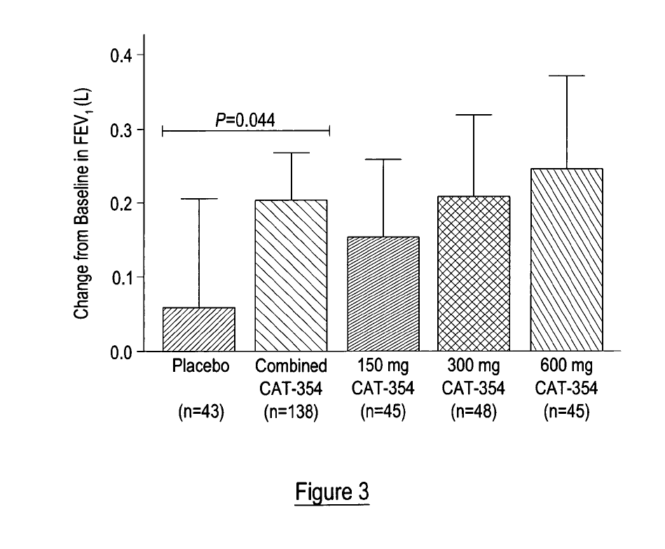

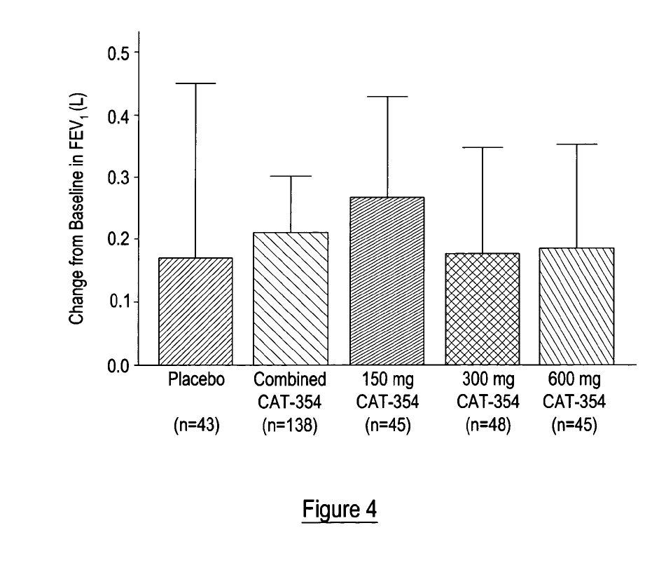

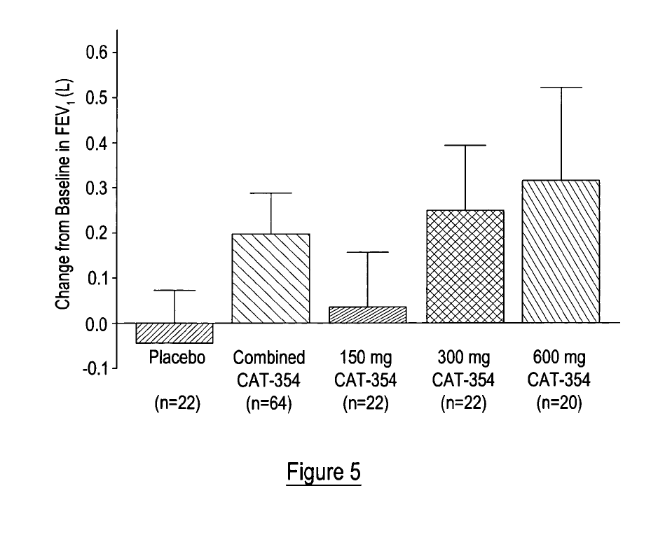

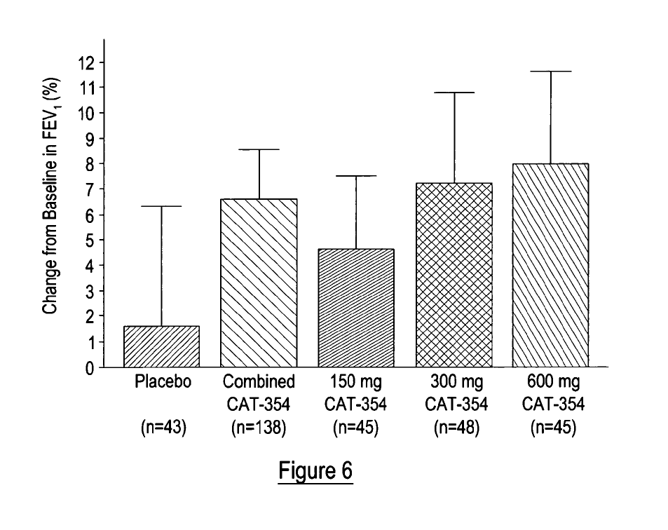

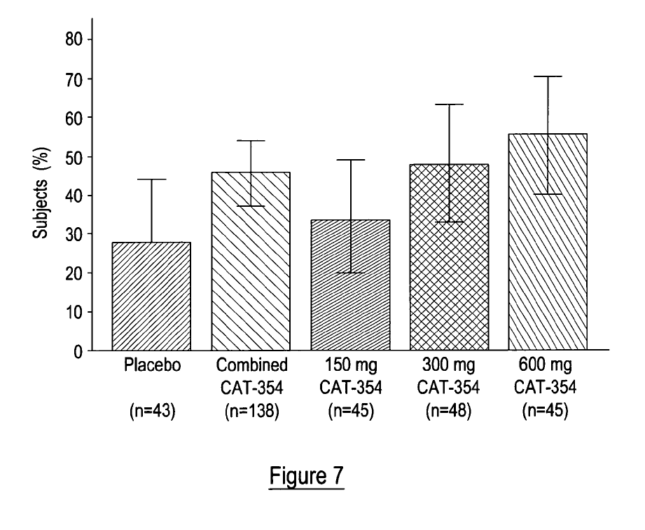

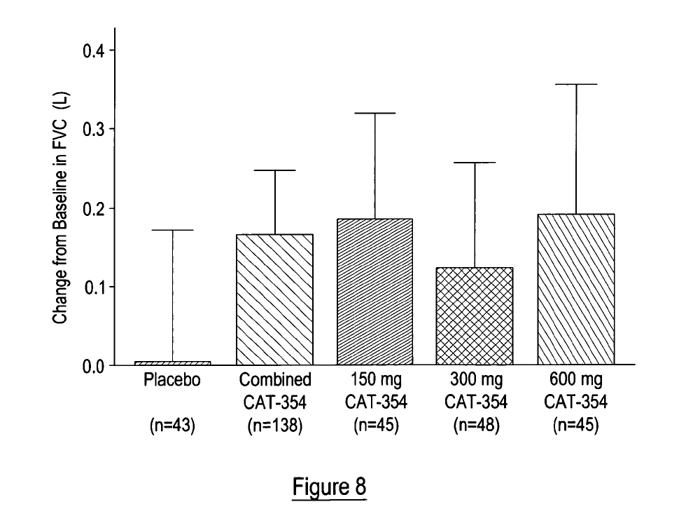

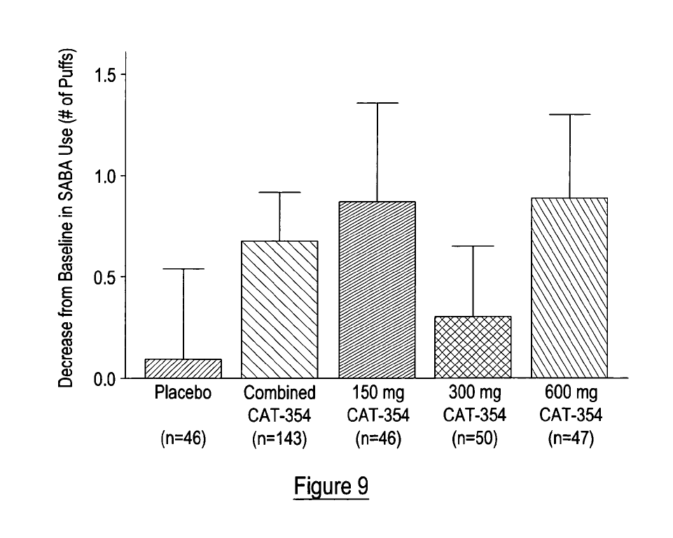

L in the placebo group and 0.204 L in the combined SC tralokinumab treatment group; this difference was statistically significant (p=0.044). This is shown graphically in FIG. 2. The increases in FEV₁ in the combined SC tralokinumab treatment group compared to placebo were observed as early as day 15 after the start of dosing, at this time point, the mean change from baseline in FEV₁ was 0.019 L in the placebo group and 0.124 L in the combined SC tralokinumab treatment group. At day 92, a dose response was seen across the SC tralokinumab treatment groups (0.155, 0.210, and 0.246 L, respectively). The mean percent changes in FEV₁ from baseline at Study Day 92 were 4.1% for the placebo group, 8.0% for the 150 mg SC tralokinumab treatment group, 13.3% for the 300 mg SC tralokinumab treatment group, 15.2% for the 600 mg SC tralokinumab treatment group, and 12.2% for the combined SC tralokinumab treatment group.

Sub-analyses were conducted to examine the effect of tralokinumab on FEV₁ in sub-populations within the study (Table 6). The magnitude of the mean change from baseline in FEV₁ in the combined SC tralokinumab treatment group was similar in subjects irrespective of their atopic status at baseline. For subjects with non-atopic asthma, the mean change from baseline in FEV₁ was 0.197 L in the combined SC tralokinumab treatment group (this difference was statistically significant compared to the placebo group (p=0.002). For subjects with atopic asthma, the mean change from baseline in FEV₁ was 0.210 L in the combined SC tralokinumab treatment group; (this difference was not statistically significant compared to the placebo group).

TABLE 5
Summary of change from baseline in spirometry parameters from baseline office visit evaluable population

| PARAMETER | TREATMENT | | | BASELINE | VISIT | CHANGE FROM BASELINE [1] | | | | |
|---|---|---|---|---|---|---|---|---|---|---|
| (UNIT) | GROUP | VISIT | N | MEAN | MEAN | MEAN | SD | MED | MIN | MAX |
| FEV1 (L) | PLACEBO (N = 46) | DAY 15 | 44 | 1.931 | 1.950 | 0.019 | 0.420 | −0.015 | −0.89 | 1.91 |
| | | DAY 29 | 46 | 1.947 | 1.998 | 0.051 | 0.424 | −0.005 | −0.48 | 2.03 |
| | | DAY 43 | 45 | 1.950 | 2.075 | 0.125 | 0.399 | 0.100 | −0.58 | 1.85 |
| | | DAY 57 | 45 | 1.956 | 2.000 | 0.043 | 0.436 | −0.060 | −0.93 | 1.87 |
| | | DAY 71 | 45 | 1.956 | 2.003 | 0.047 | 0.397 | 0.070 | −0.91 | 1.55 |
| | | DAY 85 | 45 | 1.956 | 2.043 | 0.087 | 0.397 | 0.020 | −0.59 | 1.86 |
| | | DAY 92 | 43 | 1.937 | 1.988 | 0.061 | 0.470 | 0.000 | −0.73 | 2.10 |
| | CAT-354 150 MG (N = 46) | DAY 15 | 46 | 2.178 | 2.300 | 0.122 | 0.316 | 0.065 | −0.40 | 1.07 |
| | | DAY 29 | 46 | 2.178 | 2.317 | 0.138 | 0.315 | 0.075 | −0.49 | 0.99 |
| | | DAY 43 | 44 | 2.205 | 2.336 | 0.131 | 0.380 | 0.075 | −0.45 | 1.92 |
| | | DAY 57 | 46 | 2.178 | 2.305 | 0.127 | 0.339 | 0.075 | −0.53 | 0.95 |
| | | DAY 71 | 45 | 2.191 | 2.349 | 0.158 | 0.314 | 0.090 | −0.32 | 0.80 |
| | | DAY 85 | 46 | 2.178 | 2.313 | 0.135 | 0.342 | 0.030 | −0.43 | 0.83 |
| | | DAY 92 | 45 | 2.168 | 2.322 | 0.155 | 0.345 | 0.130 | −0.39 | 1.21 |
| | CAT-354 300 MG (N = 51) | DAY 15 | 50 | 1.898 | 2.014 | 0.166 | 0.378 | 0.025 | −0.89 | 1.26 |
| | | DAY 29 | 49 | 1.907 | 2.056 | 0.149 | 0.303 | 0.110 | −0.46 | 1.09 |
| | | DAY 43 | 47 | 1.896 | 2.059 | 0.164 | 0.358 | 0.130 | −0.54 | 0.95 |
| | | DAY 57 | 48 | 1.890 | 2.078 | 0.188 | 0.353 | 0.120 | −0.44 | 1.04 |
| | | DAY 71 | 48 | 1.890 | 2.075 | 0.185 | 0.383 | 0.090 | −0.44 | 1.15 |
| | | DAY 85 | 47 | 1.890 | 2.181 | 0.291 | 0.395 | 0.230 | −0.31 | 1.28 |
| | | DAY 92 | 48 | 1.908 | 2.188 | 0.210 | 0.378 | 0.165 | −0.73 | 1.19 |
| | CAT-354 600 MG (N = 47) | DAY 15 | 46 | 1.996 | 2.130 | 0.134 | 0.348 | 0.080 | −0.51 | 1.52 |
| | | DAY 29 | 47 | 1.977 | 2.199 | 0.222 | 0.354 | 0.190 | −0.76 | 1.27 |
| | | DAY 43 | 47 | 1.977 | 2.124 | 0.147 | 0.373 | 0.070 | −0.43 | 1.53 |
| | | DAY 57 | 47 | 1.977 | 2.178 | 0.201 | 0.344 | 0.230 | −0.51 | 1.08 |
| | | DAY 71 | 47 | 1.977 | 2.176 | 0.190 | 0.335 | 0.130 | −0.65 | 0.99 |
| | | DAY 85 | 47 | 1.977 | 2.242 | 0.265 | 0.473 | 0.220 | −0.60 | 2.37 |
| | | DAY 92 | 45 | 1.952 | 2.198 | 0.246 | 0.418 | 0.260 | −0.55 | 1.74 |
| | CAT-354 TOTAL (N = 144) | DAY 15 | 142 | 2.020 | 2.144 | 0.124 | 0.347 | 0.065 | −0.89 | 1.52 |
| | | DAY 29 | 142 | 2.018 | 2.188 | 0.170 | 0.324 | 0.110 | −0.76 | 1.27 |
| | | DAY 43 | 138 | 2.022 | 2.170 | 0.148 | 0.368 | 0.090 | −0.54 | 1.92 |
| | | DAY 57 | 141 | 2.013 | 2.185 | 0.172 | 0.344 | 0.160 | −0.53 | 1.08 |
| | | DAY 71 | 140 | 2.016 | 2.194 | 0.178 | 0.344 | 0.120 | −0.65 | 1.15 |
| | | DAY 85 | 140 | 2.014 | 2.245 | 0.231 | 0.410 | 0.175 | −0.60 | 2.37 |
| | | DAY 92 | 138 | 2.007 | 2.211 | 0.204 | 0.380 | 0.150 | −0.73 | 1.74 |
| FVC (L) | PLACEBO (N = 46) | DAY 15 | 44 | 3.233 | 3.237 | 0.005 | 0.472 | −0.050 | −0.81 | 1.86 |
| | | DAY 29 | 46 | 3.261 | 3.260 | −0.001 | 0.504 | −0.090 | −0.63 | 2.11 |
| | | DAY 43 | 45 | 3.279 | 3.330 | 0.051 | 0.452 | 0.010 | −0.84 | 1.50 |
| | | DAY 57 | 45 | 3.264 | 3.279 | 0.015 | 0.531 | 0.000 | −0.92 | 2.15 |
| | | DAY 71 | 45 | 3.264 | 3.299 | 0.035 | 0.500 | −0.030 | −1.15 | 1.75 |
| | | DAY 85 | 45 | 3.264 | 3.320 | 0.056 | 0.493 | −0.050 | −0.70 | 2.03 |
| | | DAY 92 | 43 | 3.269 | 3.273 | 0.005 | 0.541 | −0.050 | −1.02 | 2.19 |
| | CAT-354 150 MG (N = 51) | DAY 15 | 46 | 3.534 | 3.626 | 00.92 | 0.371 | 0.100 | −0.65 | 0.99 |
| | | DAY 29 | 46 | 3.534 | 3.646 | 0.112 | 0.384 | 0.060 | −0.90 | 1.12 |
| | | DAY 43 | 44 | 3.558 | 3.710 | 0.152 | 0.503 | 0.115 | −0.61 | 2.54 |
| | | DAY 57 | 46 | 3.534 | 3.726 | 0.192 | 0.389 | 0.135 | −0.51 | 1.10 |
| | | DAY 71 | 45 | 3.535 | 3.694 | 0.158 | 0.432 | 0.040 | −0.62 | 1.15 |
| | | DAY 85 | 46 | 3.534 | 3.655 | 0.121 | 0.451 | 0.020 | −0.69 | 1.48 |
| | | DAY 92 | 45 | 3.536 | 3.723 | 0.187 | 0.448 | 0.140 | −0.71 | 1.81 |
| | CAT-354 300 MG (N = 51) | DAY 15 | 50 | 3.082 | 3.158 | 0.076 | 0.422 | 0.060 | −0.71 | 1.09 |
| | | DAY 29 | 49 | 3.107 | 3.185 | 0.077 | 0.300 | 0.080 | −0.66 | 0.87 |
| | | DAY 43 | 47 | 3.119 | 3.238 | 0.119 | 0.350 | 0.050 | −0.66 | 0.99 |
| | | DAY 57 | 48 | 3.094 | 3.210 | 0.116 | 0.309 | 0.065 | −0.52 | 0.83 |
| | | DAY 71 | 48 | 3.094 | 3.218 | 0.125 | 0.389 | 0.090 | −0.94 | 1.22 |
| | | DAY 85 | 47 | 3.106 | 3.316 | 0.210 | 0.431 | 0.170 | −0.78 | 1.57 |
| | | DAY 92 | 48 | 3.113 | 3.235 | 0.122 | 0.463 | 0.070 | −0.82 | 1.74 |
| | CAT-354 600 MG (N = 47) | DAY 15 | 46 | 3.206 | 3.310 | 0.105 | 0.482 | 0.040 | −0.92 | 1.40 |
| | | DAY 29 | 47 | 3.176 | 3.371 | 0.196 | 0.483 | 0.130 | −0.81 | 1.36 |
| | | DAY 43 | 47 | 3.176 | 3.341 | 0.166 | 0.553 | 0.050 | −1.14 | 1.82 |
| | | DAY 57 | 47 | 3.176 | 3.324 | 0.148 | 0.516 | 0.170 | −1.25 | 1.23 |
| | | DAY 71 | 47 | 3.176 | 3.344 | 0.168 | 0.544 | 0.040 | −1.59 | 1.47 |
| | | DAY 85 | 47 | 3.176 | 3.387 | 0.211 | 0.571 | 0.190 | −0.95 | 2.43 |
| | | DAY 92 | 45 | 3.139 | 3.330 | 0.191 | 0.554 | 0.220 | −1.71 | 1.71 |
| | CAT-354 TOTAL (N = 144) | DAY 15 | 142 | 3.269 | 3.359 | 0.090 | 0.424 | 0.070 | −0.92 | 1.40 |
| | | DAY 29 | 142 | 3.268 | 3.396 | 0.128 | 0.396 | 0.080 | −0.90 | 1.36 |
| | | DAY 43 | 138 | 3.278 | 3.424 | 0.145 | 0.473 | 0.065 | −1.14 | 2.54 |
| | | DAY 57 | 141 | 3.265 | 3.416 | 0.152 | 0.411 | 0.120 | −1.25 | 1.23 |
| | | DAY 71 | 140 | 3.263 | 3.413 | 0.150 | 0.457 | 0.055 | −1.59 | 1.47 |
| | | DAY 85 | 140 | 3.270 | 3.451 | 0.181 | 0.487 | 0.150 | −0.95 | 2.43 |
| | | DAY 92 | 138 | 3.259 | 3.425 | 0.166 | 0.487 | 0.135 | −1.71 | 1.81 |
| PEF (L/MIN) | PLACEBO (N = 46) | DAY 15 | 44 | 283.5 | 289.9 | 6.4 | 53.2 | −1.5 | −112 | 168 |
| | | DAY 29 | 46 | 284.4 | 291.8 | 7.4 | 49.0 | −4.5 | −58 | 181 |
| | | DAY 43 | 45 | 286.2 | 305.2 | 19.0 | 49.5 | 12.0 | −79 | 153 |
| | | DAY 57 | 45 | 287.2 | 294.5 | 7.2 | 53.6 | −4.0 | −122 | 172 |
| | | DAY 71 | 45 | 287.2 | 299.9 | 12.7 | 60.0 | 13.0 | −123 | 174 |
| | | DAY 85 | 45 | 287.2 | 301.7 | 14.5 | 47.6 | 7.0 | −89 | 171 |
| | | DAY 92 | 43 | 285.9 | 299.2 | 13.4 | 59.7 | 1.0 | −100 | 202 |
| | CAT-354 150 MG (N = 46) | DAY 15 | 46 | 338.3 | 353.5 | 15.2 | 51.0 | 7.0 | −82 | 129 |
| | | DAY 29 | 46 | 338.3 | 357.4 | 19.1 | 54.3 | 13.5 | −90 | 136 |
| | | DAY 43 | 44 | 345.5 | 364.3 | 18.8 | 59.1 | 11.5 | −89 | 221 |
| | | DAY 57 | 46 | 338.3 | 361.0 | 22.7 | 57.3 | 16.5 | −72 | 164 |
| | | DAY 71 | 45 | 341.5 | 372.8 | 31.3 | 59.8 | 17.0 | −97 | 157 |
| | | DAY 85 | 46 | 338.3 | 365.5 | 27.2 | 65.0 | 17.0 | −101 | 160 |
| | | DAY 92 | 45 | 339.2 | 367.4 | 28.2 | 60.6 | 34.0 | −89 | 149 |
| | CAT-354 300 MG (N = 51) | DAY 15 | 50 | 289.7 | 307.0 | 17.2 | 58.1 | 7.5 | −170 | 183 |
| | | DAY 29 | 49 | 293.5 | 311.4 | 17.9 | 61.7 | 13.0 | −123 | 206 |
| | | DAY 43 | 47 | 292.1 | 308.4 | 16.3 | 58.7 | 10.0 | −105 | 203 |
| | | DAY 57 | 48 | 289.0 | 310.3 | 21.3 | 60.3 | 5.0 | −93 | 201 |
| | | DAY 71 | 48 | 289.0 | 313.1 | 24.1 | 62.8 | 23.0 | −78 | 211 |
| | | DAY 85 | 47 | 289.3 | 332.7 | 43.4 | 63.7 | 23.0 | −56 | 237 |
| | | DAY 92 | 48 | 292.1 | 322.4 | 30.3 | 62.1 | 20.5 | −127 | 194 |
| | CAT-354 600 MG (N = 47) | DAY 15 | 46 | 316.8 | 340.7 | 23.8 | 57.8 | 25.5 | −81 | 214 |
| | | DAY 29 | 47 | 314.0 | 349.0 | 35.0 | 61.1 | 27.0 | −94 | 197 |
| | | DAY 43 | 47 | 314.0 | 331.8 | 17.8 | 58.8 | 16.0 | −98 | 181 |
| | | DAY 57 | 47 | 314.0 | 340.1 | 26.1 | 57.0 | 32.0 | −102 | 141 |
| | | DAY 71 | 47 | 314.0 | 347.7 | 33.7 | 59.9 | 31.0 | −94 | 164 |
| | | DAY 85 | 47 | 314.0 | 350.3 | 36.3 | 77.5 | 26.0 | −114 | 313 |
| | | DAY 92 | 45 | 311.8 | 347.0 | 35.2 | 66.7 | 29.0 | −97 | 237 |

[1] CHANGE FROM BASELINE = CURRENT VISIT VALUE − BASELINE VALUE

TABLE 6
Change from baseline in FEV1 and PEF from office visit at day 92 [1] Evaluable population

| | | | | CAT-354 | CAT-354 | CAT-354 | CAT-354 |
|---|---|---|---|---|---|---|---|
| | | | PLACEBO | 150 MG | 300 MG | 600 MG | TOTAL |
| | | | (N = 46) | (N = 46) | (N = 51) | (N = 47) | (N = 144) |
| FEV1 (L) | ALL | N | 43 | 45 | 48 | 45 | 138 |
| | | MEAN | 0.061 | 0.155 | 0.210 | 0.246 | 0.204 |
| | | SD | 0.470 | 0.345 | 0.378 | 0.418 | 0.380 |
| | | MEDIAN | 0.000 | 0.130 | 0.165 | 0.260 | 0.150 |
| | | MIN-MAX | (−0.73-2.10) | (−0.39-1.21) | (−0.73-1.19) | (−0.55-1.74) | (−0.73-1.74) |
| | | P VALUE [2] | | 0.289 | 0.097 | 0.055 | 0.044 |
| | SUBJECTS WITH | N | 21 | 23 | 26 | 25 | 74 |
| | ATOPIC ASTHMA | MEAN | 0.172 | 0.268 | 0.178 | 0.189 | 0.210 |
| | | SD | 0.607 | 0.371 | 0.414 | 0.400 | 0.393 |
| | | MEDIAN | 0.060 | 0.150 | 0.140 | 0.170 | 0.155 |
| | | MIN-MAX | (−0.65-2.10) | (−0.30-1.21) | (−0.73-1.19) | (−0.55-1.07) | (−0.73-1.21) |
| | | P VALUE [2] | | 0.527 | 0.970 | 0.913 | 0.737 |
| | SUBJECTS WITH | N | 22 | 22 | 22 | 20 | 64 |
| | NON-ATOPIC ASTHMA | MEAN | −0.045 | 0.036 | 0.249 | 0.317 | 0.194 |
| | | SD | 0.259 | 0.277 | 0.335 | 0.439 | 0.368 |
| | | MEDIAN | −0.020 | 0.000 | 0.180 | 0.290 | 0.135 |
| | | MIN-MAX | (−0.73-0.54) | (−0.39-0.89) | (−0.28-1.04) | (−0.43-1.74) | (−0.43-1.74) |
| | | P VALUE [2] | | 0.323 | 0.002 | 0.002 | 0.006 |
| | SUBJECTS IN LOWER | N | 23 | 19 | 24 | 20 | 63 |
| | TERTILE OF BASELINE | MEAN | 0.127 | 0.182 | 0.168 | 0.266 | 0.203 |
| | MEAN ACQ | SD | 0.568 | 0.301 | 0.339 | 0.308 | 0.316 |
| | | MEDIAN | 0.050 | 0.150 | 0.140 | 0.370 | 0.210 |
| | | MIN-MAX | (−0.73-2.10) | (−0.35-0.89) | (−0.73-0.97) | (−0.43-0.67) | (−0.73-0.97) |
| | | P VALUE [2] | | 0.708 | 0.764 | 0.355 | 0.434 |
| | SUBJECTS IN MIDDLE | N | 8 | 10 | 12 | 13 | 35 |
| | TERTILE OF BASELINE | MEAN | 0.074 | 0.165 | 0.269 | 0.141 | 0.192 |
| | MEAN ACQ | SD | 0.260 | 0.299 | 0.457 | 0.357 | 0.374 |
| | SUBJECTS IN MIDDLE | MEDIAN | 0.005 | 0.125 | 0.200 | 0.130 | 0.140 |
| | TERTILE OF BASELINE | MIN-MAX | (−0.23-0.54) | (−0.27-0.71) | (−0.57-1.04) | (−0.46-1.07) | (−0.57-1.07) |
| | MEAN ACQ | P VALUE[2] | | 0.506 | 0.290 | 0.652 | 0.404 |
| | SUBJECTS IN UPPER | N | 12 | 16 | 12 | 12 | 40 |
| | TERTILE OF BASELINE | MEAN | −0.073 | 0.116 | 0.237 | 0.326 | 0.215 |
| | MEAN ACQ | SD | 0.358 | 0.429 | 0.390 | 0.611 | 0.476 |
| | | MEDIAN | −0.140 | 0.030 | 0.135 | 0.140 | 0.075 |
| | | MIN-MAX | (−0.65-0.66) | (−0.39-1.21) | (−0.12-1.19) | (−0.55-1.74) | (−0.55-1.74) |
| | | P VALUE [2] | | 0.260 | 0.055 | 0.064 | 0.059 |
| | SUBJECTS WITH % OF | N | 19 | 22 | 22 | 24 | 68 |
| | PREDICTED BASELINE | MEAN | 0.154 | 0.128 | 0.289 | 0.378 | 0.268 |
| | FEV1 <=60% | SD | 0.596 | 0.326 | 0.322 | 0.446 | 0.381 |
| | | MEDIAN | 0.030 | 0.130 | 0.210 | 0.285 | 0.200 |
| | | MIN-MAX | (−0.65-2.10) | (−0.35-0.89) | (−0.22-1.04) | (−0.46-1.74) | (−0.46-1.74) |
| | | P VALUE [2] | | 0.858 | 0.364 | 0.116 | 0.316 |
| | SUBJECTS WITH HIGH | N | 11 | 17 | 13 | 16 | 46 |
| | DOSE OF ICS OR OCS | MEAN | 0.209 | 0.182 | 0.122 | 0.313 | 0.186 |
| | AT BASELINE | SD | 0.756 | 0.301 | 0.455 | 0.481 | 0.435 |
| | | MEDIAN | −0.030 | 0.150 | 0.000 | 0.370 | 0.115 |
| | | MIN-MAX | (−0.73-2.10) | (−0.35-0.89) | (−0.39-1.21) | (−0.28-1.04) | (−0.55-1.74) |
| | | P VALUE [2] | | 0.708 | 0.675 | 0.729 | 0.894 |

[1] CHANGE FROM BASELINE = CURRENT VISIT VALUE − BASELINE VALUE
[2] P-VALUES ARE CALCULATED USING AN ANALYSIS OF VARIANCE REFER TO SUPPORTING DATA LISTING(S) 16.

The magnitude of the mean change from baseline in FEV₁ of 0.186 L in subjects receiving high dose inhaled corticosteroids and/or oral corticosteroids as background asthma controller therapy within the combined SC tralokinumab treatment group was similar compared to the overall study population (this difference was not statistically significant compared to the placebo group).

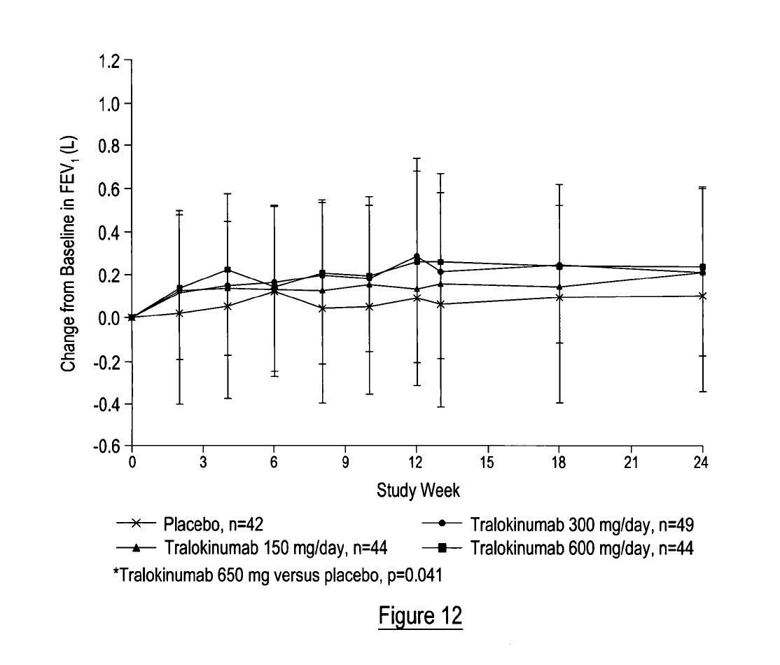

Pre-bronchodilator FEV₁, FVC, and PEF improved in all tralokinumab groups over the 12-week treatment period compared with placebo (table 8). The mean [SD] percent increases from baseline to week 13 in FEV₁ (12.5% [20.7%] vs 4.3% [29.4%]) and FVC (7.1% [16.8%] vs 1.5% [21.0%]) were greater for tralokinumab versus placebo. The effect on FEV₁ was evident at 2 weeks and persisted until week 24 (FIG. 12). At week 13 a dose response was evident, though qualitatively similar FEV₁ increases were observed in the tralokinumab 300 and 600 mg groups during the last 6 weeks of treatment. Similar mean [SD] percent increases in FEV₁ were seen at week 13 in subjects with atopic (12.9% [21.2%]) and non-atopic (12.2% [20.2%]) asthma who received tralokinumab.

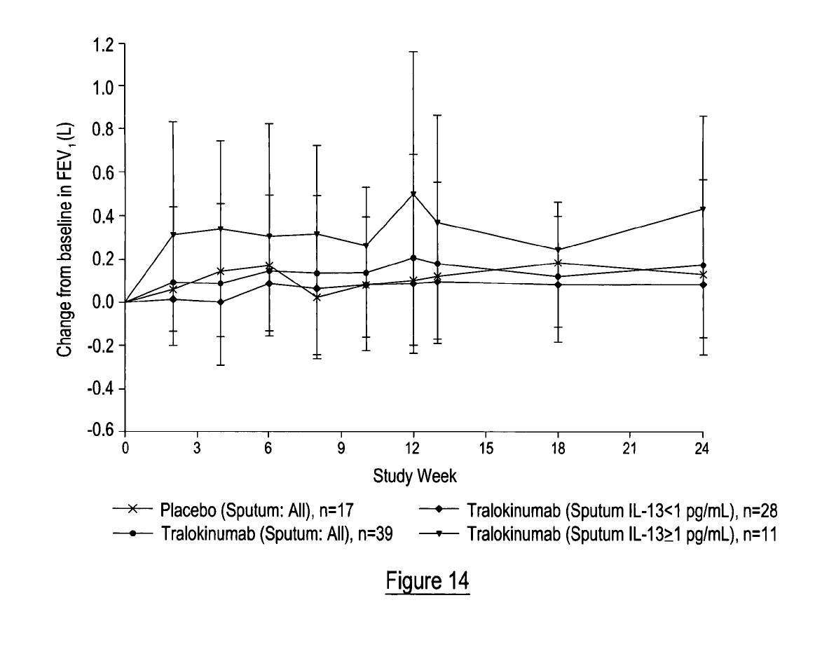

Changes in FEV₁ in those subjects providing a sputum sample at baseline, stratified by the presence or absence of IL-13, are presented in FIG. 14. The week 13 mean [SD] improvement in the sputum IL-13-positive (lpg/mL) tralokinumab group (9.9 [11.4]% n=11) was greater than placebo (3.9 [9.0]% n=15) or sputum IL-13-negative (<1 pg/mL) tralokinumab group (2.9 [8.9]% n=26).

Rescue 132 Agonist Use Table 7 summarizes the use of rescue beta2 agonist and the proportion of reliever-free days comparing the week preceding baseline and the week preceding Study Day 92 across the placebo and SC tralokinumab treatment groups. The mean number of rescue beta2 agonist puffs was similar at both time points in the placebo group (2.58 puffs at baseline and 2.5 puffs at Study Day 92). In contrast, the mean number of rescue beta2 agonist puffs was lower at Study Day 92 as compared to baseline in the combined SC tralokinumab treatment group (2.48 puffs at baseline and 1.8 puffs at Study Day 92).

The mean proportion of reliever-free days in a week in the placebo group was 13.2% at baseline and 22.0% at Study Day 92 for a difference of 8.8%. In the combined SC tralokinumab treatment group, the mean proportion of reliever-free days in a week was 17.4% at baseline and 38.7% at Study Day 92 for a difference of 21.3%.

Subjects in the combined tralokinumab group showed a significantly greater mean [SD] reduction in β₂-agonist use compared with placebo at week 13 (−0.68 [1.45] vs −0.10 [1.49] puffs/day; p=0.020) and week 24 (−0.73 [1.57] vs −0.05 [1.87] puffs/day; p=0.025). The mean proportion of reliever-free days/week increased during the study in the combined tralokinumab versus placebo groups (174% vs 13.2%, 38.7% vs 22.0%, 38.6% vs 28.6% at baseline, week 13, week 24, respectively).

TABLE 7
Summary of Rescue Beta2 Agonist Use and Reliever-free Days at
Baseline and at Study Day 92 (Evaluable Population)

| | | SC TRALOKINUMAB | | | |
|---|---|---|---|---|---|
| | Placebo | 150 mg | 300 mg | 600 mg | Combined |
| Parameter | (N = 46) | (N = 46) | (N = 51) | (N = 47) | (N = 144) |
| Rescue Beta2 | | | | | |
| Agonist Use | | | | | |
| (Average | | | | | |
| Number | | | | | |
| of Puffs) | | | | | |
| Baseline | | | | | |
| N | 46 | 46 | 51 | 47 | 144 |
| Mean | 2.58 | 2.84 | 2.04 | 2.62 | 2.48 |
| SD | 1.70 | 2.03 | 1.51 | 1.96 | 1.86 |
| Median | 2.14 | 2.07 | 1.86 | 2.00 | 2.00 |
| Min-Max | (0.0-7.4) | (0.0-7.7) | (0.0-6.6) | (0.0-9.1) | (0.0-9.1) |
| Study Days | | | | | |
| 86-92 | | | | | |
| N | 46 | 46 | 50 | 47 | 143 |
| Mean | 2.5 | 2.0 | 1.8 | 1.7 | 1.8 |
| SD | 2.3 | 2.1 | 2.0 | 1.7 | 2.0 |
| Median | 2.0 | 1.8 | 1.3 | 1.7 | 1.4 |
| Min-Max | (0-8) | (0-8) | (0-10) | (0-6) | (0-10) |
| Proportion of | | | | | |
| Reliever-free | | | | | |
| Days in a | | | | | |
| Week (%) | | | | | |
| Baseline | | | | | |
| N | 46 | 46 | 51 | 47 | 144 |
| Mean | 13.2 | 12.1 | 24.1 | 15.3 | 17.4 |
| SD | 24.8 | 25.5 | 34.6 | 28.1 | 30.1 |
| Median | 0.0 | 0.0 | 0.0 | 0.0 | 0.0 |
| Min-Max | (0-100) | (0-100) | (0-100) | (0-100) | (0-100) |
| Study Days | | | | | |
| 86-92 | | | | | |
| N | 46 | 46 | 50 | 47 | 143 |
| Mean | 22.0 | 37.3 | 40.6 | 38.0 | 38.7 |
| SD | 36.5 | 43.3 | 43.6 | 45.2 | 43.8 |
| Median | 0.0 | 0.0 | 28.6 | 0.0 | 0.0 |
| Min-Max | (0-100) | (0-100) | (0-100) | (0-100) | (0-100) |

Max = maximum;
Min = minimum;
N = number;
PEF = peak expiratory flow;
SD = standard deviation.

### Efficacy Conclusions

A statistically significant increase in FEV₁ from baseline was seen in the combined SC tralokinumab treatment group compared to placebo at Study Day 92. The magnitude of this change (0.204 L or 12.2%) is clinically important, particularly as it was observed in addition to currently available asthma controller medications. The treatment effect was observed irrespective of atopic status and also in subjects receiving high doses of inhaled corticosteroids and/or oral steroids.

The increase in FEV₁ appeared to be dose dependant, with the greatest increases seen in the 600 and 300 mg SC tralokinumab treatment groups.

Increases from baseline in PEF and FVC were also observed in the combined SC tralokinumab treatment group.

A reduction in the requirement for the use of additional short acting beta2 agonist was observed in the combined SC tralokinumab treatment group that is consistent with the improvement in FEV₁ described above.

Primary Endpoint—Change in ACQ-6 Score from Baseline to Week 13

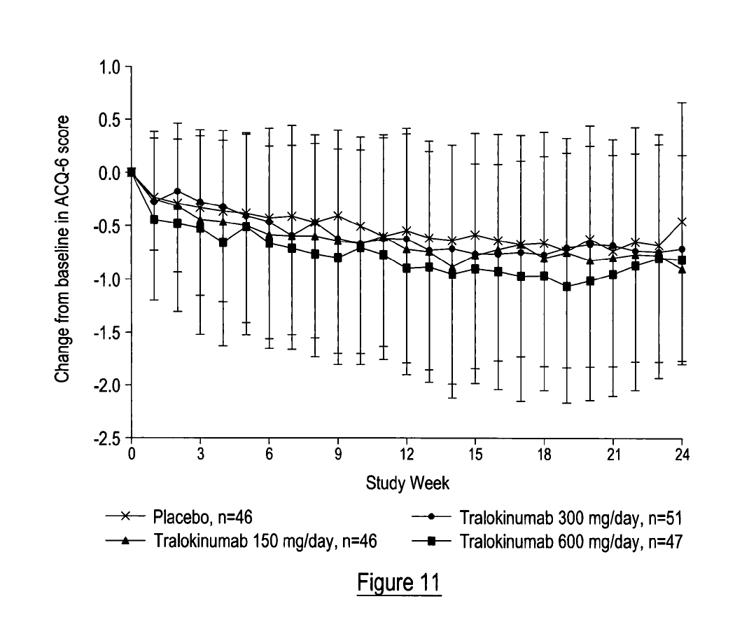

Mean ACQ-6 score improved from baseline to week 13 in all treatment groups (table 8; FIG. 11), with 84/144 (58.3%) tralokinumab and 24/46 (52.2%) placebo subjects showing improvement of 0.5. The mean [SD] reduction in the combined tralokinumab group (−0.76 [1.04]) at week 13 was not different from placebo (−0.61 [0.90]; p=0.375). The difference (95% CI) between treatment groups in change from baseline was −0.15 (−049, 0.19). Reductions in ACQ-6 score were maintained through week 24 in both groups although there was no difference in the mean [SD] between the tralokinumab (−0.79 [0.98]) and placebo (−0.60 [1.00]; p=0.256) groups.

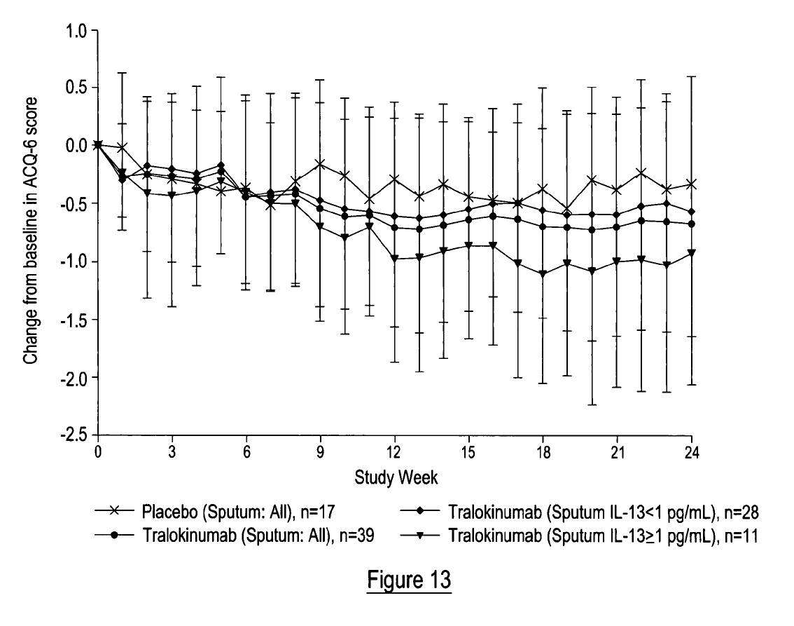

However, a difference was observed in ACQ-6 score in those subjects providing a sputum sample at baseline, stratified by the presence or absence of IL-13, this is presented in FIG. 13; the week 13 mean [SD] reduction in ACQ-6 in the sputum IL-13-positive (1 pg/mL) tralokinumab group (−0.97 [0.98] n=11) was greater than that in the placebo (−043 [0.70] n=17) or sputum IL-13-negative (<1 pg/mL) tralokinumab group (−0.62 [0.85] n=28).

TABLE 8
Summary of change from baseline in mean ACQ-6 score and
spirometry parameters at office visits at week 13 (evaluable
population)

| | Tralokinumab | | | |
|---|---|---|---|---|
| | | | | Combined |
| Placebo | 150 mg | 300 mg | 600 mg | tralokinumab |

Mean ACQ-6 score change from baseline

| | | | | | |
|---|---|---|---|---|---|
| n | 46 | 46 | 51 | 47 | 144 |
| Mean | −0.61 | −0.73 | −0.70 | −0.86 | −0.76 |
| (SD) | (0.90) | (1.12) | (0.93) | (1.09) | (1.04) |
| Difference | — | −0.12 | −0.09 | −0.25 | −0.15 |
| (95% CI)* | | (−0.54, | (−0.46, | (−0.66, | (−0.49, |
| | | 0.30) | 0.28) | 0.16) | 0.19) |
| p value† | — | 0.573 | 0.640 | 0.224 | 0.375 |

FEV₁ (L) change from baseline

| | | | | | |
|---|---|---|---|---|---|
| n | 42 | 44 | 49 | 44 | 137 |
| Mean (SD) | 0.06 | 0.16 | 0.21 | 0.26 | 0.21 |
| | (0.48) | (0.35) | (0.37) | (0.41) | (0.38) |
| Difference | — | 0.09 | 0.15 | 0.20 | 0.15 |
| (95% CI)* | | (−0.09, | (−0.03, | (0.01, | (−0.01, |
| | | 0.27) | 0.32) | 0.39) | 0.31) |
| p value‡ | — | 0.299 | 0.102 | 0.041 | 0.072 |
| Mean (SD) | 4.3 | 8.1 (17.5) | 13.3 | 16.1 | 12.5 |
| % change§ | (29.4) | | (21.8) | (22.0) | (20.7) |

FVC (L) change from baseline

| | | | | | |
|---|---|---|---|---|---|
| n | 42 | 44 | 49 | 44 | 137 |
| Mean (SD) | 0.00 | 0.18 | 0.12 | 0.21 | 0.17 |
| | (0.55) | (0.45) | (0.46) | (0.54) | (0.48) |
| Difference | — | 0.18 | 0.12 | 0.21 | 0.17 |
| (95% CI)* | | (−0.04, | (−0.09, | (−0.03, | (−0.01, |
| | | 0.39) | 0.33) | 0.44) | 0.34) |
| p-value‡ | — | 0.100 | 0.261 | 0.082 | 0.059 |
| Mean (SD) | 1.5 | 6.4 (15.0) | 5.5 | 9.6 (18.8) | 7.1 |
| % change§ | (21.0) | | (16.4) | | (16.8) |

PEF (L/min) change from baseline

| | | | | | |
|---|---|---|---|---|---|
| n | 42 | 44 | 49 | 44 | 137 |
| Mean (SD) | 14.2 | 27.5 | 30.0 | 38.2 | 31.8 |
| | (60.1) | (61.1) | (61.5) | (64.3) | (62.0) |
| Difference | — | 13.3 | 15.8 | 24.0 | 17.6 |
| (95% CI)* | | (−12.7, | (−9.7, | (−2.7, | (−3.8, |
| | | 39.3) | 41.2) | 50.7) | 39.0) |
| p value‡ | — | 0.313 | 0.222 | 0.078 | 0.107 |
| Mean (SD) | 5.4 | 11.0 | 14.1 | 16.5 | 13.9 |
| 13/0 change§ | (23.6) | (23.6) | (27.5) | (25.7) | (25.6) |

ACQ-6 = Asthma Control Questionnaire, mean of 6 individual item scores.
CI = confidence interval.
FEV₁ = forced expiratory volume in 1 s.
FVC = forced vital capacity.
PEF = peak expiratory flow.
SD = standard deviation.
*Difference in mean change from baseline: tralokinumab-placebo.
†p values were based on analysis of variance.
‡p values were calculated using a 2-sample t test.
§Change from baseline: current visit value-baseline value.

### Safety and Tolerability Conclusions

The majority of subjects received all 7 doses of investigational product and there was a low incidence of subjects dropping-out of the study. tralokinumab administered as a SC injection at doses of 150, 300, and 600 mg every other week for 12 weeks was generally well tolerated. No major safety issues were identified.

### Post-Treatment Follow-Up

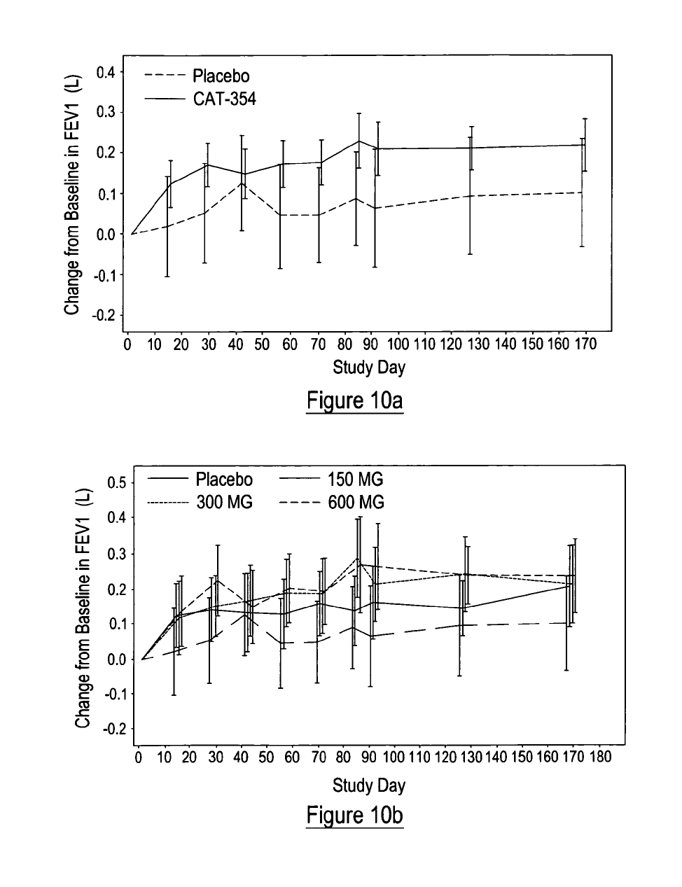

All subjects were followed for 12 weeks after the last dose of investigational product and underwent spirometry at days 127 and day 169. The increases in FEV₁ that were observed in tralokinumab-treated subjects at day 92 were maintained at both these post-doing time points. The mean change from baseline in FEV₁ at day 169 was 0.101 L in the placebo group and 0.218 L in the combined SC tralokinumab treatment group (p=0.096). The numerical increases in FVC and PEF observed at day 92 were also maintained to day 169. This finding suggests that the beneficial effects of tralokinumab on lung function may be sustained for a prolonged period after cessation of treatment (see FIGS. 10a & 10b).

### Summary

Evidence of clinical effect was observed following the addition of tralokinumab to standard asthma medications in subjects with uncontrolled moderate-to-severe persistent asthma. The most compelling signal was the statistically significant increase in FEV₁ at day 92 that was also supported by the improvements in FVC, PEF and reduction in the use of short-acting beta2 agonist. The improvement in airflow obstruction was observed as early as day 15 following randomisation. As regards differentiation between doses of tralokinumab, the largest treatment effects were observed in the 600 mg SC tralokinumab treatment group but clinically important changes in FEV₁ were also observed in the 300 mg SC tralokinumab treatment group. The improvement in FEV₁ in the tralokinumab treatment group was maintained in the 12-week post-treatment follow-up period. A sub-set of subjects stratified into sputum IL-13 positive or negative demonstrated an association between presence of IL-13 in sputum and response to tralokinumab as assessed by ACQ-6.

A growing body of evidence implicates IL-13 as a key mediator in the development and maintenance of asthma. This study investigated the additive effect of tralokinumab, a potent and specific IL-13-neutralising human IgG4 monoclonal antibody, to currently available asthma controller therapy in subjects with uncontrolled moderate-to-severe asthma. The increases in FEV₁ and FVC and reduction in the use of short-acting (3₂-agonists in tralokinumab-treated subjects are indicative of a treatment effect. The magnitude of increase in FEV₁ approximates the minimal important difference of 10% (Reddel et al., (2009)) suggesting that this antibody is able to deliver a clinically important benefit.

The use of tralokinumab resulted in a reduction in the need for ‘rescue’ short acting β agonists (SABA) that is consistent with the improvement observed in FEV₁. The largest effect size was observed in 600 mg treatment group. The beneficial effect on FEV₁ was observed irrespective of atopic status at baseline and also in subjects receiving high doses of inhaled corticosteroids and/or oral steroids and persisted for 12 weeks following the last treatment.

As asthma is a heterogeneous disease, identification of patient subgroups or individual patient/disease characteristics is important in delivering optimal benefit with biotherapeutics that, by design, target specific mechanisms. We postulated that subjects who have upregulated airway IL-13 may benefit most from treatment with IL-13-neutralising therapy. We explored this concept in our study by examining 56 subjects who supplied a baseline sputum sample. When subjects were stratified into sputum IL-13 positive or negative there was an association between presence of IL-13 and response to tralokinumab (FIGS. 13 and 14). Taken together these data demonstrate that subjects with upregulated lung IL-13 receive the greatest clinical benefits of IL-13 neutralising therapies such as tralokinumab.

### Example 2

### Measurement of IL-3 in Sputum

### Assay Description

Free IL-13 was measured using the Luminex technology (Millipore, Billerica, Mass.). Necessary reagents were supplied in the assay kit, all steps were conducted at room temperature, and the assay plate was sealed and placed on a plate shaker with moderate shaking for all incubation steps. Briefly, wells were pre-wet with 200 μl/well of Assay Buffer for 10 minutes. Anti-IL-13 antibody-conjugated bead was prepared in Bead Dilution Buffer and added to the assay plate at 25 μl/well; vacuum was applied to remove the Bead Dilution Buffer. Reference standards (RS), quality controls (QC), and negative control (NC) prepared in Assay Buffer were added to the assay plate at 25 μl/well. Test samples (neat serum) were added at 25 μl/well. The assay plate was incubated for 60±10 minutes. Unbound analyte was removed by washing the plate twice with 1× Wash Buffer. To detect bound analyte, biotin-conjugated anti-IL-13 detection antibody was added and the assay plate was incubated for 60±10 minutes. PE-conjugated Streptavidin dye (SA-PE) was then added at 25 μl/well and the assay plate was incubated for an additional 30±5 minutes. Excess detection antibody and dye was removed by washing the plate twice with 1× Wash Buffer. Luminex Sheath Fluid was added at 150 μl/well and the plate was placed on a plate shaker for ≧5 minutes prior to reading. The fluorescent intensity, measured in the Luminex200 Plate reader, was proportional to the IL-13 concentration in the sample. The presence of ≧1 pg/ml IL-13 in sputum from a subject with asthma was indicative that the subject had detectable IL-13 in sputum (“sputum IL-13 positive”). A value of <1 pg/ml IL-13 in sputum was indicative that the subject did not have detectable IL-13 in sputum and thus that the subject was “sputum IL-13-negative”.

### Reagents

1. MPXHCYTO-60K (Luminex Kit Cat# MPXHCYTO-60K):
   Filter Assay Plate: Cat# MX-PLATE
   Anti-IL-13 antibody-conjugated beads: Cat# MXHIL-13, bead region 50, 50× stock
   Reference Standards, rh IL-13: Cat# MXH8060
   Quality Control #1 & #2: MXH6060 & MXH6060-2
   Biotin-conjugated anti-IL-13 detection antibody: Cat# MXH 1060-1, 1× stock
   Streptavidin-Phycoerythrin detection dye: Cat# L-SAPE9, 1× stock
   Buffers: Assay Buffer (1×), Bead Dilution Buffer (1×), Wash Buffer (25×)
2. Luminex Sheath Fluid Cat#40-50000

### Assay Protocol

Assay reagents were equilibrated to room temperature. An aliquot of 200 μl of Assay Buffer was transferred to each well of a 96-well filter assay plate, the plate was sealed and incubated on a plate shaker for 10 minutes. Eight levels of reference standard (RS) were prepared by diluting the recombinant human IL-13 reference standards in Assay Buffer. The following concentrations were used: 2500.0, 833.3, 277.8, 92.6, 30.9, 10.3, 3.4 and 1.1 pg/ml. Three 3 levels of quality control samples (QC) were prepared in Assay Buffer. The following concentrations were used: 1000.0, 10.0 pg/ml. The negative control (NC) (0.0 ng/ml) was prepared with the Assay Buffer. The test serum samples were thawed on ice and vortexed briefly. The anti-IL-13 antibody-conjugated bead was diluted 1:50 in Bead Dilution buffer. A vacuum was applied to remove the buffer, the bottom of the plate was blotted on absorbent paper. An aliquot of 25 μl/well of the diluted bead solution was transferred to the plate. Vacuum was applied to the plate as above. Aliquots of 25 μl/well of the RS, QC, NC, and serum samples were transferred to the plate; which was then sealed and incubated for 60±10 minutes on a rotating platform with moderate shaking. 1× Wash Buffer was prepared from the 25× Wash Buffer stock. The plate was washed twice with 200 μl/well of 1× Wash Buffer. An aliquot of 25 μl/well of the biotin-conjugated anti-IL-13 detection antibody was transferred to the plate; the plate was sealed and incubated for 60±10 minutes on a rotating platform with moderate shaking. An aliquot of 25 μl/well of the Streptavidin dye (SA-PE) was transferred to the plate; the plate was sealed and incubated for 30±5 minutes on a rotating platform with moderate shaking. The plate was washed twice with 200 μl/well of 1× Wash Buffer. An aliquot of 150 μl/well of the Luminex Sheath Fluid was transferred to the plate and the plate was shaken on a plate shaker for ≧5 minutes. The plate was read on the Luminex200 Plate reader plate.

### Example 3

This is a Phase 2b, randomized, double-blind, placebo-controlled, parallel-arm, multicenter study to evaluate the efficacy and safety of two SC treatment regimens of tralokinumab in adult subjects with uncontrolled, severe asthma requiring high-dose ICS and LABA with or without additional controller medications (high-dose ICS defined as a total daily dose >500 μg fluticasone dry powder inhaler [DPI] or >440 μg metered dose inhaler [MDI]; GINA, 2009; National Heart, Lung, and Blood Institute, 2007). Subjects will be randomized in a 1:1 ratio to one of 2 cohorts (Cohort 1 or Cohort 2). Within each cohort, subjects will be randomized in a 2:1 ratio to receive tralokinumab (300 mg) or placebo as follows:

Cohort 1: Tralokinumab 300 mg (n=130) or Placebo (n=65) as 2 SC injections every 2 weeks (Q2W) for 50 weeks for a total of 26 doses
Cohort 2: Tralokinumab 300 mg (n=130) or Placebo (n=65) as 2 SC injections Q2W for 12 weeks followed by every 4 weeks (Q4W) for 38 weeks for a total of 16 doses

Subjects will be stratified at screening by the number of asthma exacerbations in the past 12 months (2 versus >2 but ≦6 exacerbations) and by chronic oral corticosteroid (OCS) use (presence versus absence).

A 5-week screening/run-in period (Week −5 to −1 [Day −1) will precede investigational product administration. Starting at Week −4 (Day −28), subjects will receive a fixed-dose combination product of fluticasone/salmeterol, either as an MDI (230 μg/21 μg) at a dose of 2 inhalations twice per day or as a DPI (500 μg/50 μg) at a dose of one inhalation twice per day. Sites/subjects will be permitted to use either presentation of fluticasone/salmeterol if approved and available for use in their country. If the subject is also taking additional asthma controller medications (including leukotriene modifiers, theophylline, cromones, or OCS≦20 mg/day), then these medications should be continued at a stable dose during the screening/run-in period. During the study, subjects may use inhaled reliever therapy (eg, short-acting β2 agonist or short-acting anticholinergic) on an as-required basis as documented in their Personalized Asthma Action Plan.

Subjects will continue to receive the same fixed-dose combination product of fluticasone/salmeterol with or without additional asthma controller medications at a stable dose during the treatment period and through Week 53. Subjects who experience an asthma exacerbation during the treatment period will be treated accordingly and will remain in the study. Subjects will return to the clinic at Week 53 for an assessment of the efficacy endpoints. Subjects will have 3 additional follow-up visits at Weeks 59, 67, and 75. After the Week 53 visit, background medications may be changed as deemed necessary by the investigator. As part of the study, selected sites will take part in a sub-study using HRCT scanning to measure potential airway wall structural changes including airway wall thickness. Sites will be selected on their ability and willingness to carry out the scans. Participation in this sub study is optional for subjects at the selected sites. An adequate number of sites will be identified in order to target recruitment of approximately 40 subjects into each of the 2 cohorts for the HRCT sub-study. Subjects will be in the study for 79 weeks, including a 5-week screening/run-in period (Week −5 to Week −1), a 48- or 50-week treatment period (Weeks 1-51 for Cohort 1 and Weeks 1-49 for Cohort 2), and a 24-week follow-up period (Weeks 51-75).

### Subject Population

The subjects in this study will be adults aged 18-75 years with uncontrolled, severe asthma requiring high-dose ICS and LABA with or without additional controller medications.

### Treatment Regimen

A total of 390 subjects will be randomized in a 1:1 ratio to one of 2 cohorts. Within each cohort, subjects will be randomized in a 2:1 ratio to receive SC tralokinumab (300 mg) or placebo. Investigational product (tralokinumab or placebo) will be administered as 2 SC injections of 1 mL either Q2W for 50 weeks for a total of 26 doses (Cohort 1) or Q2W for 12 weeks followed by Q4W for 38 weeks for a total of 16 doses (Cohort 2).

### REFERENCES

Bree A, Schlerman F J, Wadanoli M, Tchistiakova L, Marquette K, Tan X-Y, Jacobson B A, Widom A, Cook T A, Wood N, Vunnum S, Krykbaev R, Xu X, Donaldson D D, Goldman S J, Sypek J, Kasaian M T (2007) IL-13 blockade reduces lung inflammation after ascaris suum challenge in cynomolgus monkeys. J. Allergy Clin. Immunol. 119(5) 1251-7.
GINA Guidelines 2009: GINA report, Global strategy for asthma management and prevention. http://www.ginasthma.com/Guidelineitem.asp??11=2&12=1&intId=1561
Grunig G, Warnock M, Wakil A E, Venkayya R, Brombacher F, Rennick D M, Sheppard D, Mohrs M, Donaldson D D, Locksley R M, Corry D B. Requirement for IL-13 Independently of IL-4 in Experimental Asthma. Science 1998; 2261-3.
Heinzmann A, Mao X Q, Akaiwa M, Kreomer R T, Gao P S, Ohshima K, Umeshita R, Abe Y, Braun S, Yamashita T, Roberts M H, Sugimoto R, Arima K, Arinobu Y, Yu B, Kruse S, Enomoto T, Dake Y, Kawai M, Shimazu S, Sasaki S, Adra C N, Kitaichi M, Inoue H, Yamauchi K, Tomichi N, Kurimoto F, Hamasaki N, Hopkin J M, Izuhara K, Shirakawa T, Deichmann K A. Genetic variants of IL-13 signalling and human asthma and atopy. Hum Mol Genet. 2000; 9(4): 549-59.
Hershey G K. IL-13 receptors and signaling pathways: an evolving web. J Allergy Clin Immunol, 2003. 111(4): p. 677-90.
Howard T D, Whittaker P A, Zaiman A L, Koppelman G H, Xu J, Hanley M T, Meyers D A, Postma D S, Bleecker E R. Identification and association of polymorphisms in the interleukin-13 gene with asthma and atopy in a Dutch population. Am J Respir Cell Mol Biol. 2001; 25(3): 377-84.
Howard T D, Koppelman G H, Xu J, Zheng S L, Postma D S, Meyers D A, Bleecker E R. Gene-gene interaction in asthma: IL4RA and IL13 in a Dutch population with asthma. Am J Hum Genet. 2002; 70(1): 230-6.
Huang S-K, Xiao H-Q, Kleine-Tebbe J, Paciotti Gi, Marsh D G., Lichtenstein L M, and Liu M C. J. Immunol. 1995; 155(5): 2688-2694
Humbert M, Durham S R, Kimmitt P, Powell N, Assoufi B, Pfister R, Menz G, Kay A B, Corrigan C J. Elevated expression of messenger ribonucleic acid encoding IL-13 in the bronchial mucosa of atopic and nonatopic subjects with asthma. J Allergy Clin Immunol. 1997; 99(5): 657-65.
Juniper E F, O'Byrne P M, Guyatt G H, Ferrie P J, King D R. Development and validation of a questionnaire to measure asthma control. Eur Respir J. 1999; 14: 902-7.
Juniper E F, Bousquet J, Abetz L, Bateman E D. Identifying ‘well-controlled’ and ‘not well-controlled’ asthma using the Asthma Control Questionnaire. Respir Med. 2006; 100: 616-21.
Kauppi P, Lindblad-Toh K, Sevon P, Toivonen H T, Rioux J D, Villapakkam A, Laitinen L A, Hudson T J, Kere J, Laitinen T. A second-generation association study of the 5q31 cytokine gene cluster and the interleukin-4 receptor in asthma. Genomics. 2001 September; 77(1-2):35-42.
Komai-Koma M, McKay A, Thomson L, McSharry C, Chalmers G W, Liew F Y, Thomson N C. Immuno-regulatory cytokines in asthma: IL-15 and IL-13 in induced sputum. Clin Exp Allergy. 2001; 31(9): 1441-8.
Kotsimbos T C, Ernst P, Hamid Q A. Interleukin-13 and interleukin-4 are coexpressed in atopic asthma. Proc Assoc Am Physicians. 1996; 108(5): 368-73.
Kroegel, Julius, Matthys, Virchow, Luttmann. Endobronchial secretion of interleukin-13 following local allergen challenge in atopic asthma: relationship to interleukin-4 and eosinophil counts. Eur Respir J. 1996; 9: 899.
Liu X, Nickel R, Beyer K, Wahn U, Ehrlich E, Freidhoff L R, Bjorksten B, Beaty T H, Huang S K. An IL13 coding region variant is associated with a high total serum IgE level and atopic dermatitis in the German multicenter atopy study (MAS-90). J Allergy Clin Immunol. 2000; 106(1 Pt 1): 167-70.
Naseer T, Minshall E M, Leung D Y, Laberge S, Ernst P, Martin R J, Hamid Q. Expression of IL-12 and IL-13 mRNA in asthma and their modulation in response to steroid therapy. Am J Respir Crit Care Med. 1997; 155(3): 845-51.
Reddel H K, Taylor D R, Bateman E D, et at. An official American Thoracic Society/European Respiratory Society statement: asthma control and exacerbations: standardizing endpoints for clinical asthma trials and clinical practice. Am J Respir Crit Care Med 2009; 180: 59-99.
Saha S K, Berry M A, Parker D, Siddiqui S, Morgan A, May R, Monk P, Bradding P, Wardlaw A J, Pavord I D, Brightling C E. Increased sputum and bronchial biopsy IL-13 expression in severe asthma. J Allergy Clin Immunol. 2008 March; 121(3):685-91.
Tamura K, Suzuki M, Arakawa H, Tokuyama K, Morikawa A. Linkage and association studies of STATE gene polymorphisms and allergic diseases. Int Arch Allergy Immunol. 2003 May; 131(1):33-8.
van der Pouw Kraan T C, van Veen A, Boeije L C, van Tuyl S A, de Groot E R, Stapel S O, Bakker A, Verweij C L, Aarden L A, van der Zee J S. An IL-13 promoter polymorphism associated with increased risk of allergic asthma. Genes Immun. 1999; 1(1): 61-5.
Venkayya R, Lam M, Willkom M, Granig G, Corry D B, Erle D J. The Th2 lymphocyte products IL-4 and IL-13 rapidly induce airway hyperresponsiveness through direct effects on resident airway cells. Am J Respir Cell Mol Biol. 2002; 26(2): 202-8.
Vladich F D, Brazille S M, Stern D, Peck M L, Ghittoni R, Vercelli D. IL-13 R130Q, a common variant associated with allergy and asthma, enhances effector mechanisms essential for human allergic inflammation. J Clin Invest. 2005; 115(3): 747-54.
Wills-Karp M, Luyimbazi J, Xu X, Schofield B, Neben T Y, Karp C L, Donaldson D. Interleukin-13: Central mediator of allergic asthma Science 1998; 282: 2258-2261.
Zhu Z, Homer R J, Wang Z, Chen Q, Geba G P, Wang J, et al. Pulmonary expression of interleukin-13 causes inflammation, mucus hypersecretion, subepithelial fibrosis, physiologic abnormalities, and eotaxin production. J Clin Invest 1999; 103: 779-88.

### Sequence Listing Information

Tralokinumab (BAK502G9, CAT-354):
SEQ ID NO: 7 is HCDR1;
SEQ ID NO: 8 is HCDR2;
SEQ ID NO: 9 is HCDR3;
SEQ ID NO: 10 is LCDR1;
SEQ ID NO: 11 is LCDR2;
and
SEQ ID NO: 12 is LCDR3.
SEQ ID NO: 15 is VH fragment
SEQ ID NO: 16 is VL fragment
Variants of Tralokinumab (HCDR1, 2, 3, LCDR1, 2,
3)
BAK278D6 (SEQ ID NOS: 1, 2, 3, 4, 5, 6)
BAK1183H4 (SEQ ID NOS: 97, 98, 99, 100, 101, 102),
BAK1167F02 (SEQ ID NOS: 64, 65, 66, 67, 68, 69),
BAK1111D10 (SEQ ID NOS: 91, 92, 93, 94, 95, 96),
BAK1166G02 (SEQ ID NOS: 67, 68, 69, 70, 71, 72),
BAK1167F04 (SEQ ID NOS: 85, 86, 87, 88, 89, 90),
BAK1184C8 (SEQ ID NOS: 73, 74, 75, 76, 77, 78),
BAK1185E1 (SEQ ID NOS: 79, 80, 81, 82, 83, 84),
and
BAK1185F8(SEQ ID NOS: 103, 104, 105, 106, 107,
108).
Variants of tralokinumab (VH, VL)
BAK278D6 (VH SEQ ID NO: 13, VL SEQ ID NO; 14)
BAK1183H4 (VH SEQ ID NO: 37, VL SEQ ID NO: 38),
BAK1167F02 (VH SEQ ID NO: 35, VL SEQ ID NO: 36),
BAK1111D10 (VH SEQ ID NO: 41, VL SEQ ID NO: 42),
BAK1166G02 (VH SEQ ID NO: 53, VL SEQ ID NO: 54),
BAK1167F04 (VH SEQ ID NO: 43, VL SEQ ID NO: 44),
BAK1184C8 (VH SEQ ID NO: 45, VL SEQ ID NO: 46),
BAK1185E1 (VH SEQ ID NO: 47, VL SEQ ID NO: 48),
BAK1185F8 (VH SEQ ID NO: 49, VL SEQ ID NO: 50).
BAK278D6
HEAVY CHAIN
CDR1-SEQ ID NO 1: NYGLS
CDR2-SEQ ID NO 2: WISANNGDTNYGQEFQG
CDR3-SEQ ID NO 3: DSSSNWARWFFDL
BAK278D6
LIGHT CHAIN
CDR1-SEQ ID NO 4: GGNNIGSKLVH
CDR2-SEQ ID NO 5: DDGDRPS
CDR3-SEQ ID NO 6: QVWDTGSDPVV
BAK502G9
HEAVY CHAIN
CDR1-SEQ ID NO 7: NYGLS
CDR2-SEQ ID NO 8: WISANNGDTNYGQEFQG
CDR3-SEQ ID NO 9: DSSSSWARWFFDL
LIGHT CHAIN
CDR1-SEQ ID NO 10: GGNIIGSKLVH
CDR2-SEQ ID NO 11: DDGDRPS
CDR3-SEQ ID NO 12: QVWDTGSDPVV
BAK278D6
HEAVY CHAIN DOMAIN
SEQ ID NO 13:
EVQLVQSGAEVKKPGASVKVSCKASGYTFRNYGLSWVRQAPGQGLEWM
GWISANNGDTNYGQEFQGRITMTTETSTNTAHMELRSLRSDDTAVYYCV
RDSSSNWARWFFDLWGKGTMVTVSS
BAK278D6
LIGHT CHAIN DOMAIN
SEQ ID NO 14:
SYVLTQPPSVSVAPGQTARIPCGGNNIGSKLVHWYQQKPGQAPVLVVYD
DGDRPSGIPERFSGSNSGNTATLTISRIDAGDEADYYCQVWDTGSDPVVF
GGGTKLTVL
BAK502G9
HEAVY CHAIN DOMAIN
SEQ ID NO 15:
QVQLVQSGAEVKKPGASVKVSCKASGYTFTNYGLSWVRQAPGQGLEWM
GWISANNGDTNYGQEFQGRVTMTTDTSTSTAYMELRSLRSDDTAVYYCA
RDSSSSWARWFFDLWGRGTLVTVSS
BAK502G9
LIGHT CHAIN DOMAIN
SEQ ID NO 16:
SYVLTQPPSVSVAPGKTARITCGGNIIGSKLVHWYQQKPGQAPVLVIYDD
GDRPSGIPERFSGSNSGNTATLTISRVEAGDEADYYCQVWDTGSDPVVF
GGGTKLTVL
BAK278D6
HEAVY CHAIN
FR1-SEQ ID NO 17: EVQLVQSGAEVKKPGASVKVSCKASGYTFR
FR2-SEQ ID NO 18: WVRQAPGQGLEWMG
FR3-SEQ ID NO 19: RITMTTETSTNTAHMELRSLRSDDTAVYYCVR
BAK278D6
LIGHT CHAIN
FR1-SEQ ID NO 20: SYVLTQPPSVSVAPGQTARIPC
FR2-SEQ ID NO 21: WYQQKPGQAPVLVVY
FR3-SEQ ID NO 22: GIPERFSGSNSGNTATLTISRIDAGDEADYYC
BAK167A11
HEAVY CHAIN DOMAIN
SEQ ID NO 23:
EVQLLESGGGLVQPGGSLRLSCAASGFTFSSYAMSWVRQAPGKGLEWV
SAISGSGGSTYYADSVKGRFTISRDNSKNTLYLQMNSLRAEDTAVYYCAR
VGAAGEGYYGYWGRGTLVTVSS
BAK167A11
LIGHT CHAIN DOMAIN
SEQ ID NO 24:
NFMLTQPHSVSESPGKTVTISCTRSSGSIASNYVQWYQQRPGSAPTTVIY
DDNQRPSGVPDRFSGSIDSSSNSASLTISGLKTEDEADYYCQSYDSNND
VFGGGTKVTVL
BAK209B11
HEAVY CHAIN DOMAIN
SEQ ID NO 25:
QVQLQESGGGGLVQPGGSLRLSCAASGFTFSSYGMSWVRQAPGKGLE
WVSSISASGDSTFYADSVKGRFTISRDNNKNMVFLQVNSLRADDTAVYF
CAKDWSQWLVGDAFDVWGRGTTVTVSS
BAK209B11
LIGHT CHAIN DOMAIN
SEQ ID NO 26:
DIQLTQSPSTLSASVGDRVTITCRASQSVSLWVAWYQQRPGKAPKLLIYD
GSTLQSGVPARFSGSGSGTEFTLTISSLQPDDFATYYCQQYKTFSTFGQG
TKVEIKRA
BAK502G9
HEAVY CHAIN
FR1-SEQ ID NO 27: QVQLVQSGAEVKKPGASVKVSCKASGYTFT
FR2-SEQ ID NO 28: WYRQAPGQGLEWMG
FR3-SEQ ID NO 29: RVTMTTDTSTSTAYMELRSLRSDDTAVYYCAR
BAK502G9
LIGHT CHAIN
FR1-SEQ ID NO 30: SYVLTQPPSVSVAPGKTARITC
FR2-SEQ ID NO 31: WYQQKPGQAPVLVIY
FR3-SEQ ID NO 32: GIPERFSGSNSGNTATLTISRVEAGDEADYYC
BAK615E3
HEAVY CHAIN DOMAIN
SEQ ID NO 33:
EVQLLESGGGLVQPGGSLRLSCAASGFTFSSYAMSWVRQAPGKGLEWV
SAISGSGGSTYYADSVKGRFTISRDNSKNTLYLQMNSLRAEDTAVYYCAR
VGKATTEEGYYGYWGRGTLVTVSS
BAK615E3
LIGHT CHAIN DOMAIN
SEQ ID NO 34:
NFMLTQPHSVSESPGKTVTISCTRSSGSIASNYVQWYQQRPGSAPTTVIY
DDNQRPSGVPDRFSGSIDSSSNSASLTISGLKTEDEADYYCQSYDSNND
VFGGGTKVTVL
BAK1167F2
HEAVY CHAIN DOMAIN
SEQ ID NO 35:
QVQLVQSGAEVKKPGASVKVSCKASGYTFEQTGVSWVRQAPGQGLEW
MGWISANNGDTNYGQEFQGRVTMTTDTSTSTAYMELRSLRSDDTAVYY
CARDSSSSWARWFFDLWGRGTLVTVSS
BAK1167F2
LIGHT CHAIN DOMAIN
SEQ ID NO 36:
SYVLTQPPSVSVAPGKTARITCGGNIIGSKLVHWYQQKPGQAPVLVIYDD
GDRPSGIPERFSGSNSGNTATLTISRVEAGDEADYYCQVWDTGSDPVVF
GGGTKLTVL
BAK1183H4
HEAVY CHAIN DOMAIN
SEQ ID NO 37:
QVQLVQSGAEVKKPGASVKVSCKASGYTFTNYGLSWVRQAPGQGLEWM
GWINYDGGNTQYGQEFQGRVTMTTDTSTSTAYMELRSLRSDDTAVYYC
ARDSSSSWARWFFDLWGRGTLVTVSS
BAK1183H4
LIGHT CHAIN DOMAIN
SEQ ID NO 38:
SYVLTQPPSVSVAPGKTARITCGGNIIGSKLVHWYQQKPGQAPVLVIYDD
GDRPSGIPERFSGSNSGNTATLTISRVEAGDEADYYCQVWDTGSDPVVF
GGGTKLTVL
BAK1105H3
HEAVY CHAIN DOMAIN
SEQ ID NO 39:
QVQLVQSGAEVKKPGASVKVSCKASGYTFTNYGLSWVRQAPGQGLEWM
GWISGLNGETLYGQEFQGRVTMTTDTSTSTAYMELRSLRSDDTAVYYCA
RDSSSSWARWFFDLWGRGTLVTVSS
BAK1105H3
LIGHT CHAIN DOMAIN
SEQ ID NO 40:
SYVLTQPPSVSVAPGKTARITCGGNIIGSKLVHWYQQKPGQAPVLVIYDD
GDRPSGIPERFSGSNSGNTATLTISRVEAGDEADYYCQVWDTGSDPVVF
GGGTKLTVL
BAK1111D10
HEAVY CHAIN DOMAIN
SEQ ID NO 41:
QVQLVQSGAEVKKPGASVKVSCKASGYTFTNYGLSWVRQAPGQGLEWM
GWIATPDGQTSYGQEFQGRVTMTTDTSTSTAYMELRSLRSDDTAVYYCA
RDSNSSWARWFFDLWGRGTLVTVSS
BAK1111D10
LIGHT CHAIN DOMAIN
SEQ ID NO 42:
SYVLTQPPSVSVAPGKTARITCGGNIIGSKLVHWYQQKPGQAPVLVIYDD
GDRPSGIPERFSGSNSGNTATLTISRVEAGDEADYYCQVWDTGSDPVVF
GGGTKLTVL
BAK1167F4
HEAVY CHAIN DOMAIN
SEQ ID NO 43:
QVQLVQSGAEVKKPGASVKVSCKASGYTFIDTGVSWVRQAPGQGLEWM
GWISANNGDTNYGQEFQGRVTMTTDTSTSTAYMELRSLRSDDTAVYYCA
RDSSSSWARWFFDLWGRGTLVTVSS
BAK1167F4
LIGHT CHAIN DOMAIN
SEQ ID NO 44:
SYVLTQPPSVSVAPGKTARITCGGNIIGSKLVHWYQQKPGQAPVLVIYDD
GDRPSGIPERFSGSNSGNTATLTISRVEAGDEADYYCQVWDTGSDPVVF
GGGTKLTVL
BAK1184C8
HEAVY CHAIN DOMAIN
SEQ ID NO 45:
QVQLVQSGAEVKKPGASVKVSCKASGYTFTNYGLSWVRQAPGQGLEWM
GWISGSNGYTSYGQEFQGRVTMTTDTSTSTAYMELRSLRSDDTAVYYCA
RDSSSSWARWFFDLWGRGTLVTVSS
BAK1184C8
LIGHT CHAIN DOMAIN
SEQ ID NO 46:
SYVLTQPPSVSVAPGKTARITCGGNIIGSKLVHWYQQKPGQAPVLVIYDD
GDRPSGIPERFSGSNSGNTATLTISRVEAGDEADYYCQVWDTGSDPVVF
GGGTKLTVL
BAK1185E1
HEAVY CHAIN DOMAIN
SEQ ID NO 47:
QVQLVQSGAEVKKPGASVKVSCKASGYTFTNYGLSWVRQAPGQGLEWM
GWINDATGDTQYGQEFQGRVTMTTDTSTSTAYMELRSLRSDDTAVYYCA
RDSSSSWARWFFDLWGRGTLVTVSS
BAK1185E1
LIGHT CHAIN DOMAIN
SEQ ID NO 48:
SYVLTQPPSVSVAPGKTARITCGGNIIGSKLVHWYQQKPGQAPVLVIYDD
GDRPSGIPERFSGSNSGNTATLTISRVEAGDEADYYCQVWDTGSDPVVF
GGGTKLTVL
BAK1185F8
HEAVY CHAIN DOMAIN
SEQ ID NO 49:
QVQLVQSGAEVKKPGASVKVSCKASGYTFTDYGLSWVRQAPGQGLEW
MGWIRNIDGYTIYGQEFQGRVTMTTDTSTSTAYMELRSLRSDDTAVYYCA
RDSSSSWARWFFDLWGRGTLVTVSS
BAK1185F8
LIGHT CHAIN DOMAIN
SEQ ID NO 50:
SYVLTQPPSVSVAPGKTARITCGGNIIGSKLVHWYQQKPGQAPVLVIYDD
GDRPSGIPERFSGSNSGNTATLTISRVEAGDEADYYCQVWDTGSDPVVF
GGGTKLTVL
BAK1187B4
HEAVY CHAIN DOMAIN
SEQ ID NO 51:
QVQLVQSGAEVKKPGASVKVSCKASGYTFTNYGLSWVRQAPGQGLEWM
GWIDDDSGTTIYGQEFQGRVTMTTDTSTSTAYMELRSLRSDDTAVYYCA
RDSSSSWARWFFDLWGRGTLVTVSS
BAK1187B4
LIGHT CHAIN DOMAIN
SEQ ID NO 52:
SYVLTQPPSVSVAPGKTARITCGGNIIGSKLVHWYQQKPGQAPVLVIYDD
GDRPSGIPERFSGSNSGNTATLTISRVEAGDEADYYCQVWDTGSDPVVF
GGGTKLTVL
BAK1166G2
HEAVY CHAIN DOMAIN
SEQ ID NO 53:
QVQLVQSGAEVKKPGASVKVSCKASGYTFANTGISWVRQAPGQGLEWM
GWISANNGDTNYGQEFQGRVTMTTDTSTSTAYMELRSLRSDDTAVYYCA
RDSSSSWARWFFDLWGRGTLVTVSS
BAK1166G2
LIGHT CHAIN DOMAIN
SEQ ID NO 54:
SYVLTQPPSVSVAPGKTARITCGGNIIGSKLVHWYQQKPGQAPVLVIYDD
GDRPSGIPERFSGSNSGNTATLTISRVEAGDEADYYCQVWDTGSDPVVF
GGGTKLTVL
BAK167A11
HEAVY CHAIN
CDR1-SEQ ID NO 55: SYAMS
CDR2-SEQ ID NO 56: AISGSGGSTYYADSVKG
CDR3-SEQ ID NO 57: VGAAGEGYYGY
BAK16A11
LIGHT CHAIN
CDR1-SEQ ID NO 58: TRSSGSIASNYVQ
CDR2-SEQ ID NO 59: DDNQRPS
CDR3-SEQ ID NO 60: QSYDSNNDV
BAK1167F2
HEAVY CHAIN
CDR1-SEQ ID NO 61: QTGVS
CDR2-SEQ ID NO 62: WISANNGDTNYGQEFQG
CDR3-SEQ ID NO 63: DSSSSWARWFFDL
BAK1167F2
LIGHT CHAIN
CDR1-SEQ ID NO 64: GGNIIGSKLVH
CDR2-SEQ ID NO 65: DDGDRPS
CDR3-SEQ ID NO 66: QVWDTGSDPVV
BAK1166G2
HEAVY CHAIN
CDR1-SEQ ID NO 67: NTGIS
CDR2-SEQ ID NO 68: WISANNGDTNYGQEFQG
CDR3-SEQ ID NO 69: DSSSSWARWFFDL
BAK1166G2
LIGHT CHAIN
CDR1-SEQ ID NO 70: GGNIIGSKLVH
CDR2-SEQ ID NO 71: DDGDRPS
CDR3-SEQ ID NO 72: QVWDTGSDPVV
BAK1184C8
HEAVY CHAIN
CDR1-SEQ ID NO 73: NYGLS
CDR2-SEQ ID NO 74: WISGSNGYTSYGQEFQG
CDR3-SEQ ID NO 75: DSSSSWARWFFDL
BAK1184C8
LIGHT CHAIN
CDR1-SEQ ID NO 76: GGNIIGSKLVH
CDR2-SEQ ID NO 77: DDGDRPS
CDR3-SEQ ID NO 78: QVWDTGSDPVV
BAK1185E1
HEAVY CHAIN
CDR1-SEQ ID NO 79: NYGLS
CDR2-SEQ ID NO 80: WINDATGDTQYGQEFQG
CDR3-SEQ ID NO 81: DSSSSWARWFFDL
BAK1185E1
LIGHT CHAIN
CDR1-SEQ ID NO 82: GGNIIGSKLVH
CDR2-SEQ ID NO 83: DDGDRPS
CDR3-SEQ ID NO 84: QVWDTGSDPVV
BAK1167F4
HEAVY CHAIN
CDR1-SEQ ID NO 85: DTGVS
CDR2-SEQ ID NO 86: WISANNGDTNYGQEFQG
CDR3-SEQ ID NO 87: DSSSSWARWFFDL
BAK1167F4
LIGHT CHAIN
CDR1-SEQ ID NO 88: GGNIIGSKLVH
CDR2-SEQ ID NO 89: DDGDRPS
CDR3-SEQ ID NO 90: QVWDTGSDPVV
BAK1111D10
HEAVY CHAIN
CDR1-SEQ ID NO 91: NYGLS
CDR2-SEQ ID NO 92: WIATPDGQTSYGQEFQG
CDR3-SEQ ID NO 93: DSNSSWARWFFDL
BAK1111D10
LIGHT CHAIN
CDR1-SEQ ID NO 94: GGNIIGSKLVH
CDR2-SEQ ID NO 95: DDGDRPS
CDR3-SEQ ID NO 96: QVWDTGSDPVV
BAK1183H4
HEAVY CHAIN
CDR1-SEQ ID NO 97: NYGLS
CDR2-SEQ ID NO 98: WINYDGGNTQYGQEFQG
CDR3-SEQ ID NO 99: DSSSSWARWFFDL
BAK1183H4
LIGHT CHAIN
CDR1-SEQ ID NO 100: GGNIIGSKLVH
CDR2-SEQ ID NO 101: DDGDRPS
CDR3-SEQ ID NO 102: QVWDTGSDPVV
BAK1185H8
HEAVY CHAIN
CDR1-SEQ ID NO 103: DYGLS
CDR2-SEQ ID NO 104: WRINDGYTIYGQEFQG
CDR3-SEQ ID NO 105: DSSSSWARWFFDL
BAK1185H8
LIGHT CHAIN
CDR1-SEQ ID NO 106: GGNIIGSKLVH
CDR2-SEQ ID NO 107: DDGDRPS
CDR3-SEQ ID NO 108: QVWDTGSDPVV
BAK278D6
HEAVY CHAIN-SEQ ID NO: 109
CAGGTGCAGCTGGTGCAGTCTGGGGCTGAGGTGAAGAAGCCTGGGG
CCTCAGTGAAGGTCTCCTGCAAGGCTTCTGGTTACACCTTTACAAATTA
TGGTCTCAGCTGGGTGCGACAGGCCCCTGGACAAGGGCTTGAGTGGA
TGGGATGGATCAGCGCTAATAATGGCGACACAAATTATGGACAGGAAT
TCCAGGGCAGAGTCACCATGACCACAGATACATCCACGAGCACAGCCT
ACATGGAGTTGAGGAGCCTGAGATCTGACGACACGGCCGTTTATTACT
GTGCGAGAGACTCCAGCAGCAACTGGGCCCGCTGGTTTTTCGATCTCT
GGGGCCGGGGGACACTGGTCACCGTCTCCTCA
BAK278D6
LIGHT CHAIN-SEQ ID NO: 110
TCCTATGTGCTGACTCAGCCACCCTCGGTGTCAGTGGCCCCAGGTAAG
ACGGCCAGGATTACCTGTGGGGGAAACAACATTGGAAGTAAACTTGTA
CACTGGTACCAGCAGAAGCCAGGCCAGGCCCCTGTGCTGGTCATCTAT
GATGATGGCGACCGGCCCTCAGGGATCCCTGAGCGATTCTCTGGCTC
CAACTCTGGGAACACGGCCACCCTGACCATCAGCAGGGTCGAGGCCG
GGGATGAGGCCGACTATTATTGTCAGGTGTGGGATACTGGTAGTGATC
CCGTGGTATTCGGCGGAGGGACCAAGCTGACCGTCCTAGGT
BAK502G9
HEAVY CHAIN-SEQ ID NO: 111
CAGGTCCAGCTGGTGCAGTCTGGGGCTGAGGTGAAGAAGCCTGGGGC
CTCAGTGAAGGTCTCCTGCAAGGCTTCTGGTTACACCTTTACAAATTAT
GGTCTCAGCTGGGTGCGACAGGCCCCTGGACAAGGGCTTGAGTGGAT
GGGATGGATCAGCGCTAATAATGGCGACACAAATTATGGACAGGAATT
CCAGGGCAGAGTCACCATGACCACAGATACATCCACGAGCACAGCCTA
CATGGAGTTGAGGAGCCTGAGATCTGACGACACGGCCGTTTATTACTG
TGCGAGAGACTCCAGCAGCAGCTGGGCCCGCTGGTTTTTCGATCTCTG
GGGCCGGGGGACACTGGTCACCGTCTCCTCA
BAK502G9
LIGHT CHAIN-SEQ ID NO: 112
TCCTATGTGCTGACTCAGCCACCCTCGGTGTCAGTGGCCCCAGGAAAG
ACGGCCAGGATTACCTGTGGGGGAAACATCATTGGAAGTAAACTTGTA
CACTGGTACCAGCAGAAGCCAGGCCAGGCCCCTGTGCTGGTCATCTAT
GATGATGGCGACCGGCCCTCAGGGATCCCTGAGCGATTCTCTGGCTC
CAACTCTGGGAACACGGCCACCCTGACCATCAGCAGGGTCGAGGCCG
GGGATGAGGCCGACTATTATTGTCAGGTGTGGGATACTGGTAGTGATC
CCGTGGTATTCGGCGGAGGGACCAAGCTGACCGTCCTAGGT
BAK1105H03
HEAVY CHAIN-SEQ ID NO: 113
CAGGTCCAGCTGGTGCAGTCTGGGGCTGAGGTGAAGAAGCCTGGGGC
CTCAGTGAAGGTCTCCTGCAAGGCTTCTGGTTACACCTTTACAAATTAT
GGTCTCAGCTGGGTGCGACAGGCCCCTGGACAAGGGCTTGAGTGGAT
GGGATGGATCTCCGGCTTGAACGGCGAGACATTGTATGGACAGGAATT
CCAGGGCAGAGTCACCATGACCACAGATACATCCACGAGCACAGCCTA
CATGGAGTTGAGGAGCCTGAGATCTGACGACACGGCCGTTTATTACTG
TGCGAGAGACTCCAGCAGCAGCTGGGCCCGCTGGTTTTTCGATCTCTG
GGGCCGGGGGACACTGGTCACCGTCTCCTCA
BAK1105H03
LIGHT CHAIN-SEQ ID NO: 114
TCCTATGTGCTGACTCAGCCACCCTCGGTGTCAGTGGCCCCAGGAAAG
ACGGCCAGGATTACCTGTGGGGGAAACATCATTGGAAGTAAACTTGTA
CACTGGTACCAGCAGAAGCCAGGCCAGGCCCCTGTGCTGGTCATCTAT
GATGATGGCGACCGGCCCTCAGGGATCCCTGAGCGATTCTCTGGCTC
CAACTCTGGGAACACGGCCACCCTGACCATCAGCAGGGTCGAGGCCG
GGGATGAGGCCGACTATTATTGTCAGGTGTGGGATACTGGTAGTGATC
CCGTGGTATTCGGCGGAGGGACCAAGCTGACCGTCCTAGGT
BAK1111D10
HEAVY CHAIN-SEQ ID NO: 115
CAGGTGCAGCTGGTGCAGTCTGGGGCTGAGGTGAAGAAGCCTGGGG
CCTCAGTGAAGGTCTCCTGCAAGGCTTCTGGTTACACCTTTACAAATTA
TGGTCTCAGCTGGGTGCGACAGGCCCCTGGACAAGGGCTTGAGTGGA
TGGGATGGATCGCAACCCCAGACGGCCAGACAAGCTATGGACAGGAA
TTCCAGGGCAGAGTCACCATGACCACAGATACATCCACGAGCACAGCC
TACATGGAGTTGAGGAGCCTGAGATCTGACGACACGGCCGTTTATTAC
TGTGCGAGAGACTCCAACAGCAGCTGGGCCCGCTGGTTTTTCGATCTC
TGGGGCCGGGGGACACTGGTCACCGTCTCCTCA
BAK1111D10
LIGHT CHAIN-SEQ ID NO: 116
TCCTATGTGCTGACTCAGCCACCCTCGGTGTCAGTGGCCCCAGGAAAG
ACGGCCAGGATTACCTGTGGGGGAAACATCATTGGAAGTAAACTTGTA
CACTGGTACCAGCAGAAGCCAGGCCAGGCCCCTGTGCTGGTCATCTAT
GATGATGGCGACCGGCCCTCAGGGATCCCTGAGCGATTCTCTGGCTC
CAACTCTGGGAACACGGCCACCCTGACCATCAGCAGGGTCGAGGCCG
GGGATGAGGCCGACTATTATTGTCAGGTGTGGGATACTGGTAGTGATC
CCGTGGTATTCGGCGGAGGGACCAAGCTGACCGTCCTAGGT
BAK 1167F2
HEAVY CHAIN-SEQ ID NO: 117
CAAGTGCAGCTGGTGCAGTCTGGGGCTGAGGTGAAGAAGCCTGGGGC
CTCAGTGAAGGTCTCCTGCAAGGCTTCTGGTTACACCTTTGAGCAGAC
CGGCGTCTCCTGGGTGCGACAGGCCCCTGGACAAGGGCTTGAGTGGA
TGGGATGGATCAGCGCTAATAATGGCGACACAAATTATGGACAGGAAT
TCCAGGGCAGAGTCACCATGACCACAGATACATCCACGAGCACAGCCT
ACATGGAGTTGAGGAGCCTGAGATCTGACGACACGGCCGTTTATTACT
GTGCGAGAGACTCCAGCAGCAGCTGGGCCCGCTGGTTTTTCGATCTCT
GGGGCCGGGGGACACTGGTCACCGTCTCCTCA
BAK 1167F2
LIGHT CHAIN-SEQ ID NO: 118
TCCTATGTGCTGACTCAGCCACCCTCGGTGTCAGTGGCCCCAGGAAAG
ACGGCCAGGATTACCTGTGGGGGAAACATCATTGGAAGTAAACTTGTA
CACTGGTACCAGCAGAAGCCAGGCCAGGCCCCTGTGCTGGTCATCTAT
GATGATGGCGACCGGCCCTCAGGGATCCCTGAGCGATTCTCTGGCTC
CAACTCTGGGAACACGGCCACCCTGACCATCAGCAGGGTCGAGGCCG
GGGATGAGGCCGACTATTATTGTCAGGTGTGGGATACTGGTAGTGATC
CCGTGGTATTCGGCGGAGGGACCAAGCTGACCGTCCTAGGT
BAK 1167F04
HEAVY CHAIN-SEQ ID NO: 119
CAGGTGCAGCTGGTGCAGTCTGGGGCTGAGGTGAAGAAGCCTGGGG
CCTCAGTGAAGGTCTCCTGCAAGGCTTCTGGTTACACCTTTATCGACAC
CGGGGTCTCCTGGGTGCGACAGGCCCCTGGACAAGGGCTTGAGTGGA
TGGGATGGATCAGCGCTAATAATGGCGACACAAATTATGGACAGGAAT
TCCAGGGCAGAGTCACCATGACCACAGATACATCCACGAGCACAGCCT
ACATGGAGTTGAGGAGCCTGAGATCTGACGACACGGCCGTTTATTACT
GTGCGAGAGACTCCAGCAGCAGCTGGGCCCGCTGGTTTTTCGATCTCT
GGGGCCGGGGGACACTGGTCACCGTCTCCTCA
BAK 1167F04
LIGHT CHAIN-SEQ ID NO: 120
TCCTATGTGCTGACTCAGCCACCCTCGGTGTCAGTGGCCCCAGGAAAG
ACGGCCAGGATTACCTGTGGGGGAAACATCATTGGAAGTAAACTTGTA
CACTGGTACCAGCAGAAGCCAGGCCAGGCCCCTGTGCTGGTCATCTAT
GATGATGGCGACCGGCCCTCAGGGATCCCTGAGCGATTCTCTGGCTC
CAACTCTGGGAACACGGCCACCCTGACCATCAGCAGGGTCGAGGCCG
GGGATGAGGCCGACTATTATTGTCAGGTGTGGGATACTGGTAGTGATC
CCGTGGTATTCGGCGGAGGGACCAAGCTGACCGTCCTAGGT
BAK 1183H4
HEAVY CHAIN-SEQ ID NO: 121
CAGGTGCAGCTGGTGCAGTCTGGGGCTGAGGTGAAGAAGCCTGGGG
CCTCAGTGAAGGTCTCCTGCAAGGCTTCTGGTTACACCTTTACAAATTA
TGGTCTCAGCTGGGTGCGACAGGCCCCTGGACAAGGGCTTGAGTGGA
TGGGATGGATCAACTACGACGGCGGCAACACACAGTATGGACAGGAAT
TCCAGGGCAGAGTCACCATGACCACAGATACATCCACGAGCACAGCCT
ACATGGAGTTGAGGAGCCTGAGATCTGACGACACGGCCGTTTATTACT
GTGCGAGAGACTCCAGCAGCAGCTGGGCCCGCTGGTTTTTCGATCTCT
GGGGCCGGGGGACACTGGTCACCGTCTCCTCA
BAK 1183H4
LIGHT CHAIN-SEQ ID NO: 122
TCCTATGTGCTGACTCAGCCACCCTCGGTGTCAGTGGCCCCAGGAAAG
ACGGCCAGGATTACCTGTGGGGGAAACATCATTGGAAGTAAACTTGTA
CACTGGTACCAGCAGAAGCCAGGCCAGGCCCCTGTGCTGGTCATCTAT
GATGATGGCGACCGGCCCTCAGGGATCCCTGAGCGATTCTCTGGCTC
CAACTCTGGGAACACGGCCACCCTGACCATCAGCAGGGTCGAGGCCG
GGGATGAGGCCGACTATTATTGTCAGGTGTGGGATACTGGTAGTGATC
CCGTGGTATTCGGCGGAGGGACCAAGCTGACCGTCCTAGGT
BAK1184C8
HEAVY CHAIN-SEQ ID NO: 123
CAGGTGCAGCTGGTGCAGTCTGGGGCTGAGGTGAAGAAGCCTGGGG
CCTCAGTGAAGGTCTCCTGCAAGGCTTCTGGTTACACCTTTACAAATTA
TGGTCTCAGCTGGGTGCGACAGGCCCCTGGACAAGGGCTTGAGTGGA
TGGGATGGATCAGCGGGAGCAACGGCTACACATCTTATGGACAGGAAT
TCCAGGGCAGAGTCACCATGACCACAGATACGTCCACGAGCACAGCCT
ACATGGAGTTGAGGAGCCTGAGATCTGACGACACGGCCGTTTATTACT
GTGCGAGAGACTCCAGCAGCAGCTGGGCCCGCTGGTTTTTCGATCTCT
GGGGCCGGGGGACACTGGTCACCGTCTCCTCA
BAK1184C8
LIGHT CHAIN-SEQ ID NO: 124
TCCTATGTGCTGACTCAGCCACCCTCGGTGTCAGTGGCCCCAGGAAAG
ACGGCCAGGATTACCTGTGGGGGAAACATCATTGGAAGTAAACTTGTA
CACTGGTACCAGCAGAAGCCAGGCCAGGCCCCTGTGCTGGTCATCTAT
GATGATGGCGACCGGCCCTCAGGGATCCCTGAGCGATTCTCTGGCTC
CAACTCTGGGAACACGGCCACCCTGACCATCAGCAGGGTCGAGGCCG
GGGATGAGGCCGACTATTATTGTCAGGTGTGGGATACTGGTAGTGATC
CCGTGGTATTCGGCGGAGGGACCAAGCTGACCGTCCTAGGT
BAK1185E1
HEAVY CHAIN-SEQ ID NO: 125
CAGGTGCAGCTGGTGCAGTCTGGGGCTGAGGTGAAGAAGCCTGGGG
CCTCAGTGAAGGTCTCCTGCAAGGCTTCTGGTTACACCTTTACAAATTA
TGGTCTCAGCTGGGTGCGACAGGCCCCTGGACAAGGGCTTGAGTGGA
TGGGATGGATCAACGACGCCACCGGCGACACACAGTATGGACAGGAA
TTCCAGGGCAGAGTCACCATGACCACAGATACATCCACGAGCACAGCC
TACATGGAGTTGAGGAGCCTGAGATCTGACGACACGGCCGTTTATTAC
TGTGCGAGAGACTCCAGCAGCAGCTGGGCCCGCTGGTTTTTCGATCTC
TGGGGCCGGGGGACACTGGTCACCGTCTCCTCA
BAK1185E1
LIGHT CHAIN-SEQ ID NO: 126
TCCTATGTGCTGACTCAGCCACCCTCGGTGTCAGTGGCCCCAGGAAAG
ACGGCCAGGATTACCTGTGGGGGAAACATCATTGGAAGTAAACTTGTA
CACTGGTACCAGCAGAAGCCAGGCCAGGCCCCTGTGCTGGTCATCTAT
GATGATGGCGACCGGCCCTCAGGGATCCCTGAGCGATTCTCTGGCTC
CAACTCTGGGAACACGGCCACCCTGACCATCAGCAGGGTCGAGGCCG
GGGATGAGGCCGACTATTATTGTCAGGTGTGGGATACTGGTAGTGATC
CCGTGGTATTCGGCGGAGGGACCAAGCTGACCGTCCTAGGT
BAK1185F8
HEAVY CHAIN-SEQ ID NO: 127
CAGGTGCAGCTGGTGCAGTCTGGGGCTGAGGTGAAGAAGCCTGGGG
CCTCAGTGAAGGTCTCCTGCAAGGCTTCTGGTTACACCTTTACAGATTA
TGGTCTCAGCTGGGTGCGACAGGCCCCTGGACAAGGGCTAGAGTGGA
TGGGATGGATCCGCAACATCGACGGCTACACAATTTATGGACAGGAAT
TCCAGGGCAGAGTCACCATGACCACAGATACATCCACGAGCACAGCCT
ACATGGAGTTGAGGAGCCTGAGATCTGACGACACGGCCGTTTATTACT
GTGCGAGAGACTCCAGCAGCAGCTGGGCCCGCTGGTTTTTCGATCTCT
GGGGCCGGGGGACACTGGTCACCGTCTCCTCA
BAK1185F8
LIGHT CHAIN-SEQ ID NO: 128
TCCTATGTGCTGACTCAGCCACCCTCGGTGTCAGTGGCCCCAGGAAAG
ACGGCCAGGATTACCTGTGGGGGAAACATCATTGGAAGTAAACTTGTA
CACTGGTACCAGCAGAAGCCAGGCCAGGCCCCTGTGCTGGTCATCTAT
GATGATGGCGACCGGCCCTCAGGGATCCCTGAGCGATTCTCTGGCTC
CAACTCTGGGAACACGGCCACCCTGACCATCAGCAGGGTCGAGGCCG
GGGATGAGGCCGACTATTATTGTCAGGTGTGGGATACTGGTAGTGATC
CCGTGGTATTCGGCGGAGGGACCAAGCTGACCGTCCTAGGT
BAK1187B4
HEAVY CHAIN-SEQ ID NO: 129
CAGGTCCAGCTGGTGCAGTCTGGGGCTGAGGTGAAGAAGCCTGGGGC
CTCAGTGAAGGTCTCCTGCAAGGCTTCTGGTTACACCTTTACAAATTAT
GGTCTCAGCTGGGTGCGACAGGCCCCTGGACAAGGGCTTGAGTGGAT
GGGATGGATCGACGACGACAGCGGCACGACAATATATGGACAGGAAT
TCCAGGGCAGAGTCACCATGACCACAGATACATCCACGAGCACAGCCT
ACATGGAGTTGAGGAGCCTGAGATCTGACGACACGGCCGTTTATTACT
GTGCGAGAGACTCCAGCAGCAGCTGGGCCCGCTGGTTTTTCGATCTCT
GGGGCCGGGGGACACTGGTCACCGTCTCCTCA
BAK1187B4
LIGHT CHAIN-SEQ ID NO: 130
TCCTATGTGCTGACTCAGCCACCCTCGGTGTCAGTGGCCCCAGGAAAG
ACGGCCAGGATTACCTGTGGGGGAAACATCATTGGAAGTAAACTTGTA
CACTGGTACCAGCAGAAGCCAGGCCAGGCCCCTGTGCTGGTCATCTAT
GATGATGGCGACCGGCCCTCAGGGATCCCTGAGCGATTCTCTGGCTC
CAACTCTGGGAACACGGCCACCCTGACCATCAGCAGGGTCGAGGCCG
GGGATGAGGCCGACTATTATTGTCAGGTGTGGGATACTGGTAGTGATC
CCGTGGTATTCGGCGGAGGGACCAAGCTGACCGTCCTAGGT
BAK1166G02
HEAVY CHAIN-SEQ ID NO: 131
CAAGTGCAGTTGGTGCAGTCTGGGGCTGAGGTGAAGAAGCCTGGGGC
CTCAGTGAAGGTCTCCTGCAAGGCTTCTGGTTACACCTTTGCGAACAC
CGGGATCTCGTGGGTGCGACAGGCCCCTGGACAAGGGCTTGAGTGGA
TGGGATGGATCAGCGCTAATAATGGCGACACAAATTATGGACAGGAAT
TCCAGGGCAGAGTCACCATGACCACAGATACATCCACGAGCACAGCCT
ACATGGAGTTGAGGAGCCTGAGATCTGACGACACGGCCGTTTATTACT
GTGCGAGAGACTCCAGCAGCAGCTGGGCCCGCTGGTTTTTCGATCTCT
GGGGTCGGGGGACACTGGTCACCGTCTCCTCA
BAK1166G02
LIGHT CHAIN-SEQ ID NO: 132
TCCTATGTGCTGACTCAGCCACCCTCGGTGTCAGTGGCCCCAGGAAAG
ACGGCCAGGATTACCTGTGGGGGAAACATCATTGGAAGTAAACTTGTA
CACTGGTACCAGCAGAAGCCAGGCCAGGCCCCTGTGCTGGTCATCTAT
GATGATGGCGACCGGCCCTCAGGGATCCCTGAGCGATTCTCTGGCTC
CAACTCTGGGAACACGGCCACCCTGACCATCAGCAGGGTCGAGGCCG
GGGATGAGGCCGACTATTATTGTCAGGTGTGGGATACTGGTAGTGATC
CCGTGGTATTCGGCGGAGGGACCAAGCTGACCGTCCTAGGT
BAK165E7
HEAVY CHAIN-SEQ ID NO: 133
EVQLVQSGAEVKKPGASVKVSCKASGYTFTNYGLSWVRQAPGQGLEWM
GWISANNGETNYGQEFQGRVTMTTETPTNTAHMELRSLTSDDTAVYYCV
RDSSSNWARWYFDLWGQGTLVTVSS
BAK165E7
LIGHT CHAIN-SEQ ID NO: 134
SYVLTQPPSVSVAPGQTARIPCGGNNIGSKLVHWYQQKPGQAPVLVVYD
DGDRPSGIPERFSGSNSGNTATLTISRIDAGDEADYYCQVWDTGSDPVVF
GGGTKLTVLG
BAK165E7
HEAVY CHAIN
CDR1-SEQ ID NO: 135 NYGLS
CDR2-SEQ ID NO: 136 WISANNGETNYGQEFQG
CDR3-SEQ ID NO: 137 DSSSNWARWYFDL
BAK165E7
LIGHT CHAIN
CDR1-SEQ ID NO: 138 GGNNIGSKLVH
CDR2-SEQ ID NO: 139 DDGDRPS
CDR3-SEQ ID NO: 140 QVWDTGSDPVV
BAK582F7
HEAVY CHAIN
CDR1-SEQ ID NO 141: SYAMS
CDR2-SEQ ID NO 142: AISGSGGSTYYADSVKG
CDR3-SEQ ID NO 143: VGAAGEGYYGY
BAK582F7
LIGHT CHAIN
CDR1-SEQ ID NO 144: TRSSGSIASNYVE
CDR2-SEQ ID NO 145: DDNQRPS
CDR3-SEQ ID NO 146: QSYDSNNDV
BAK612B5
HEAVY CHAIN
CDR1-SEQ ID NO 147: SYAMS
CDR2-SEQ ID NO 148: AISGSGGSTYYADSVKG
CDR3-SEQ ID NO 149: VGRATTDEGYYGY
BAK612B5
LIGHT CHAIN
CDR1-SEQ ID NO 150: TRSSGSIASNYVQ
CDR2-SEQ ID NO 151: DDNQRPS
CDR3-SEQ ID NO 152: QSYDSNNDV
BAK615E3
HEAVY CHAIN
CDR1-SEQ ID NO 153: SYAMS
CDR2-SEQ ID NO 154: AISGSGGSTYYADSVKG
CDR3-SEQ ID NO 155: VGKATTEEGYY
BAK615E3
LIGHT CHAIN
CDR1-SEQ ID NO 156: TRSSGSIASNYVQ
CDR2-SEQ ID NO 157: DDNQRPS
CDR3-SEQ ID NO 158: QSYDSNNDV
BAK0278D6
HEAVY CHAIN
CDR1-SEQ ID NO 159: AATTATGGTCTCAGC
CDR2-SEQ ID NO 160:
TGGATCAGCGCTAATAATGGCGACACAAATTATGGACAGGAATTCCAG
GGC
CDR3-SEQ ID NO 161:
GACTCCAGCAGCAACTGGGCCCGCTGGTTTTTCGATCTC
BAK278D6
LIGHT CHAIN
CDR1-SEQ ID NO 162:
GGGGGAAACAACATTGGAAGTAAACTTGTACAC
CDR2-SEQ ID NO 163: GATGATGGCGACCGGCCCTCA
CDR3-SEQ ID NO 164:
CAGGTGTGGGATACTGGTAGTGATCCCGTGGTA
BAK502G9
HEAVY CHAIN
CDR1-SEQ ID NO 165: AATTATGGTCTCAGC
CDR2-SEQ ID NO 166:
TGGATCAGCGCTAATAATGGCGACACAAATTATGGACAGGAATTCCAG
GGC
CDR3-SEQ ID NO 167:
GACTCCAGCAGCAGCTGGGCCCGCTGGTTTTTCGATCTC
BAK502G9
LIGHT CHAIN
CDR1-SEQ ID NO 168:
GGGGGAAACATCATTGGAAGTAAACTTGTACAC
CDR2-SEQ ID NO 169: GATGATGGCGACCGGCCCTCA
CDR3-SEQ ID NO 170:
CAGGTGTGGGATACTGGTAGTGATCCCGTGGTA
CH Domains-SEQ ID NO: 171
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPSCPAPEFLGGPSVFLFPPKPKDTLMISRTPEVTCVVVDVSQED
PEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCL
VKGFYPSDIAVEWESNGQPENNYKTTPPVLDSDGSFFLYSRLTVDKSRW
QEGNVFSCSVMHEALHNHYTQKSLSLSLGK
CL Domain-SEQ ID NO: 172
QPKAAPSVTLFPPSSEELQANKATLVCLISDFYPGAVTVAWKADSSPVKA
GVETTTPSKQSNNKYAASSYLSLTPEQWKSHRSYSCQVTHEGSTVEKTV
APTECS

1. (canceled)

2. A method of treating a subject with impaired pulmonary function, said method comprising administering to said subject an IL-13 antagonist in an amount sufficient to improve, maintain or reduce the rate of decline of pulmonary function.

3. (canceled)

4. A method of improving, maintaining or reducing the rate of decline in pulmonary function in a subject with impaired pulmonary function, said method comprising administering to said subject an IL-13 antagonist.

5-8. (canceled)

9. The method of claim 1, wherein pulmonary function is assessed by a spirometry method selected from measuring FEV1, PEF and FVC.

10. (canceled)

11. The method of claim 1, wherein the IL-13 antagonist is an anti-human-IL-13 antibody or a human-IL-13-binding fragment thereof.

12. The method of claim 1, wherein the IL-13 antagonist is an anti-human-IL-13 antibody or a human-IL-13-binding fragment thereof, selected from:
(a) tralokinumab (BAK502G9, CAT-354) antibody;
(b) a human-IL-13-binding fragment of tralokinumab comprising an antibody antigen-binding site which is composed of a human antibody VH domain (SEQ ID NO: 15) and a human antibody VL domain (SEQ ID NO: 16) and which comprises a set of CDR's HCDR1, HCDR2, HCDR3, LCDR1, LCDR2 and LCDR3, wherein the VH domain comprises HCDR 1, HCDR2 and HCDR3 and the VL domain comprises LCDR1, LCDR2 and LCDR3, wherein the HCDR1 has the amino acid sequence of SEQ ID NO: 7, the HCDR2 has the amino acid sequence of SEQ ID NO: 8, the HCDR3 has the amino acid sequence of SEQ ID NO: 9, the LCDR1 has the amino acid sequence of SEQ ID NO: 10, the LCDR2 has the amino acid sequence of SEQ ID NO: 11, and the LCDR3 has the amino acid sequence of SEQ ID NO: 12;
(c) a human-IL-13-binding fragment of tralokinumab comprising an antibody antigen-binding site which is composed of a human antibody VH domain and a human antibody VL domain which comprises a set of CDR's HCDR1, HCDR2, HCDR3, LCDR1, LCDR2 and LCDR3, wherein the VH domain comprises HCDR 1, HCDR2 and HCDR3 and the VL domain comprises LCDR1, LCDR2 and LCDR3, wherein the HCDR1 has the amino acid sequence of SEQ ID NO: 7, the HCDR2 has the amino acid sequence of SEQ ID NO: 8, the HCDR3 has the amino acid sequence of SEQ ID NO: 9, the LCDR1 has the amino acid sequence of SEQ ID NO: 10, the LCDR2 has the amino acid sequence of SEQ ID NO: 11, and the LCDR3 has the amino acid sequence of SEQ ID NO: 12;
(d) an anti-human-IL-13 antibody, or a human-IL-13-binding fragment thereof comprising an antibody antigen-binding site which is composed of a human antibody VH domain (SEQ ID NO: 15) and/or a human antibody VL domain (SEQ ID NO: 16);
(e) an anti-human-IL-13 antibody or a human-IL-13 binding fragment thereof that competes with tralokinumab for binding to human IL-13;
(f) an anti-human-IL-13 antibody or a human-IL-13-binding fragment thereof capable of binding an epitope within the human IL-13 amino acid sequence from aspartic acid at position 99 to C-terminal asparagine at position 132 (FSSLHVRDTKIEVAQFVKDLLLHLKKLFREGRFN) of human IL-13 protein, or capable of binding an epitope within the human IL-13 sequence from aspartic acid at position 106 to C-terminal asparagine at position 132 (DTKIEVAQFVKDLLLHLKKLFREGRFN) of human IL-13 protein;
(g) an anti-human-IL-13 antibody or a human-IL-13-binding fragment thereof that has one, two, three, four, five, or six CDRs (SEQ ID NOs: 7, 8, 9, 10, 11, 12) in common with tralokinumab;
(h) an anti-human-IL-13 antibody or a human-IL-13-binding fragment thereof according to (a) to (g), wherein the whole antibody is IgG4;
(i) an anti-human-IL-13-antibody or a human-IL-13-binding fragment thereof, comprising one or more CDRs, or a set of all 6 CDRs selected from: BAK278D6 (SEQ ID NOS: 1, 2, 3, 4, 5, 6); (BAK1183H4 (SEQ ID NOS: 97, 98, 99, 100, 101, 102); BAK1167F02 (SEQ ID NOS: 64, 65, 66, 67, 68, 69); BAK1111D10 (SEQ ID NOS: 91, 92, 93, 94, 95, 96); BAK1166G02 (SEQ ID NOS: 67, 68, 69, 70, 71, 72); BAK1167F04 (SEQ ID NOS: 85, 86, 87, 88, 89, 90); BAK1184C8 (SEQ ID NOS: 73, 74, 75, 76, 77, 78); BAK1185E1 (SEQ ID NOS: 79, 80, 81, 82, 83, 84); and BAK1185F8 (SEQ ID NOS: 103, 104, 105, 106, 107, 108);
(j) an anti-human-IL-13 antibody or a human-IL-13-binding fragment thereof, comprising a VH domain comprising a set of CDRs and/or a VL domain comprising a set of CDRs selected from the set of HCDRs and/or set of LCDRs of BAK278D6, BAK1183H4, BAK1167F02, BAK1111D10, BAK1166G02, BAK1167F04, BAK1184C8, BAK1185E1 and BAK1185F8;
(k) an anti-human-IL-13 antibody or a human-IL-13 binding fragment thereof, comprising a VH and VL of a clone selected from: BAK278D6 (VH SEQ ID NO: 13, VL SEQ ID NO: 14), BAK1183H4 (VH SEQ ID NO: 37, VL SEQ ID NO: 38), BAK1167F02 (VH SEQ ID NO: 35, VL SEQ ID NO: 36), BAK1111D 10 (VH SEQ ID NO: 41, VL SEQ ID NO: 42), BAK1166G02 (VH SEQ ID NO: 53, VL SEQ ID NO: 54), BAK1167F04 (VH SEQ ID NO: 43, VL SEQ ID NO: 44), BAK1184C8 (VH SEQ ID NO: 45, VL SEQ ID NO: 46), BAK1185E1 (VH SEQ ID NO: 47, VL SEQ ID NO: 48), and BAK1185F8 (VH SEQ ID NO: 49, VL SEQ ID NO: 50);
(l) an anti-human-IL-13 antibody or a human-IL-13-binding fragment thereof according to (a) to (k), wherein HCDR1, HCDR2 and HCDR3 of the VH domain are within a germ-line framework and/or LCDR1, LCDR2 and LCDR3 of the VL domain are within a germ-line framework;
(m) an anti-human-IL-13 antibody or a human-IL-13-binding fragment thereof according to (a) to (l) wherein the HCDR1, HCDR2 and HCDR3 of the VH domain are within germ-line framework VH1 DP14; and
(n) an anti-human-IL-13 antibody or a human-IL-13-binding fragment thereof according to (a) to (m), wherein the LCDR1, LCDR2 and LCDR3 of the VL domain are within germ-line framework VL γ3 3H.

13-21. (canceled)

22. The method according to claim 12, wherein said antibody, or human-IL-13 binding fragment thereof, is selected from the group consisting of; an immunoglobulin molecule, a monoclonal antibody, a chimeric antibody, a CDR-grafted antibody, a humanized antibody, a Fab, a Fab', a F(ab′)2, a Fv, a disulfide linked Fv, a scFv, a single domain antibody, a diabody, a multispecific antibody, a dual-specific antibody, and a bispecific antibody.

23. A method according to claim 1, wherein the subject has impaired pulmonary function associated with asthma, COPD, or idiopathic pulmonary fibrosis.

24. A method according to claim 23, wherein the subject has impaired pulmonary function associated with asthma selected from atopic asthma non-atopic asthma, moderate to severe asthma, moderate to severe asthma uncontrolled by inhaled or oral corticosteroids and asthma at GINA score 5, 4, 3, 2, or 1.

25. A method according to claim 24, wherein the subject has impaired pulmonary function associated with asthma and is undergoing concomitant therapy with one or more further therapeutic selected from an inhaled corticosteroid, long-acting beta2 antagonist (LABA), theophylline, a leukotriene antagonist, an oral corticosteroid.

26-30. (canceled)

31. The method according to claim 1, wherein the IL-13 antagonist is an antibody or IL-13 binding fragment thereof administered at a dose in the range of from 100 to 800 mg subcutaneously.

32. (canceled)

33. The method according to claim 1, wherein the dose is selected from 150, 300, and 600 mg administered subcutaneously.

34. The method according to claim 1, wherein the IL-13 antagonist is an antibody or IL-13 binding fragment thereof administered intravenously at a dose in the range of from 100 to 1800 mg, or 200 to 1600 mg, e.g., 1500 mg.

35. The method according to claim 1, wherein the IL-13 antagonist is an antibody or IL-13 binding fragment thereof, administered at a frequency selected from every 1, 2, 3, 4, 6, 8, 10 and 12 weeks.

36-37. (canceled)

38. The method according to claim 1, wherein the IL-13 antagonist is an antibody or IL-13 binding fragment thereof, wherein an improvement in pulmonary function in asthma subjects can be detected by 15 days following initiation of treatment.

39. The method according to claim 1, wherein an improvement in pulmonary function in asthma subjects can be detected at least 12 weeks after cessation of treatment.

40. The method according to claim 1, wherein the IL-13 antagonist is an anti-human-IL-13 antibody or human-IL-13-binding fragment thereof and wherein the asthma is moderate to severe asthma (GINA 4 or 5).

41-50. (canceled)

51. The method according to claim 1, wherein the IL-13 antagonist is tralukinumab or a variant thereof or a human-IL-13 binding fragment thereof, administered by subcutaneous administration at a dose selected from 150 mg, 300 mg and 600 mg or by intravenous administration at a dose of 1500 mg at a dosing interval of 2 weeks or 4 weeks.

52-95. (canceled)

96. A method according to claim 1, wherein the IL-13 antagonist is selected from:
(a) an anti-human-IL-13 antibody selected from Lebrikizumab (MILR1444A/RG3637, Roche/Genentech), ABT-308 (Abbott), GSK679586 (GlaxoSmithkline) and QAX576 (Novartis);
(b) an anti-human-IL-13Ral antibody;
(c) an anti-human-IL-13Ral antibody Merck MK6105;
(d) an IL-13-toxin conjugate;
(e) IL-13-toxin conjugate IL-13 PE38QQR
(f) an IL-4 mutein;
(g) IL-4 mutein Aerovant™ (Aerovance) (modified IL-4);
(h) an anti-IL-4Ra antibody;
(i) anti-IL-4Ra antibody Regeneron-668 (Regeneron);
(j) a double-stranded oligonucleotide directed against IL-4Ra;
(k) AIR645 double-stranded oligonucleotide directed against IL-4Ra;
(l) an IL-4/IL-13 bispecific antibody;
(m) IL-4/IL-13 bispecific antibody 09/10 (Domantis); and,
(n) IL-4/IL-13 bispecific antibody DOM-1000P (Domantis).

97. A method for detecting a subject's response to an IL-13 antagonist treatment in asthma, comprising:
(a) assessing pulmonary function by a spirometric method prior to treatment,
(b) assessing pulmonary function by a spirometric method after treatment,
(c) wherein an improvement in pulmonary function is indicative of a beneficial response to treatment.

98. A method according to claim 97, wherein pulmonary function is assessed by measuring FEV1 or PEF.


SEQUENCE LISTING (249 sequences)

SEQ ID NO: 1 - protein, 5 aa, Homo sapiens
NYGLS

SEQ ID NO: 2 - protein, 17 aa, Homo sapiens
WISANNGDTNYGQEFQG

SEQ ID NO: 3 - protein, 13 aa, Homo sapiens
DSSSNWARWFFDL

SEQ ID NO: 4 - protein, 11 aa, Homo sapiens
GGNNIGSKLVH

SEQ ID NO: 5 - protein, 7 aa, Homo sapiens
DDGDRPS

SEQ ID NO: 6 - protein, 11 aa, Homo sapiens
QVWDTGSDPVV

SEQ ID NO: 7 - protein, 5 aa, Homo sapiens
NYGLS

SEQ ID NO: 8 - protein, 17 aa, Homo sapiens
WISANNGDTNYGQEFQG

SEQ ID NO: 9 - protein, 13 aa, Homo sapiens
DSSSSWARWFFDL

SEQ ID NO: 10 - protein, 11 aa, Homo sapiens
GGNIIGSKLVH

SEQ ID NO: 11 - protein, 7 aa, Homo sapiens
DDGDRPS

SEQ ID NO: 12 - protein, 11 aa, Homo sapiens
QVWDTGSDPVV

SEQ ID NO: 13 - protein, 122 aa, Homo sapiens
EVQLVQSGAEVKKPGASVKVSCKASGYTFRNYGLSWVRQAPGQGLEWMGWISANNGDTNYGQEFQGRITMTTETSTNTAHMELRSLRSDDTAVYYCVRDSSSNWARWFFDLWGKGTMVTVSS

SEQ ID NO: 14 - protein, 108 aa, Homo sapiens
SYVLTQPPSVSVAPGQTARIPCGGNNIGSKLVHWYQQKPGQAPVLVVYDDGDRPSGIPERFSGSNSGNTATLTISRIDAGDEADYYCQVWDTGSDPVVFGGGTKLTVL

SEQ ID NO: 15 - protein, 122 aa, Homo sapiens
QVQLVQSGAEVKKPGASVKVSCKASGYTFTNYGLSWVRQAPGQGLEWMGWISANNGDTNYGQEFQGRVTMTTDTSTSTAYMELRSLRSDDTAVYYCARDSSSSWARWFFDLWGRGTLVTVSS

SEQ ID NO: 16 - protein, 108 aa, Homo sapiens
SYVLTQPPSVSVAPGKTARITCGGNIIGSKLVHWYQQKPGQAPVLVIYDDGDRPSGIPERFSGSNSGNTATLTISRVEAGDEADYYCQVWDTGSDPVVFGGGTKLTVL

SEQ ID NO: 17 - protein, 30 aa, Homo sapiens
EVQLVQSGAEVKKPGASVKVSCKASGYTFR

SEQ ID NO: 18 - protein, 14 aa, Homo sapiens
WVRQAPGQGLEWMG

SEQ ID NO: 19 - protein, 32 aa, Homo sapiens
RITMTTETSTNTAHMELRSLRSDDTAVYYCVR

SEQ ID NO: 20 - protein, 22 aa, Homo sapiens
SYVLTQPPSVSVAPGQTARIPC

SEQ ID NO: 21 - protein, 15 aa, Homo sapiens
WYQQKPGQAPVLVVY

SEQ ID NO: 22 - protein, 32 aa, Homo sapiens
GIPERFSGSNSGNTATLTISRIDAGDEADYYC

SEQ ID NO: 23 - protein, 120 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARVGAAGEGYYGYWGRGTLVTVSS

SEQ ID NO: 24 - protein, 110 aa, Homo sapiens
NFMLTQPHSVSESPGKTVTISCTRSSGSIASNYVQWYQQRPGSAPTTVIYDDNQRPSGVPDRFSGSIDSSSNSASLTISGLKTEDEADYYCQSYDSNNDVFGGGTKVTVL

SEQ ID NO: 25 - protein, 123 aa, Homo sapiens
QVQLQESGGGGLVQPGGSLRLSCAASGFTFSSYGMSWVRQAPGKGLEWVSSISASGDSTFYADSVKGRFTISRDNNKNMVFLQVNSLRADDTAVYFCAKDWSQWLVGDAFDVWGRGTTVTVSS

SEQ ID NO: 26 - protein, 108 aa, Homo sapiens
DIQLTQSPSTLSASVGDRVTITCRASQSVSLWVAWYQQRPGKAPKLLIYDGSTLQSGVPARFSGSGSGTEFTLTISSLQPDDFATYYCQQYKTFSTFGQGTKVEIKRA

SEQ ID NO: 27 - protein, 30 aa, Homo sapiens
QVQLVQSGAEVKKPGASVKVSCKASGYTFT

SEQ ID NO: 28 - protein, 14 aa, Homo sapiens
WYRQAPGQGLEWMG

SEQ ID NO: 29 - protein, 32 aa, Homo sapiens
RVTMTTDTSTSTAYMELRSLRSDDTAVYYCAR

SEQ ID NO: 30 - protein, 22 aa, Homo sapiens
SYVLTQPPSVSVAPGKTARITC

SEQ ID NO: 31 - protein, 15 aa, Homo sapiens
WYQQKPGQAPVLVIY

SEQ ID NO: 32 - protein, 32 aa, Homo sapiens
GIPERFSGSNSGNTATLTISRVEAGDEADYYC

SEQ ID NO: 33 - protein, 122 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARVGKATTEEGYYGYWGRGTLVTVSS

SEQ ID NO: 34 - protein, 110 aa, Homo sapiens; identical to SEQ ID NO: 24

SEQ ID NO: 35 - protein, 122 aa, Homo sapiens
QVQLVQSGAEVKKPGASVKVSCKASGYTFEQTGVSWVRQAPGQGLEWMGWISANNGDTNYGQEFQGRVTMTTDTSTSTAYMELRSLRSDDTAVYYCARDSSSSWARWFFDLWGRGTLVTVSS

SEQ ID NO: 36 - protein, 108 aa, Homo sapiens; identical to SEQ ID NO: 16

SEQ ID NO: 37 - protein, 122 aa, Homo sapiens
QVQLVQSGAEVKKPGASVKVSCKASGYTFTNYGLSWVRQAPGQGLEWMGWINYDGGNTQYGQEFQGRVTMTTDTSTSTAYMELRSLRSDDTAVYYCARDSSSSWARWFFDLWGRGTLVTVSS

SEQ ID NO: 38 - protein, 108 aa, Homo sapiens; identical to SEQ ID NO: 16

SEQ ID NO: 39 - protein, 122 aa, Homo sapiens
QVQLVQSGAEVKKPGASVKVSCKASGYTFTNYGLSWVRQAPGQGLEWMGWISGLNGETLYGQEFQGRVTMTTDTSTSTAYMELRSLRSDDTAVYYCARDSSSSWARWFFDLWGRGTLVTVSS

SEQ ID NO: 40 - protein, 108 aa, Homo sapiens; identical to SEQ ID NO: 16

SEQ ID NO: 41 - protein, 122 aa, Homo sapiens
QVQLVQSGAEVKKPGASVKVSCKASGYTFTNYGLSWVRQAPGQGLEWMGWIATPDGQTSYGQEFQGRVTMTTDTSTSTAYMELRSLRSDDTAVYYCARDSNSSWARWFFDLWGRGTLVTVSS

SEQ ID NO: 42 - protein, 108 aa, Homo sapiens; identical to SEQ ID NO: 16

SEQ ID NO: 43 - protein, 122 aa, Homo sapiens
QVQLVQSGAEVKKPGASVKVSCKASGYTFIDTGVSWVRQAPGQGLEWMGWISANNGDTNYGQEFQGRVTMTTDTSTSTAYMELRSLRSDDTAVYYCARDSSSSWARWFFDLWGRGTLVTVSS

SEQ ID NO: 44 - protein, 108 aa, Homo sapiens; identical to SEQ ID NO: 16

SEQ ID NO: 45 - protein, 122 aa, Homo sapiens
QVQLVQSGAEVKKPGASVKVSCKASGYTFTNYGLSWVRQAPGQGLEWMGWISGSNGYTSYGQEFQGRVTMTTDTSTSTAYMELRSLRSDDTAVYYCARDSSSSWARWFFDLWGRGTLVTVSS

SEQ ID NO: 46 - protein, 108 aa, Homo sapiens; identical to SEQ ID NO: 16

SEQ ID NO: 47 - protein, 122 aa, Homo sapiens
QVQLVQSGAEVKKPGASVKVSCKASGYTFTNYGLSWVRQAPGQGLEWMGWINDATGDTQYGQEFQGRVTMTTDTSTSTAYMELRSLRSDDTAVYYCARDSSSSWARWFFDLWGRGTLVTVSS

SEQ ID NO: 48 - protein, 108 aa, Homo sapiens; identical to SEQ ID NO: 16

SEQ ID NO: 49 - protein, 122 aa, Homo sapiens
QVQLVQSGAEVKKPGASVKVSCKASGYTFTDYGLSWVRQAPGQGLEWMGWIRNIDGYTIYGQEFQGRVTMTTDTSTSTAYMELRSLRSDDTAVYYCARDSSSSWARWFFDLWGRGTLVTVSS

SEQ ID NO: 50 - protein, 108 aa, Homo sapiens; identical to SEQ ID NO: 16

SEQ ID NO: 51 - protein, 122 aa, Homo sapiens
QVQLVQSGAEVKKPGASVKVSCKASGYTFTNYGLSWVRQAPGQGLEWMGWIDDDSGTTIYGQEFQGRVTMTTDTSTSTAYMELRSLRSDDTAVYYCARDSSSSWARWFFDLWGRGTLVTVSS

SEQ ID NO: 52 - protein, 108 aa, Homo sapiens; identical to SEQ ID NO: 16

SEQ ID NO: 53 - protein, 122 aa, Homo sapiens
QVQLVQSGAEVKKPGASVKVSCKASGYTFANTGISWVRQAPGQGLEWMGWISANNGDTNYGQEFQGRVTMTTDTSTSTAYMELRSLRSDDTAVYYCARDSSSSWARWFFDLWGRGTLVTVSS

SEQ ID NO: 54 - protein, 108 aa, Homo sapiens; identical to SEQ ID NO: 16

SEQ ID NO: 55 - protein, 5 aa, Homo sapiens
SYAMS

SEQ ID NO: 56 - protein, 17 aa, Homo sapiens
AISGSGGSTYYADSVKG

SEQ ID NO: 57 - protein, 11 aa, Homo sapiens
VGAAGEGYYGY

SEQ ID NO: 58 - protein, 13 aa, Homo sapiens
TRSSGSIASNYVQ

SEQ ID NO: 59 - protein, 7 aa, Homo sapiens
DDNQRPS

SEQ ID NO: 60 - protein, 9 aa, Homo sapiens
QSYDSNNDV

SEQ ID NO: 61 - protein, 5 aa, Homo sapiens
QTGVS

SEQ ID NO: 62 - protein, 17 aa, Homo sapiens
WISANNGDTNYGQEFQG

SEQ ID NO: 63 - protein, 13 aa, Homo sapiens
DSSSSWARWFFDL

SEQ ID NO: 64 - protein, 11 aa, Homo sapiens
GGNIIGSKLVH

SEQ ID NO: 65 - protein, 7 aa, Homo sapiens
DDGDRPS

SEQ ID NO: 66 - protein, 11 aa, Homo sapiens
QVWDTGSDPVV

SEQ ID NO: 67 - protein, 5 aa, Homo sapiens
NTGIS

SEQ ID NO: 68 - protein, 17 aa, Homo sapiens
WISANNGDTNYGQEFQG

SEQ ID NO: 69 - protein, 13 aa, Homo sapiens
DSSSSWARWFFDL

SEQ ID NO: 70 - protein, 11 aa, Homo sapiens
GGNIIGSKLVH

SEQ ID NO: 71 - protein, 7 aa, Homo sapiens
DDGDRPS

SEQ ID NO: 72 - protein, 11 aa, Homo sapiens
QVWDTGSDPVV

SEQ ID NO: 73 - protein, 5 aa, Homo sapiens
NYGLS

SEQ ID NO: 74 - protein, 17 aa, Homo sapiens
WISGSNGYTSYGQEFQG

SEQ ID NO: 75 - protein, 13 aa, Homo sapiens
DSSSSWARWFFDL

SEQ ID NO: 76 - protein, 11 aa, Homo sapiens
GGNIIGSKLVH

SEQ ID NO: 77 - protein, 7 aa, Homo sapiens
DDGDRPS

SEQ ID NO: 78 - protein, 11 aa, Homo sapiens
QVWDTGSDPVV

SEQ ID NO: 79 - protein, 5 aa, Homo sapiens
NYGLS

SEQ ID NO: 80 - protein, 17 aa, Homo sapiens
WINDATGDTQYGQEFQG

SEQ ID NO: 81 - protein, 13 aa, Homo sapiens
DSSSSWARWFFDL

SEQ ID NO: 82 - protein, 11 aa, Homo sapiens
GGNIIGSKLVH

SEQ ID NO: 83 - protein, 7 aa, Homo sapiens
DDGDRPS

SEQ ID NO: 84 - protein, 11 aa, Homo sapiens
QVWDTGSDPVV

SEQ ID NO: 85 - protein, 5 aa, Homo sapiens
DTGVS

SEQ ID NO: 86 - protein, 17 aa, Homo sapiens
WISANNGDTNYGQEFQG

SEQ ID NO: 87 - protein, 13 aa, Homo sapiens
DSSSSWARWFFDL

SEQ ID NO: 88 - protein, 11 aa, Homo sapiens
GGNIIGSKLVH

SEQ ID NO: 89 - protein, 7 aa, Homo sapiens
DDGDRPS

SEQ ID NO: 90 - protein, 11 aa, Homo sapiens
QVWDTGSDPVV

SEQ ID NO: 91 - protein, 5 aa, Homo sapiens
NYGLS

SEQ ID NO: 92 - protein, 17 aa, Homo sapiens
WIATPDGQTSYGQEFQG

SEQ ID NO: 93 - protein, 13 aa, Homo sapiens
DSNSSWARWFFDL

SEQ ID NO: 94 - protein, 11 aa, Homo sapiens
GGNIIGSKLVH

SEQ ID NO: 95 - protein, 7 aa, Homo sapiens
DDGDRPS

SEQ ID NO: 96 - protein, 11 aa, Homo sapiens
QVWDTGSDPVV

SEQ ID NO: 97 - protein, 5 aa, Homo sapiens
NYGLS

SEQ ID NO: 98 - protein, 17 aa, Homo sapiens
WINYDGGNTQYGQEFQG

SEQ ID NO: 99 - protein, 13 aa, Homo sapiens
DSSSSWARWFFDL

SEQ ID NO: 100 - protein, 11 aa, Homo sapiens
GGNIIGSKLVH

SEQ ID NO: 101 - protein, 7 aa, Homo sapiens
DDGDRPS

SEQ ID NO: 102 - protein, 11 aa, Homo sapiens
QVWDTGSDPVV

SEQ ID NO: 103 - protein, 5 aa, Homo sapiens
DYGLS

SEQ ID NO: 104 - protein, 16 aa, Homo sapiens
WRINDGYTIYGQEFQG

SEQ ID NO: 105 - protein, 13 aa, Homo sapiens
DSSSSWARWFFDL

SEQ ID NO: 106 - protein, 11 aa, Homo sapiens
GGNIIGSKLVH

SEQ ID NO: 107 - protein, 7 aa, Homo sapiens
DDGDRPS

SEQ ID NO: 108 - protein, 11 aa, Homo sapiens
QVWDTGSDPVV

SEQ ID NO: 109 - DNA, 366 bp, Homo sapiens
caggtgcagctggtgcagtctggggctgaggtgaagaagcctggggcctcagtgaaggtctcctgcaaggcttctggttacacctttacaaattatggtctcagctgggtgcgacaggcccctggacaagggcttgagtggatgggatggatcagcgctaataatggcgacacaaattatggacaggaattccagggcagagtcaccatgaccacagatacatccacgagcacagcctacatggagttgaggagcctgagatctgacgacacggccgtttattactgtgcgagagactccagcagcaactgggcccgctggtttttcgatctctggggccgggggacactggtcaccgtctcctca

SEQ ID NO: 110 - DNA, 327 bp, Homo sapiens
tcctatgtgctgactcagccaccctcggtgtcagtggccccaggtaagacggccaggattacctgtgggggaaacaacattggaagtaaacttgtacactggtaccagcagaagccaggccaggcccctgtgctggtcatctatgatgatggcgaccggccctcagggatccctgagcgattctctggctccaactctgggaacacggccaccctgaccatcagcagggtcgaggccggggatgaggccgactattattgtcaggtgtgggatactggtagtgatcccgtggtattcggcggagggaccaagctgaccgtcctaggt

SEQ ID NO: 111 - DNA, 366 bp, Homo sapiens
caggtccagctggtgcagtctggggctgaggtgaagaagcctggggcctcagtgaaggtctcctgcaaggcttctggttacacctttacaaattatggtctcagctgggtgcgacaggcccctggacaagggcttgagtggatgggatggatcagcgctaataatggcgacacaaattatggacaggaattccagggcagagtcaccatgaccacagatacatccacgagcacagcctacatggagttgaggagcctgagatctgacgacacggccgtttattactgtgcgagagactccagcagcagctgggcccgctggtttttcgatctctggggccgggggacactggtcaccgtctcctca

SEQ ID NO: 112 - DNA, 327 bp, Homo sapiens
tcctatgtgctgactcagccaccctcggtgtcagtggccccaggaaagacggccaggattacctgtgggggaaacatcattggaagtaaacttgtacactggtaccagcagaagccaggccaggcccctgtgctggtcatctatgatgatggcgaccggccctcagggatccctgagcgattctctggctccaactctgggaacacggccaccctgaccatcagcagggtcgaggccggggatgaggccgactattattgtcaggtgtgggatactggtagtgatcccgtggtattcggcggagggaccaagctgaccgtcctaggt

SEQ ID NO: 113 - DNA, 366 bp, Homo sapiens
caggtccagctggtgcagtctggggctgaggtgaagaagcctggggcctcagtgaaggtctcctgcaaggcttctggttacacctttacaaattatggtctcagctgggtgcgacaggcccctggacaagggcttgagtggatgggatggatctccggcttgaacggcgagacattgtatggacaggaattccagggcagagtcaccatgaccacagatacatccacgagcacagcctacatggagttgaggagcctgagatctgacgacacggccgtttattactgtgcgagagactccagcagcagctgggcccgctggtttttcgatctctggggccgggggacactggtcaccgtctcctca

SEQ ID NO: 114 - DNA, 327 bp, Homo sapiens; identical to SEQ ID NO: 112

SEQ ID NO: 115 - DNA, 366 bp, Homo sapiens
caggtgcagctggtgcagtctggggctgaggtgaagaagcctggggcctcagtgaaggtctcctgcaaggcttctggttacacctttacaaattatggtctcagctgggtgcgacaggcccctggacaagggcttgagtggatgggatggatcgcaaccccagacggccagacaagctatggacaggaattccagggcagagtcaccatgaccacagatacatccacgagcacagcctacatggagttgaggagcctgagatctgacgacacggccgtttattactgtgcgagagactccaacagcagctgggcccgctggtttttcgatctctggggccgggggacactggtcaccgtctcctca

SEQ ID NO: 116 - DNA, 327 bp, Homo sapiens; identical to SEQ ID NO: 112

SEQ ID NO: 117 - DNA, 366 bp, Homo sapiens
caagtgcagctggtgcagtctggggctgaggtgaagaagcctggggcctcagtgaaggtctcctgcaaggcttctggttacacctttgagcagaccggcgtctcctgggtgcgacaggcccctggacaagggcttgagtggatgggatggatcagcgctaataatggcgacacaaattatggacaggaattccagggcagagtcaccatgaccacagatacatccacgagcacagcctacatggagttgaggagcctgagatctgacgacacggccgtttattactgtgcgagagactccagcagcagctgggcccgctggtttttcgatctctggggccgggggacactggtcaccgtctcctca

SEQ ID NO: 118 - DNA, 327 bp, Homo sapiens; identical to SEQ ID NO: 112

SEQ ID NO: 119 - DNA, 366 bp, Homo sapiens
caggtgcagctggtgcagtctggggctgaggtgaagaagcctggggcctcagtgaaggtctcctgcaaggcttctggttacacctttatcgacaccggggtctcctgggtgcgacaggcccctggacaagggcttgagtggatgggatggatcagcgctaataatggcgacacaaattatggacaggaattccagggcagagtcaccatgaccacagatacatccacgagcacagcctacatggagttgaggagcctgagatctgacgacacggccgtttattactgtgcgagagactccagcagcagctgggcccgctggtttttcgatctctggggccgggggacactggtcaccgtctcctca

SEQ ID NO: 120 - DNA, 327 bp, Homo sapiens; identical to SEQ ID NO: 112

SEQ ID NO: 121 - DNA, 366 bp, Homo sapiens
caggtgcagctggtgcagtctggggctgaggtgaagaagcctggggcctcagtgaaggtctcctgcaaggcttctggttacacctttacaaattatggtctcagctgggtgcgacaggcccctggacaagggcttgagtggatgggatggatcaactacgacggcggcaacacacagtatggacaggaattccagggcagagtcaccatgaccacagatacatccacgagcacagcctacatggagttgaggagcctgagatctgacgacacggccgtttattactgtgcgagagactccagcagcagctgggcccgctggtttttcgatctctggggccgggggacactggtcaccgtctcctca

SEQ ID NO: 122 - DNA, 327 bp, Homo sapiens; identical to SEQ ID NO: 112

SEQ ID NO: 123 - DNA, 366 bp, Homo sapiens
caggtgcagctggtgcagtctggggctgaggtgaagaagcctggggcctcagtgaaggtctcctgcaaggcttctggttacacctttacaaattatggtctcagctgggtgcgacaggcccctggacaagggcttgagtggatgggatggatcagcgggagcaacggctacacatcttatggacaggaattccagggcagagtcaccatgaccacagatacgtccacgagcacagcctacatggagttgaggagcctgagatctgacgacacggccgtttattactgtgcgagagactccagcagcagctgggcccgctggtttttcgatctctggggccgggggacactggtcaccgtctcctca

SEQ ID NO: 124 - DNA, 327 bp, Homo sapiens; identical to SEQ ID NO: 112

SEQ ID NO: 125 - DNA, 366 bp, Homo sapiens
caggtgcagctggtgcagtctggggctgaggtgaagaagcctggggcctcagtgaaggtctcctgcaaggcttctggttacacctttacaaattatggtctcagctgggtgcgacaggcccctggacaagggcttgagtggatgggatggatcaacgacgccaccggcgacacacagtatggacaggaattccagggcagagtcaccatgaccacagatacatccacgagcacagcctacatggagttgaggagcctgagatctgacgacacggccgtttattactgtgcgagagactccagcagcagctgggcccgctggtttttcgatctctggggccgggggacactggtcaccgtctcctca

SEQ ID NO: 126 - DNA, 327 bp, Homo sapiens; identical to SEQ ID NO: 112

SEQ ID NO: 127 - DNA, 366 bp, Homo sapiens
caggtgcagctggtgcagtctggggctgaggtgaagaagcctggggcctcagtgaaggtctcctgcaaggcttctggttacacctttacagattatggtctcagctgggtgcgacaggcccctggacaagggctagagtggatgggatggatccgcaacatcgacggctacacaatttatggacaggaattccagggcagagtcaccatgaccacagatacatccacgagcacagcctacatggagttgaggagcctgagatctgacgacacggccgtttattactgtgcgagagactccagcagcagctgggcccgctggtttttcgatctctggggccgggggacactggtcaccgtctcctca

SEQ ID NO: 128 - DNA, 327 bp, Homo sapiens; identical to SEQ ID NO: 112

SEQ ID NO: 129 - DNA, 366 bp, Homo sapiens
caggtccagctggtgcagtctggggctgaggtgaagaagcctggggcctcagtgaaggtctcctgcaaggcttctggttacacctttacaaattatggtctcagctgggtgcgacaggcccctggacaagggcttgagtggatgggatggatcgacgacgacagcggcacgacaatatatggacaggaattccagggcagagtcaccatgaccacagatacatccacgagcacagcctacatggagttgaggagcctgagatctgacgacacggccgtttattactgtgcgagagactccagcagcagctgggcccgctggtttttcgatctctggggccgggggacactggtcaccgtctcctca

SEQ ID NO: 130 - DNA, 327 bp, Homo sapiens; identical to SEQ ID NO: 112

SEQ ID NO: 131 - DNA, 366 bp, Homo sapiens
caagtgcagttggtgcagtctggggctgaggtgaagaagcctggggcctcagtgaaggtctcctgcaaggcttctggttacacctttgcgaacaccgggatctcgtgggtgcgacaggcccctggacaagggcttgagtggatgggatggatcagcgctaataatggcgacacaaattatggacaggaattccagggcagagtcaccatgaccacagatacatccacgagcacagcctacatggagttgaggagcctgagatctgacgacacggccgtttattactgtgcgagagactccagcagcagctgggcccgctggtttttcgatctctggggtcgggggacactggtcaccgtctcctca

SEQ ID NO: 132 - DNA, 327 bp, Homo sapiens; identical to SEQ ID NO: 112

SEQ ID NO: 133 - protein, 122 aa, Homo sapiens
EVQLVQSGAEVKKPGASVKVSCKASGYTFTNYGLSWVRQAPGQGLEWMGWISANNGETNYGQEFQGRVTMTTETPTNTAHMELRSLTSDDTAVYYCVRDSSSNWARWYFDLWGQGTLVTVSS

SEQ ID NO: 134 - protein, 109 aa, Homo sapiens
SYVLTQPPSVSVAPGQTARIPCGGNNIGSKLVHWYQQKPGQAPVLVVYDDGDRPSGIPERFSGSNSGNTATLTISRIDAGDEADYYCQVWDTGSDPVVFGGGTKLTVLG

SEQ ID NO: 135 - protein, 5 aa, Homo sapiens
NYGLS

SEQ ID NO: 136 - protein, 17 aa, Homo sapiens
WISANNGETNYGQEFQG

SEQ ID NO: 137 - protein, 13 aa, Homo sapiens
DSSSNWARWYFDL

SEQ ID NO: 138 - protein, 11 aa, Homo sapiens
GGNNIGSKLVH

SEQ ID NO: 139 - protein, 7 aa, Homo sapiens
DDGDRPS

SEQ ID NO: 140 - protein, 11 aa, Homo sapiens
QVWDTGSDPVV

SEQ ID NO: 141 - protein, 5 aa, Homo sapiens
SYAMS

SEQ ID NO: 142 - protein, 17 aa, Homo sapiens
AISGSGGSTYYADSVKG

SEQ ID NO: 143 - protein, 11 aa, Homo sapiens
VGAAGEGYYGY

SEQ ID NO: 144 - protein, 13 aa, Homo sapiens
TRSSGSIASNYVE

SEQ ID NO: 145 - protein, 7 aa, Homo sapiens
DDNQRPS

SEQ ID NO: 146 - protein, 9 aa, Homo sapiens
QSYDSNNDV

SEQ ID NO: 147 - protein, 5 aa, Homo sapiens
SYAMS

SEQ ID NO: 148 - protein, 17 aa, Homo sapiens
AISGSGGSTYYADSVKG

SEQ ID NO: 149 - protein, 13 aa, Homo sapiens
VGRATTDEGYYGY

SEQ ID NO: 150 - protein, 13 aa, Homo sapiens
TRSSGSIASNYVQ

SEQ ID NO: 151 - protein, 7 aa, Homo sapiens
DDNQRPS

SEQ ID NO: 152 - protein, 9 aa, Homo sapiens
QSYDSNNDV

SEQ ID NO: 153 - protein, 5 aa, Homo sapiens
SYAMS

SEQ ID NO: 154 - protein, 17 aa, Homo sapiens
AISGSGGSTYYADSVKG

SEQ ID NO: 155 - protein, 11 aa, Homo sapiens
VGKATTEEGYY

SEQ ID NO: 156 - protein, 13 aa, Homo sapiens
TRSSGSIASNYVQ

SEQ ID NO: 157 - protein, 7 aa, Homo sapiens
DDNQRPS

SEQ ID NO: 158 - protein, 9 aa, Homo sapiens
QSYDSNNDV

SEQ ID NO: 159 - DNA, 15 bp, Homo sapiens
aattatggtctcagc

SEQ ID NO: 160 - DNA, 51 bp, Homo sapiens
tggatcagcgctaataatggcgacacaaattatggacaggaattccagggc

SEQ ID NO: 161 - DNA, 39 bp, Homo sapiens
gactccagcagcaactgggcccgctggtttttcgatctc

SEQ ID NO: 162 - DNA, 33 bp, Homo sapiens
gggggaaacaacattggaagtaaacttgtacac

SEQ ID NO: 163 - DNA, 21 bp, Homo sapiens
gatgatggcgaccggccctca

SEQ ID NO: 164 - DNA, 33 bp, Homo sapiens
caggtgtgggatactggtagtgatcccgtggta

SEQ ID NO: 165 - DNA, 15 bp, Homo sapiens
aattatggtctcagc

SEQ ID NO: 166 - DNA, 51 bp, Homo sapiens; identical to SEQ ID NO: 160

SEQ ID NO: 167 - DNA, 39 bp, Homo sapiens
gactccagcagcagctgggcccgctggtttttcgatctc

SEQ ID NO: 168 - DNA, 33 bp, Homo sapiens
gggggaaacatcattggaagtaaacttgtacac

SEQ ID NO: 169 - DNA, 21 bp, Homo sapiens
gatgatggcgaccggccctca

SEQ ID NO: 170 - DNA, 33 bp, Homo sapiens; identical to SEQ ID NO: 164

SEQ ID NO: 171 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 172 - protein, 105 aa, Homo sapiens
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 173 - protein, 132 aa, Homo sapiens
MALLLTTVIALTCLGGFASPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 174 - protein, 132 aa, Macaca fascicularis
MALLLTTVIALTCLGGFASPSPVPPSTALKELIEELVNITQNQKAPLCNGSMVWSINLTAGVYCAALESLINVSGCSAIEKTQRMLNGFCPHKVSAGQFSSLRVRDTKIEVAQFVKDLLVHLKKLFREGQFN

SEQ ID NO: 175 - protein, 131 aa, Mus sp.
MALWVTAVLALACLGGLAAPGPVPRSVSLPLTLKELIEELSNITQDQTPLCNGSMVWSVDLAAGGFCVALDSLTNISNCNAIYRTQRILHGLCNRKAPTTVSSLPDTKIEVAHFITKLLSYTKQLFRHGPF

SEQ ID NO: 176 - protein, 5 aa, Artificial Sequence
  Description of Artificial Sequence Synthetic HCDR1 formula peptide
XXGXS

SEQ ID NO: 177 - protein, 17 aa, Artificial Sequence
  Description of Artificial Sequence Synthetic HCDR2 formula peptide
WIXXXXGXTXYXXXFXX

SEQ ID NO: 178 - protein, 13 aa, Artificial Sequence
  Description of Artificial Sequence Synthetic HCDR3 formula peptide
DXXXXWARWXFXL

SEQ ID NO: 179 - protein, 11 aa, Artificial Sequence
  Description of Artificial Sequence Synthetic LCDR1 formula peptide
GGXXXGXXLVH

SEQ ID NO: 180 - protein, 7 aa, Artificial Sequence
  Description of Artificial Sequence Synthetic LCDR2 formula peptide
DDGDRPX

SEQ ID NO: 181 - protein, 11 aa, Artificial Sequence
  Description of Artificial Sequence Synthetic LCDR3 formula peptide
QVWDTGSXPVX

SEQ ID NO: 182 - protein, 5 aa, Homo sapiens
LTGVS

SEQ ID NO: 183 - protein, 5 aa, Homo sapiens
GTGVS

SEQ ID NO: 184 - protein, 5 aa, Homo sapiens
ETGIS

SEQ ID NO: 185 - protein, 5 aa, Homo sapiens
DTGIS

SEQ ID NO: 186 - protein, 5 aa, Homo sapiens
GTGIS

SEQ ID NO: 187 - protein, 5 aa, Homo sapiens
NYGFS

SEQ ID NO: 188 - protein, 17 aa, Homo sapiens
WIRPTDGLTMYGQEFQG

SEQ ID NO: 189 - protein, 17 aa, Homo sapiens
WIDDRTGTTQYGQEFQG

SEQ ID NO: 190 - protein, 17 aa, Homo sapiens
WIRASDGQTIYGQEFQG

SEQ ID NO: 191 - protein, 17 aa, Homo sapiens
WISGIDGVTLYGQEFQG

SEQ ID NO: 192 - protein, 17 aa, Homo sapiens
WIRAADGETHYGQEFQG

SEQ ID NO: 193 - protein, 17 aa, Homo sapiens
WIGNNNGDTLYGQEFQG

SEQ ID NO: 194 - protein, 17 aa, Homo sapiens
WIGPSKGETSYGQEFQG

SEQ ID NO: 195 - protein, 17 aa, Homo sapiens
WIRPRDGTTHYGQEFQG

SEQ ID NO: 196 - protein, 17 aa, Homo sapiens
WISGRSGATLYGQEFQG

SEQ ID NO: 197 - protein, 17 aa, Homo sapiens
WIEGSTGNTIYGQEFQG

SEQ ID NO: 198 - protein, 17 aa, Homo sapiens
WIGPINGMTHYGQEFQG

SEQ ID NO: 199 - protein, 17 aa, Homo sapiens
WISANNGDTNYGQKFQG

SEQ ID NO: 200 - protein, 17 aa, Homo sapiens
WISANNGDTDYGQEFQG

SEQ ID NO: 201 - protein, 17 aa, Homo sapiens
WISANNGGTNYGQEFQG

SEQ ID NO: 202 - protein, 17 aa, Homo sapiens
WISANNGDTNYRQEFQG

SEQ ID NO: 203 - protein, 17 aa, Homo sapiens
WIGANNGDTNYGQEFQG

SEQ ID NO: 204 - protein, 17 aa, Homo sapiens
WISANNGDTNYGQEFRG

SEQ ID NO: 205 - protein, 17 aa, Homo sapiens
WISANNGDTIYGQEFQG

SEQ ID NO: 206 - protein, 17 aa, Homo sapiens
WISTNNGDTNYGQEFQG

SEQ ID NO: 207 - protein, 17 aa, Homo sapiens
WISANNGDTIYRQEFQG

SEQ ID NO: 208 - protein, 17 aa, Homo sapiens
WISANNGDTSYGQEFQG

SEQ ID NO: 209 - protein, 17 aa, Homo sapiens
WISANNGDTKYGQEFQG

SEQ ID NO: 210 - protein, 17 aa, Homo sapiens
WIGANNGDTDYGQEFQG

SEQ ID NO: 211 - protein, 17 aa, Homo sapiens
WISANIGDTNYGQEFQG

SEQ ID NO: 212 - protein, 17 aa, Homo sapiens
WISANNGDTNYGQGFQG

SEQ ID NO: 213 - protein, 17 aa, Homo sapiens
WISTNNGDTNYGREFQG

SEQ ID NO: 214 - protein, 17 aa, Homo sapiens
WISANNGDTNYGQEFQK

SEQ ID NO: 215 - protein, 13 aa, Homo sapiens
DSDSSWARWFFDL

SEQ ID NO: 216 - protein, 13 aa, Homo sapiens
DSTSAWARWFFDL

SEQ ID NO: 217 - protein, 13 aa, Homo sapiens
DSNSAWARWFFDL

SEQ ID NO: 218 - protein, 13 aa, Homo sapiens
DSSSIWARWFFDL

SEQ ID NO: 219 - protein, 13 aa, Homo sapiens
DSTSRWARWFFDL

SEQ ID NO: 220 - protein, 13 aa, Homo sapiens
DDPRPWARWFFDL

SEQ ID NO: 221 - protein, 13 aa, Homo sapiens
DSSSKWARWFFDL

SEQ ID NO: 222 - protein, 13 aa, Homo sapiens
DSNSNWARWFFYL

SEQ ID NO: 223 - protein, 13 aa, Homo sapiens
DSNSSWARWFFDL

SEQ ID NO: 224 - protein, 13 aa, Homo sapiens
DRDSSWARWFFDL

SEQ ID NO: 225 - protein, 11 aa, Homo sapiens
GGNLIGSKLVH

SEQ ID NO: 226 - protein, 11 aa, Homo sapiens
GGNMIGSKLVH

SEQ ID NO: 227 - protein, 11 aa, Homo sapiens
GGNCIGSKLVH

SEQ ID NO: 228 - protein, 11 aa, Homo sapiens
GGNVIGSKLVH

SEQ ID NO: 229 - protein, 11 aa, Homo sapiens
GGNKIGSKLVH

SEQ ID NO: 230 - protein, 11 aa, Homo sapiens
GGNYIGSKLVH

SEQ ID NO: 231 - protein, 11 aa, Homo sapiens
GGNFIGSKLVH

SEQ ID NO: 232 - protein, 11 aa, Homo sapiens
GGNRIGSKLVH

SEQ ID NO: 233 - protein, 11 aa, Homo sapiens
GGNTIGSKLVH

SEQ ID NO: 234 - protein, 11 aa, Homo sapiens
GGNNIGGKLVH

SEQ ID NO: 235 - protein, 11 aa, Homo sapiens
GGNSIGSRLVH

SEQ ID NO: 236 - protein, 11 aa, Homo sapiens
GGDNIGGKLVH

SEQ ID NO: 237 - protein, 11 aa, Homo sapiens
GGNSIGGKLVH

SEQ ID NO: 238 - protein, 11 aa, Homo sapiens
GGNAIGSKLVH

SEQ ID NO: 239 - protein, 11 aa, Homo sapiens
GGNSIGSKLVH

SEQ ID NO: 240 - protein, 11 aa, Homo sapiens
GGNHIGSKLVH

SEQ ID NO: 241 - protein, 11 aa, Homo sapiens
GGNGIGSKLVH

SEQ ID NO: 242 - protein, 11 aa, Homo sapiens
GGSNIGGKLVH

SEQ ID NO: 243 - protein, 11 aa, Homo sapiens
GGNNVGGKLVH

SEQ ID NO: 244 - protein, 11 aa, Homo sapiens
GGNNIGSRLVH

SEQ ID NO: 245 - protein, 7 aa, Homo sapiens
DDGDRPT

SEQ ID NO: 246 - protein, 11 aa, Homo sapiens
QVWDTGSNPVV

SEQ ID NO: 247 - protein, 11 aa, Homo sapiens
QVWDTGSDPVI

SEQ ID NO: 248 - protein, 132 aa, Artificial Sequence
  Description of Artificial Sequence Synthetic consensus polypeptide between human IL-3 and Cynomolgus IL-3
MALLLTTVIALTCLGGFASPXPVPPSTALXELIEELVNITQNQKAPLCNGSMVWSINLTAGXYCAALESLINVSGCSAIEKTQRMLXGFCPHKVSAGQFSSLXVRDTKIEVAQFVKDLLXHLKKLFREGXFN

SEQ ID NO: 249 - protein, 136 aa, Artificial Sequence
  Description of Artificial Sequence Synthetic consensus polypeptide between human IL-3 and murine IL-3
MALXXTXVXALXCLGGXAXPGPVPXSXXXXXXLXELIEELXNITQXQXXPLCNGSMVWSXXLXAGXXCXALXSLXNXSXCXAIXXTQRXLXGXCXXKXXXXXXSSLXXXDTKIEVAXFXXXLLXXXKXLFRXGXFX